(19) United States
(12) Patent Application Publication
(10) Pub. No.: US 2024/0117072 A1
(43) Pub. Date: Apr. 11, 2024

(54) FAP BINDING MOLECULES AND USES THEREOF

(71) Applicant: BioArdis, LLC, San Diego, CA (US)

(72) Inventors: Farzad Haerizadeh, San Diego, CA (US); Masood Tayebi, San Diego, CA (US)

(21) Appl. No.: 18/258,203

(22) PCT Filed: Dec. 16, 2021
(86) PCT No.: PCT/US2021/063821

§ 371 Date: Jun. 16, 2023

Related U.S. Application Data
(60) Provisional application No. 63/262,706, filed on Oct. 19, 2021.
(60) Provisional application No. 63/127,885, filed on Dec. 18, 2020.

(51) Int. Cl. C07K 16/40; A61K 39/00; C07K 14/705; C07K 14/725

(52) U.S. Cl. CPC C07K 16/40; A61K 39/4611; A61K 39/4622; A61K 39/4631; A61K 39/464411; A61K 39/46446; C07K 14/7051; C07K 14/70517; C07K 14/70521; C07K 14/70578; A61K 2239/17; A61K 2239/21; A61K 2239/29; C07K 2317/24; C07K 2317/53; C07K 2317/565; C07K 2317/569; C07K 2319/02; C07K 2319/03

(57) ABSTRACT

Disclosed herein are fibroblast activation protein (FAP)-specific binding polypeptides. These binding polypeptides may be incorporated into chimeric antigen receptors (CARs). Also disclosed herein are methods of using these binding polypeptides and/or CARs for the treatment of, for example, a cancer.

### CROSS-REFERENCE TO RELATED APPLICATIONS

This application claims the benefit of priority of U.S. Provisional Patent Application No. 63/127,885, filed Dec. 18, 2020, and U.S. Provisional Patent Application No. 63/262,706, filed Oct. 19, 2021, each of which is hereby expressly incorporated by reference in its entirety.

### REFERENCE TO SEQUENCE LISTING

The present application is being filed along with a Sequence Listing in electronic format. The Sequence Listing is provided as a file entitled BWGBO11_SeqListing.TXT, which was created and last modified on Dec. 16, 2021, which is 187,259 bytes in size. The information in the electronic Sequence Listing is hereby incorporated by reference in its entirety.

### FIELD

Aspects of the present disclosure relate generally to fibroblast activation protein (FAP)-specific binding polypeptides. These binding polypeptides can be incorporated into chimeric antigen receptor (CAR) constructs to be expressed in immune cells. These binding polypeptides and CARs may be used in the treatment of cancer.

### BACKGROUND

Chimeric antigen receptor (CAR) T cells and other adoptive cell therapies have been shown to be effective in the treatment of cancer. The CAR, which is made up of an extracellular antigen binding domain, a transmembrane domain, and an intracellular signaling domain, enables directed killing of cancer cells based on cell surface antigen expression while minimally affecting normal cells that are not expressing the targeted antigen. The extracellular antigen binding domain is often made up of an antibody or a binding fragment or derivative thereof, such as a single chain variable fragment (scFv) or single domain antibody (sdAb). There is a present need for improved extracellular antigen binding domains to be used in CARs for the treatment of various cancers or other diseases.

### SUMMARY OF THE DISCLOSURE

Disclosed herein are binding polypeptides that are able to bind to fibroblast activation protein (FAP). These binding polypeptides may be incorporated in a chimeric antigen receptor (CAR), which can be expressed by a cell. In some embodiments, the binding polypeptides are single domain antibodies (sdAbs).

Disclosed herein in some embodiments are FAP binding polypeptides comprising an immunoglobulin heavy chain variable domain comprising a CDR-H1, CDR-H2, and CDR-H3. In some embodiments, the CDR-H1 comprises a sequence having at least 90%, 95%, 99%, or 100% sequence identity to a sequence selected from SEQ ID NOs: 1-108. In some embodiments, the CDR-H2 comprises a sequence having at least 90%, 95%, 99%, or 100% sequence identity to a sequence selected from SEQ ID NOs: 109-216. In some embodiments, the CDR-H3 comprises a sequence having at least 90%, 95%, 99%, or 100% sequence identity to a sequence selected from SEQ ID NOs: 217-324. In some embodiments, the CDR-H1 comprises a sequence having at least 90%, 95%, 99%, or 100% sequence identity to a sequence selected from SEQ ID NOs: 1-108, the CDR-H2 comprises a sequence having at least 90%, 95%, 99%, or 100% sequence identity to a sequence selected from SEQ ID NOs: 109-216, and the CDR-H3 comprises a sequence having at least 90%, 95%, 99%, or 100% sequence identity to a sequence selected from SEQ ID NOs: 217-324. In some embodiments, the immunoglobulin heavy chain variable domain comprises a sequence having at least 90%, 95%, 99%, or 100% sequence identity to a sequence selected from SEQ ID NOs: 325-432.

Also disclosed herein are nucleic acids that encode for any one of the FAP binding polypeptides disclosed herein.

Also disclosed herein are methods of treating a cancer in a subject in need thereof. In some embodiments, the methods comprise administering a chimeric antigen receptor cell to the subject. In some embodiments, the chimeric antigen receptor cell is any one of the chimeric antigen receptor cells disclosed herein. In some embodiments, the chimeric antigen receptor cell comprises any one or more of the FAP binding polypeptides disclosed herein.

Embodiments of the present invention provided herein are described by way of the following numbered alternatives:

1. A fibroblast activation protein (FAP) binding polypeptide comprising an immunoglobulin heavy chain variable domain comprising a CDR-H1, CDR-H2, and CDR-H3, wherein:
the CDR-H1 comprises a sequence having at least 90%, 95%, 99%, or 100% sequence identity to a sequence selected from SEQ ID NOs: 1-108;
the CDR-H2 comprises a sequence having at least 90%, 95%, 99%, or 100% sequence identity to a sequence selected from SEQ ID NOs: 109-216; and
the CDR-H3 comprises a sequence having at least 90%, 95%, 99%, or 100% sequence identity to a sequence selected from SEQ ID NOs: 217-324.

2. The FAP binding polypeptide of alternative 1, wherein:
1) the CDR-H1 comprises the sequence of SEQ ID NO: 1, the CDR-H2 comprises the sequence of SEQ ID NO: 109, and the CDR-H3 comprises the sequence of SEQ ID NO: 217;
2) the CDR-H1 comprises the sequence of SEQ ID NO: 2, the CDR-H2 comprises the sequence of SEQ ID NO: 110, and the CDR-H3 comprises the sequence of SEQ ID NO: 218;
3) the CDR-H1 comprises the sequence of SEQ ID NO: 3, the CDR-H2 comprises the sequence of SEQ ID NO: 111, and the CDR-H3 comprises the sequence of SEQ ID NO: 219;
4) the CDR-H1 comprises the sequence of SEQ ID NO: 4, the CDR-H2 comprises the sequence of SEQ ID NO: 112, and the CDR-H3 comprises the sequence of SEQ ID NO: 220;
5) the CDR-H1 comprises the sequence of SEQ ID NO: 5, the CDR-H2 comprises the sequence of SEQ ID NO: 113, and the CDR-H3 comprises the sequence of SEQ ID NO: 221;
6) the CDR-H1 comprises the sequence of SEQ ID NO: 6, the CDR-H2 comprises the sequence of SEQ ID NO: 114, and the CDR-H3 comprises the sequence of SEQ ID NO: 222;
7) the CDR-H1 comprises the sequence of SEQ ID NO: 7, the CDR-H2 comprises the sequence of SEQ ID NO: 115, and the CDR-H3 comprises the sequence of SEQ ID NO: 223;
8) the CDR-H1 comprises the sequence of SEQ ID NO: 8, the CDR-H2 comprises the sequence of SEQ ID NO: 116, and the CDR-H3 comprises the sequence of SEQ ID NO: 224;
9) the CDR-H1 comprises the sequence of SEQ ID NO: 9, the CDR-H2 comprises the sequence of SEQ ID NO: 117, and the CDR-H3 comprises the sequence of SEQ ID NO: 225;
10) the CDR-H1 comprises the sequence of SEQ ID NO: 10, the CDR-H2 comprises the sequence of SEQ ID NO: 118, and the CDR-H3 comprises the sequence of SEQ ID NO: 226;
11) the CDR-H1 comprises the sequence of SEQ ID NO: 11, the CDR-H2 comprises the sequence of SEQ ID NO: 119, and the CDR-H3 comprises the sequence of SEQ ID NO: 227;
12) the CDR-H1 comprises the sequence of SEQ ID NO: 12, the CDR-H2 comprises the sequence of SEQ ID NO: 120, and the CDR-H3 comprises the sequence of SEQ ID NO: 228;
13) the CDR-H1 comprises the sequence of SEQ ID NO: 13, the CDR-H2 comprises the sequence of SEQ ID NO: 121, and the CDR-H3 comprises the sequence of SEQ ID NO: 229;
14) the CDR-H1 comprises the sequence of SEQ ID NO: 14, the CDR-H2 comprises the sequence of SEQ ID NO: 122, and the CDR-H3 comprises the sequence of SEQ ID NO: 230;
15) the CDR-H1 comprises the sequence of SEQ ID NO: 15, the CDR-H2 comprises the sequence of SEQ ID NO: 123, and the CDR-H3 comprises the sequence of SEQ ID NO: 231;
16) the CDR-H1 comprises the sequence of SEQ ID NO: 16, the CDR-H2 comprises the sequence of SEQ ID NO: 124, and the CDR-H3 comprises the sequence of SEQ ID NO: 232;
17) the CDR-H1 comprises the sequence of SEQ ID NO: 17, the CDR-H2 comprises the sequence of SEQ ID NO: 125, and the CDR-H3 comprises the sequence of SEQ ID NO: 233;
18) the CDR-H1 comprises the sequence of SEQ ID NO: 18, the CDR-H2 comprises the sequence of SEQ ID NO: 126, and the CDR-H3 comprises the sequence of SEQ ID NO: 234;
19) the CDR-H1 comprises the sequence of SEQ ID NO: 19, the CDR-H2 comprises the sequence of SEQ ID NO: 127, and the CDR-H3 comprises the sequence of SEQ ID NO: 235;
20) the CDR-H1 comprises the sequence of SEQ ID NO: 20, the CDR-H2 comprises the sequence of SEQ ID NO: 128, and the CDR-H3 comprises the sequence of SEQ ID NO: 236;
21) the CDR-H1 comprises the sequence of SEQ ID NO: 21, the CDR-H2 comprises the sequence of SEQ ID NO: 129, and the CDR-H3 comprises the sequence of SEQ ID NO: 237;
22) the CDR-H1 comprises the sequence of SEQ ID NO: 22, the CDR-H2 comprises the sequence of SEQ ID NO: 130, and the CDR-H3 comprises the sequence of SEQ ID NO: 238;
23) the CDR-H1 comprises the sequence of SEQ ID NO: 23, the CDR-H2 comprises the sequence of SEQ ID NO: 131, and the CDR-H3 comprises the sequence of SEQ ID NO: 239;
24) the CDR-H1 comprises the sequence of SEQ ID NO: 24, the CDR-H2 comprises the sequence of SEQ ID NO: 132, and the CDR-H3 comprises the sequence of SEQ ID NO: 240;
25) the CDR-H1 comprises the sequence of SEQ ID NO: 25, the CDR-H2 comprises the sequence of SEQ ID NO: 133, and the CDR-H3 comprises the sequence of SEQ ID NO: 241;
26) the CDR-H1 comprises the sequence of SEQ ID NO: 26, the CDR-H2 comprises the sequence of SEQ ID NO: 134, and the CDR-H3 comprises the sequence of SEQ ID NO: 242;
27) the CDR-H1 comprises the sequence of SEQ ID NO: 27, the CDR-H2 comprises the sequence of SEQ ID NO: 135, and the CDR-H3 comprises the sequence of SEQ ID NO: 243;
28) the CDR-H1 comprises the sequence of SEQ ID NO: 28, the CDR-H2 comprises the sequence of SEQ ID NO: 136, and the CDR-H3 comprises the sequence of SEQ ID NO: 244;
29) the CDR-H1 comprises the sequence of SEQ ID NO: 29, the CDR-H2 comprises the sequence of SEQ ID NO: 137, and the CDR-H3 comprises the sequence of SEQ ID NO: 245;
30) the CDR-H1 comprises the sequence of SEQ ID NO: 30, the CDR-H2 comprises the sequence of SEQ ID NO: 138, and the CDR-H3 comprises the sequence of SEQ ID NO: 246;
31) the CDR-H1 comprises the sequence of SEQ ID NO: 31, the CDR-H2 comprises the sequence of SEQ ID NO: 139, and the CDR-H3 comprises the sequence of SEQ ID NO: 247;
32) the CDR-H1 comprises the sequence of SEQ ID NO: 32, the CDR-H2 comprises the sequence of SEQ ID NO: 140, and the CDR-H3 comprises the sequence of SEQ ID NO: 248;
33) the CDR-H1 comprises the sequence of SEQ ID NO: 33, the CDR-H2 comprises the sequence of SEQ ID NO: 141, and the CDR-H3 comprises the sequence of SEQ ID NO: 249;
34) the CDR-H1 comprises the sequence of SEQ ID NO: 34, the CDR-H2 comprises the sequence of SEQ ID NO: 142, and the CDR-H3 comprises the sequence of SEQ ID NO: 250;
35) the CDR-H1 comprises the sequence of SEQ ID NO: 35, the CDR-H2 comprises the sequence of SEQ ID NO: 143, and the CDR-H3 comprises the sequence of SEQ ID NO: 251;
36) the CDR-H1 comprises the sequence of SEQ ID NO: 36, the CDR-H2 comprises the sequence of SEQ ID NO: 144, and the CDR-H3 comprises the sequence of SEQ ID NO: 252;
37) the CDR-H1 comprises the sequence of SEQ ID NO: 37, the CDR-H2 comprises the sequence of SEQ ID NO: 145, and the CDR-H3 comprises the sequence of SEQ ID NO: 253;
38) the CDR-H1 comprises the sequence of SEQ ID NO: 38, the CDR-H2 comprises the sequence of SEQ ID NO: 146, and the CDR-H3 comprises the sequence of SEQ ID NO: 254;
39) the CDR-H1 comprises the sequence of SEQ ID NO: 39, the CDR-H2 comprises the sequence of SEQ ID NO: 147, and the CDR-H3 comprises the sequence of SEQ ID NO: 255;
40) the CDR-H1 comprises the sequence of SEQ ID NO: 40, the CDR-H2 comprises the sequence of SEQ ID NO: 148, and the CDR-H3 comprises the sequence of SEQ ID NO: 256;
41) the CDR-H1 comprises the sequence of SEQ ID NO: 41, the CDR-H2 comprises the sequence of SEQ ID NO: 149, and the CDR-H3 comprises the sequence of SEQ ID NO: 257;
42) the CDR-H1 comprises the sequence of SEQ ID NO: 42, the CDR-H2 comprises the sequence of SEQ ID NO: 150, and the CDR-H3 comprises the sequence of SEQ ID NO: 258;
43) the CDR-H1 comprises the sequence of SEQ ID NO: 43, the CDR-H2 comprises the sequence of SEQ ID NO: 151, and the CDR-H3 comprises the sequence of SEQ ID NO: 259;
44) the CDR-H1 comprises the sequence of SEQ ID NO: 44, the CDR-H2 comprises the sequence of SEQ ID NO: 152, and the CDR-H3 comprises the sequence of SEQ ID NO: 260;
45) the CDR-H1 comprises the sequence of SEQ ID NO: 45, the CDR-H2 comprises the sequence of SEQ ID NO: 153, and the CDR-H3 comprises the sequence of SEQ ID NO: 261;
46) the CDR-H1 comprises the sequence of SEQ ID NO: 46, the CDR-H2 comprises the sequence of SEQ ID NO: 154, and the CDR-H3 comprises the sequence of SEQ ID NO: 262;
47) the CDR-H1 comprises the sequence of SEQ ID NO: 47, the CDR-H2 comprises the sequence of SEQ ID NO: 155, and the CDR-H3 comprises the sequence of SEQ ID NO: 263;
48) the CDR-H1 comprises the sequence of SEQ ID NO: 48, the CDR-H2 comprises the sequence of SEQ ID NO: 156, and the CDR-H3 comprises the sequence of SEQ ID NO: 264;
49) the CDR-H1 comprises the sequence of SEQ ID NO: 49, the CDR-H2 comprises the sequence of SEQ ID NO: 157, and the CDR-H3 comprises the sequence of SEQ ID NO: 265;
50) the CDR-H1 comprises the sequence of SEQ ID NO: 50, the CDR-H2 comprises the sequence of SEQ ID NO: 158, and the CDR-H3 comprises the sequence of SEQ ID NO: 266;
51) the CDR-H1 comprises the sequence of SEQ ID NO: 51, the CDR-H2 comprises the sequence of SEQ ID NO: 159, and the CDR-H3 comprises the sequence of SEQ ID NO: 267;
52) the CDR-H1 comprises the sequence of SEQ ID NO: 52, the CDR-H2 comprises the sequence of SEQ ID NO: 160, and the CDR-H3 comprises the sequence of SEQ ID NO: 268;
53) the CDR-H1 comprises the sequence of SEQ ID NO: 53, the CDR-H2 comprises the sequence of SEQ ID NO: 161, and the CDR-H3 comprises the sequence of SEQ ID NO: 269;
54) the CDR-H1 comprises the sequence of SEQ ID NO: 54, the CDR-H2 comprises the sequence of SEQ ID NO: 162, and the CDR-H3 comprises the sequence of SEQ ID NO: 270;
55) the CDR-H1 comprises the sequence of SEQ ID NO: 55, the CDR-H2 comprises the sequence of SEQ ID NO: 163, and the CDR-H3 comprises the sequence of SEQ ID NO: 271;
56) the CDR-H1 comprises the sequence of SEQ ID NO: 56, the CDR-H2 comprises the sequence of SEQ ID NO: 164, and the CDR-H3 comprises the sequence of SEQ ID NO: 272;
57) the CDR-H1 comprises the sequence of SEQ ID NO: 57, the CDR-H2 comprises the sequence of SEQ ID NO: 165, and the CDR-H3 comprises the sequence of SEQ ID NO: 273;
58) the CDR-H1 comprises the sequence of SEQ ID NO: 58, the CDR-H2 comprises the sequence of SEQ ID NO: 166, and the CDR-H3 comprises the sequence of SEQ ID NO: 274;
59) the CDR-H1 comprises the sequence of SEQ ID NO: 59, the CDR-H2 comprises the sequence of SEQ ID NO: 167, and the CDR-H3 comprises the sequence of SEQ ID NO: 275;
60) the CDR-H1 comprises the sequence of SEQ ID NO: 60, the CDR-H2 comprises the sequence of SEQ ID NO: 168, and the CDR-H3 comprises the sequence of SEQ ID NO: 276;
61) the CDR-H1 comprises the sequence of SEQ ID NO: 61, the CDR-H2 comprises the sequence of SEQ ID NO: 169, and the CDR-H3 comprises the sequence of SEQ ID NO: 277;
62) the CDR-H1 comprises the sequence of SEQ ID NO: 62, the CDR-H2 comprises the sequence of SEQ ID NO: 170, and the CDR-H3 comprises the sequence of SEQ ID NO: 278;
63) the CDR-H1 comprises the sequence of SEQ ID NO: 63, the CDR-H2 comprises the sequence of SEQ ID NO: 171, and the CDR-H3 comprises the sequence of SEQ ID NO: 279;
64) the CDR-H1 comprises the sequence of SEQ ID NO: 64, the CDR-H2 comprises the sequence of SEQ ID NO: 172, and the CDR-H3 comprises the sequence of SEQ ID NO: 280;
65) the CDR-H1 comprises the sequence of SEQ ID NO: 65, the CDR-H2 comprises the sequence of SEQ ID NO: 173, and the CDR-H3 comprises the sequence of SEQ ID NO: 281;
66) the CDR-H1 comprises the sequence of SEQ ID NO: 66, the CDR-H2 comprises the sequence of SEQ ID NO: 174, and the CDR-H3 comprises the sequence of SEQ ID NO: 282;
67) the CDR-H1 comprises the sequence of SEQ ID NO: 67, the CDR-H2 comprises the sequence of SEQ ID NO: 175, and the CDR-H3 comprises the sequence of SEQ ID NO: 283;
68) the CDR-H1 comprises the sequence of SEQ ID NO: 68, the CDR-H2 comprises the sequence of SEQ ID NO: 176, and the CDR-H3 comprises the sequence of SEQ ID NO: 284;
69) the CDR-H1 comprises the sequence of SEQ ID NO: 69, the CDR-H2 comprises the sequence of SEQ ID NO: 177, and the CDR-H3 comprises the sequence of SEQ ID NO: 285;
70) the CDR-H1 comprises the sequence of SEQ ID NO: 70, the CDR-H2 comprises the sequence of SEQ ID NO: 178, and the CDR-H3 comprises the sequence of SEQ ID NO: 286;
71) the CDR-H1 comprises the sequence of SEQ ID NO: 71, the CDR-H2 comprises the sequence of SEQ ID NO: 179, and the CDR-H3 comprises the sequence of SEQ ID NO: 287;
72) the CDR-H1 comprises the sequence of SEQ ID NO: 72, the CDR-H2 comprises the sequence of SEQ ID NO: 180, and the CDR-H3 comprises the sequence of SEQ ID NO: 288;
73) the CDR-H1 comprises the sequence of SEQ ID NO: 73, the CDR-H2 comprises the sequence of SEQ ID NO: 181, and the CDR-H3 comprises the sequence of SEQ ID NO: 289;
74) the CDR-H1 comprises the sequence of SEQ ID NO: 74, the CDR-H2 comprises the sequence of SEQ ID NO: 182, and the CDR-H3 comprises the sequence of SEQ ID NO: 290;
75) the CDR-H1 comprises the sequence of SEQ ID NO: 75, the CDR-H2 comprises the sequence of SEQ ID NO: 183, and the CDR-H3 comprises the sequence of SEQ ID NO: 291;
76) the CDR-H1 comprises the sequence of SEQ ID NO: 76, the CDR-H2 comprises the sequence of SEQ ID NO: 184, and the CDR-H3 comprises the sequence of SEQ ID NO: 292;
77) the CDR-H1 comprises the sequence of SEQ ID NO: 77, the CDR-H2 comprises the sequence of SEQ ID NO: 185, and the CDR-H3 comprises the sequence of SEQ ID NO: 293;
78) the CDR-H1 comprises the sequence of SEQ ID NO: 78, the CDR-H2 comprises the sequence of SEQ ID NO: 186, and the CDR-H3 comprises the sequence of SEQ ID NO: 294;
79) the CDR-H1 comprises the sequence of SEQ ID NO: 79, the CDR-H2 comprises the sequence of SEQ ID NO: 187, and the CDR-H3 comprises the sequence of SEQ ID NO: 295;
80) the CDR-H1 comprises the sequence of SEQ ID NO: 80, the CDR-H2 comprises the sequence of SEQ ID NO: 188, and the CDR-H3 comprises the sequence of SEQ ID NO: 296;
81) the CDR-H1 comprises the sequence of SEQ ID NO: 81, the CDR-H2 comprises the sequence of SEQ ID NO: 189, and the CDR-H3 comprises the sequence of SEQ ID NO: 297;
82) the CDR-H1 comprises the sequence of SEQ ID NO: 82, the CDR-H2 comprises the sequence of SEQ ID NO: 190, and the CDR-H3 comprises the sequence of SEQ ID NO: 298;
83) the CDR-H1 comprises the sequence of SEQ ID NO: 83, the CDR-H2 comprises the sequence of SEQ ID NO: 191, and the CDR-H3 comprises the sequence of SEQ ID NO: 299;
84) the CDR-H1 comprises the sequence of SEQ ID NO: 84, the CDR-H2 comprises the sequence of SEQ ID NO: 192, and the CDR-H3 comprises the sequence of SEQ ID NO: 300;
85) the CDR-H1 comprises the sequence of SEQ ID NO: 85, the CDR-H2 comprises the sequence of SEQ ID NO: 193, and the CDR-H3 comprises the sequence of SEQ ID NO: 301;
86) the CDR-H1 comprises the sequence of SEQ ID NO: 86, the CDR-H2 comprises the sequence of SEQ ID NO: 194, and the CDR-H3 comprises the sequence of SEQ ID NO: 302;
87) the CDR-H1 comprises the sequence of SEQ ID NO: 87, the CDR-H2 comprises the sequence of SEQ ID NO: 195, and the CDR-H3 comprises the sequence of SEQ ID NO: 303;
88) the CDR-H1 comprises the sequence of SEQ ID NO: 88, the CDR-H2 comprises the sequence of SEQ ID NO: 196, and the CDR-H3 comprises the sequence of SEQ ID NO: 304;
89) the CDR-H1 comprises the sequence of SEQ ID NO: 89, the CDR-H2 comprises the sequence of SEQ ID NO: 197, and the CDR-H3 comprises the sequence of SEQ ID NO: 305;
90) the CDR-H1 comprises the sequence of SEQ ID NO: 90, the CDR-H2 comprises the sequence of SEQ ID NO: 198, and the CDR-H3 comprises the sequence of SEQ ID NO: 306;
91) the CDR-H1 comprises the sequence of SEQ ID NO: 91, the CDR-H2 comprises the sequence of SEQ ID NO: 199, and the CDR-H3 comprises the sequence of SEQ ID NO: 307;
92) the CDR-H1 comprises the sequence of SEQ ID NO: 92, the CDR-H2 comprises the sequence of SEQ ID NO: 200, and the CDR-H3 comprises the sequence of SEQ ID NO: 308;
93) the CDR-H1 comprises the sequence of SEQ ID NO: 93, the CDR-H2 comprises the sequence of SEQ ID NO: 201, and the CDR-H3 comprises the sequence of SEQ ID NO: 309;
94) the CDR-H1 comprises the sequence of SEQ ID NO: 94, the CDR-H2 comprises the sequence of SEQ ID NO: 202, and the CDR-H3 comprises the sequence of SEQ ID NO: 310;
95) the CDR-H1 comprises the sequence of SEQ ID NO: 95, the CDR-H2 comprises the sequence of SEQ ID NO: 203, and the CDR-H3 comprises the sequence of SEQ ID NO: 311;
96) the CDR-H1 comprises the sequence of SEQ ID NO: 96, the CDR-H2 comprises the sequence of SEQ ID NO: 204, and the CDR-H3 comprises the sequence of SEQ ID NO: 312;
97) the CDR-H1 comprises the sequence of SEQ ID NO: 97, the CDR-H2 comprises the sequence of SEQ ID NO: 205, and the CDR-H3 comprises the sequence of SEQ ID NO: 313;
98) the CDR-H1 comprises the sequence of SEQ ID NO: 98, the CDR-H2 comprises the sequence of SEQ ID NO: 206, and the CDR-H3 comprises the sequence of SEQ ID NO: 314;
99) the CDR-H1 comprises the sequence of SEQ ID NO: 99, the CDR-H2 comprises the sequence of SEQ ID NO: 207, and the CDR-H3 comprises the sequence of SEQ ID NO: 315;
100) the CDR-H1 comprises the sequence of SEQ ID NO: 100, the CDR-H2 comprises the sequence of SEQ ID NO: 208, and the CDR-H3 comprises the sequence of SEQ ID NO: 316;
101) the CDR-H1 comprises the sequence of SEQ ID NO: 101, the CDR-H2 comprises the sequence of SEQ ID NO: 209, and the CDR-H3 comprises the sequence of SEQ ID NO: 317;
102) the CDR-H1 comprises the sequence of SEQ ID NO: 102, the CDR-H2 comprises the sequence of SEQ ID NO: 210, and the CDR-H3 comprises the sequence of SEQ ID NO: 318;
103) the CDR-H1 comprises the sequence of SEQ ID NO: 103, the CDR-H2 comprises the sequence of SEQ ID NO: 211, and the CDR-H3 comprises the sequence of SEQ ID NO: 319;
104) the CDR-H1 comprises the sequence of SEQ ID NO: 104, the CDR-H2 comprises the sequence of SEQ ID NO: 212, and the CDR-H3 comprises the sequence of SEQ ID NO: 320;
105) the CDR-H1 comprises the sequence of SEQ ID NO: 105, the CDR-H2 comprises the sequence of SEQ ID NO: 213, and the CDR-H3 comprises the sequence of SEQ ID NO: 321;
106) the CDR-H1 comprises the sequence of SEQ ID NO: 106, the CDR-H2 comprises the sequence of SEQ ID NO: 214, and the CDR-H3 comprises the sequence of SEQ ID NO: 322;
107) the CDR-H1 comprises the sequence of SEQ ID NO: 107, the CDR-H2 comprises the sequence of SEQ ID NO: 215, and the CDR-H3 comprises the sequence of SEQ ID NO: 323; or
108) the CDR-H1 comprises the sequence of SEQ ID NO: 108, the CDR-H2 comprises the sequence of SEQ ID NO: 216, and the CDR-H3 comprises the sequence of SEQ ID NO: 324.

3. The FAP binding polypeptide of alternative 1 or 2, wherein the heavy chain variable domain comprises an amino acid sequence having at least 90%, 95%, 99%, or 100% sequence identity to any sequence selected from SEQ ID NOs: 325-432.

4. The FAP binding polypeptide of any one of alternatives 1-3, wherein the FAP binding polypeptide is humanized.

5. The FAP binding polypeptide of any one of alternatives 1-4, wherein the FAP binding polypeptide is a single domain antibody (sdAb).

6. A FAP binding polypeptide comprising an immunoglobulin heavy chain variable domain comprising a CDR-H1, wherein the CDR-H1 comprises a sequence having at least 90%, 95%, 99%, or 100% sequence identity to a sequence selected from SEQ ID NOs: 1-108.

7. The FAP binding polypeptide of alternative 6, wherein the CDR-H1 comprises a sequence having at least 90%, 95%, 99%, or 100% sequence identity to a sequence selected from SEQ ID NO: 4.

8. The FAP binding polypeptide of alternative 6 or 7, wherein the immunoglobulin heavy chain variable domain further comprises a CDR-H2, wherein the CDR-H2 comprises a sequence having at least 90%, 95%, 99%, or 100% sequence identity to a sequence selected from SEQ ID NOs: 109-216.

9. The FAP binding polypeptide of any one of alternatives 6-8, wherein the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324.

10. The FAP binding polypeptide of any one of alternatives 6-9, wherein the immunoglobulin heavy chain variable domain comprises an amino acid sequence having at least 90%, 95%, 99%, or 100% sequence identity to any sequence selected from SEQ ID NOs: 325-432.

11. A FAP binding polypeptide comprising an immunoglobulin heavy chain variable domain comprising a CDR-H2, wherein the CDR-H2 comprises a sequence having at least 90%, 95%, 99%, or 100% sequence identity to a sequence selected from SEQ ID NOs: 109-216.

12. The FAP binding polypeptide of alternative 11, wherein the CDR-H2 comprises a sequence having at least 90%, 95%, 99%, or 100% sequence identity to a sequence selected from SEQ ID NO: 110.

13. The FAP binding polypeptide of alternative 11 or 12, wherein the immunoglobulin heavy chain variable domain further comprises a CDR-H1, wherein the CDR-H1 comprises a sequence having at least 90%, 95%, 99%, or 100% sequence identity to a sequence selected from SEQ ID NOs: 1-108.

14. The FAP binding polypeptide of any one of alternatives 11-13, wherein the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324.

15. The FAP binding polypeptide of any one of alternatives 11-14, wherein the immunoglobulin heavy chain variable domain comprises an amino acid sequence having at least 90%, 95%, 99%, or 100% sequence identity to any sequence selected from SEQ ID NOs: 325-432.

16. A chimeric antigen receptor (CAR) comprising the FAP binding polypeptide of any one of alternatives 1-15.

17. A chimeric antigen receptor (CAR) cell comprising the CAR of alternative 16.

18. The CAR cell of alternative 17, wherein the CAR cell is a CAR T cell.

19. The CAR cell of alternative 17 or 18, wherein the CAR cell comprises at least two binding polypeptides and the CAR cell is a multivalent CAR cell.

20. The CAR cell of any one of alternatives 17-19, wherein the CAR cell is derived from a subject or from a cell line.

21. The CAR cell of alternative 20, wherein the subject has a cancer.

22. The CAR cell of alternative 21, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

23. A nucleic acid that encodes for a polypeptide comprising a sequence having at least 90%, 95%, 99%, or 100% sequence identity to the FAP binding polypeptide of any one of alternatives 1-15 or the CAR of alternative 16.

24. A method of treating a cancer in a subject in need thereof, comprising administering the CAR cell of any one of alternatives 17-22.

25. The method of alternative 24, wherein the chimeric antigen receptor cell is autologous or allogeneic to the subject.

26. The method of alternative 24 or 25, wherein the subject is a mammal.

27. The method of any one of alternatives 24-26, wherein the subject is a human.

28. The method of any one of alternatives 24-27, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

29. The method of any one of alternatives 24-28, wherein the chimeric antigen receptor cell is administered parenterally.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts an exemplary alignment for the heavy chain variable domain CDRs disclosed herein.

### DETAILED DESCRIPTION OF THE DISCLOSURE

Disclosed herein are binding polypeptides that are incorporated into a chimeric antigen receptor cell. In some embodiments, the chimeric antigen receptor cell is a chimeric antigen receptor T cell (CAR-T cell). These CAR-Ts may be constructed through processes conventionally known in the art. The binding polypeptides provide specificity towards their respective tumor-associated antigens, enabling targeting of cancers expressing said tumor-associated antigens by the CAR-T cell.

In some embodiments, the binding polypeptides are single domain antibodies (sdAbs) disposed on the surface of the chimeric antigen receptor cells (e.g. CAR-T cell). The sdAbs may be specific for, or have binding affinity towards, a tumor-associated antigen. In some embodiments, the tumor-associated antigen is fibroblast activation protein (FAP).

Also disclosed herein are methods of treating a cancer in a subject in need thereof by administering a chimeric antigen receptor cell comprising one or more of the binding polypeptides disclosed herein. In some embodiments, the cancer may be breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. The CAR-T cell may be derived from the subject for an autologous treatment. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. Alternatively, the CAR-T cell may be derived from the same species as the subject for an allogeneic treatment.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The term “administering” includes enteral, oral, intranasal, parenteral, intravenous, intraperitoneal, intramuscular, intra-arteriole, intraventricular, intradermal, intralesional, intracranial, intrathecal, or subcutaneous administration, or the implantation of a slow-release device, e.g., a mini-osmotic pump, to a subject.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acid monomers can be linked by phosphodiester bonds or analogs of such linkages. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Those skilled in the art will appreciate that gene expression levels are dependent on many factors, such as promoter sequences and regulatory elements. In this aspect, many synthetic genes can be designed to increase their protein expression level.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

In some embodiments, the nucleic acid or peptide sequences presented herein and used in the examples are functional in various biological systems including but not limited to humans, mice, rats, monkeys, primates, cats, dogs, rabbits, E. coli, yeast, and mammalian cells. In other embodiments, nucleic acid or peptide sequences sharing at least or lower than 0%, 10%, 20%, 30%, 40%, 50%, 60%, 70%, 80%, 90%, 91%, 92%, 93%, 94%, 95%, 96%, 97%, 98%, 99%, or 100% identity, or any percentage within a range defined by any two of the aforementioned percentages of identity to the nucleic acid or peptide sequences presented herein and used in the examples can also be used with little or no effect on the function of the sequences in biological systems. As used herein, the term “identity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% identity can encode for the same polypeptide by comprising different codons that encode for the same amino acid during translation.

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g. hydroxyl, thiol, amine, carboxyl, and the like). The term “% similarity” refers to the percentage of units (i.e. amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g. within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to antibody sequences, these similar substitutions may apply to antigen-binding regions (i.e. CDRs) or regions that do not bind to antigens or are only secondary to antigen binding (i.e. framework regions).

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope.

The term “antibody library” refers to a collection of antibodies and/or antibody fragments displayed for screening and/or combination into full antibodies. The antibodies and/or antibody fragments may be displayed on a ribosome; on a phage; or on a cell surface, in particular a yeast cell surface.

The term “compete,” as used herein with regard to an antibody or binding polypeptide, means that a first antibody or binding polypeptide, or an antigen-binding portion thereof, binds to an epitope in a manner sufficiently similar to the binding of a second antibody or binding polypeptide, or an antigen-binding portion thereof, such that the result of binding of the first antibody or binding polypeptide with its cognate epitope is detectably decreased in the presence of the second antibody or binding polypeptide compared to the binding of the first antibody or binding polypeptide in the absence of the second antibody or binding polypeptide. The alternative, where the binding of the second antibody or binding polypeptide to its epitope is also detectably decreased in the presence of the first antibody or binding polypeptide, can, but need not be the case. Regardless of the mechanism by which such competition occurs (e.g., steric hindrance, conformational change, or binding to a common epitope, or portion thereof), the skilled artisan would appreciate, based upon the teachings provided herein, that such competing antibodies or binding polypeptides are encompassed and can be useful for the methods disclosed herein.

An antibody or binding polypeptide that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody or binding polypeptide “specifically binds” or “preferentially binds” to a target if it binds with greater affinity, and/or avidity, and/or more readily, and/or with greater duration than it binds to other substances.

The term “humanized” as applies to a non-human (e.g. rodent or primate) antibodies are hybrid immunoglobulins, immunoglobulin chains or fragments thereof which contain minimal sequence derived from non-human immunoglobulin.

The term “single domain binding polypeptide” or “single domain antibody” (sdAb) as used herein refers to a single peptide strand (e.g. not bound to another peptide strand with disulfide bonds) comprising an intact immunoglobulin domain or other protein fold which can recognize antigens. Single domain binding polypeptides or sdAbs may be derived from typical heavy or light immunoglobulin chains, such as from human, or from alternative sources such as dromedaries (e.g. V_{H}H) and cartilaginous fish (e.g. V_{NAR}). In some embodiments, the single domain binding polypeptide or sdAb comprises one, two, or three complementarity determining regions (CDRs). In some embodiments, the single domain binding polypeptide or sdAb comprises one, two, or three of a CDR1, CDR2, and CDR3.

The term “single-chain variable fragment” (scFv) as used herein is a fusion protein comprising the variable regions of the heavy (VH) and light chains (VL) of an immunoglobulin, in which the VH and VL are covalently linked to form a VH:VL heterodimer. The VH and VL are either joined directly or joined by a peptide-encoding linker, which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences. In some embodiments, the VH and VL of the scFv each comprises one, two, or three CDRs. In some embodiments, the VH and VL of the scFv each comprises one, two, or three of a CDR1, CDR2, and CDR3.

In certain embodiments, definitive delineation of a CDR and identification of residues comprising the binding site of an antibody or binding polypeptide is accomplished by solving the structure of the antibody or binding polypeptide and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “chimeric antigen receptor (CAR)” as used herein refers to engineered biological receptors that confers an artificial specificity in an immune cell towards a certain antigen, such as a tumor-associated antigen. An exemplary immune cell in which CARs can be used are T cells, but it is envisioned that CARs can be engineered into any amenable cytotoxic immune cell, including but not limited to T cells, Natural Killer (NK) cells, Natural Killer T (NKT) cells, dendritic cells, or macrophages. In this aspect, any disclosure pertaining to CAR T cells can also be applied to other immune cells comprising CARs. At their core, CARs comprise an extracellular antigen-recognizing domain (e.g. tumor receptor ligand, or antibody), hinge, transmembrane, and intracellular signaling domain (endodomain). Different combinations of these CAR components may result in different specificities and efficacy against certain cancer antigens.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein refers to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “administering” includes oral administration, topical contact, administration as a suppository, parenteral, intravenous, intraperitoneal, intramuscular, intralesional, intrathecal, intranasal, subdermal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

The term “% w/w” or “% wt/wt” means a percentage expressed in terms of the weight of the ingredient or agent over the total weight of the composition multiplied by 100.

### Antigen Binding Polypeptides

Unless otherwise specified, the complementarity determining regions (CDRs) disclosed herein follow the IMGT definition. However, the CDRs, either separately or within the context of the variable domains, can also be interpreted by Kabat, Chothia, or other definitions as understood by those of skill in the art.

Disclosed herein are fibroblast activation protein (FAP) binding polypeptides. In some embodiments, the FAP binding polypeptides comprise an immunoglobulin heavy chain variable domain comprising a CDR-H1, CDR-H2, and CDR-H3. In some embodiments, the CDR-H1 comprises a sequence having at least 90%, 95%, 99%, or 100% sequence identity to a sequence selected from SEQ ID NOs: 1-108. In some embodiments, CDR-H2 comprises a sequence having at least 90%, 95%, 99%, or 100% sequence identity to a sequence selected from SEQ ID NOs: 109-216. In some embodiments, the CDR-H3 comprises a sequence having at least 90%, 95%, 99%, or 100% sequence identity to a sequence selected from SEQ ID NOs: 217-324. In some embodiments, the CDR-H1 comprises a sequence having at least 90%, 95%, 99%, or 100% sequence identity to a sequence selected from SEQ ID NOs: 1-108; the CDR-H2 comprises a sequence having at least 90%, 95%, 99%, or 100% sequence identity to a sequence selected from SEQ ID NOs: 109-216; and the CDR-H3 comprises a sequence having at least 90%, 95%, 99%, or 100% sequence identity to a sequence selected from SEQ ID NOs: 217-324.

In some embodiments of the FAP binding polypeptides:
1) the CDR-H1 comprises the sequence of SEQ ID NO: 1, the CDR-H2 comprises the sequence of SEQ ID NO: 109, and the CDR-H3 comprises the sequence of SEQ ID NO: 217;
2) the CDR-H1 comprises the sequence of SEQ ID NO: 2, the CDR-H2 comprises the sequence of SEQ ID NO: 110, and the CDR-H3 comprises the sequence of SEQ ID NO: 218;
3) the CDR-H1 comprises the sequence of SEQ ID NO: 3, the CDR-H2 comprises the sequence of SEQ ID NO: 111, and the CDR-H3 comprises the sequence of SEQ ID NO: 219;
4) the CDR-H1 comprises the sequence of SEQ ID NO: 4, the CDR-H2 comprises the sequence of SEQ ID NO: 112, and the CDR-H3 comprises the sequence of SEQ ID NO: 220;
5) the CDR-H1 comprises the sequence of SEQ ID NO: 5, the CDR-H2 comprises the sequence of SEQ ID NO: 113, and the CDR-H3 comprises the sequence of SEQ ID NO: 221;
6) the CDR-H1 comprises the sequence of SEQ ID NO: 6, the CDR-H2 comprises the sequence of SEQ ID NO: 114, and the CDR-H3 comprises the sequence of SEQ ID NO: 222;
7) the CDR-H1 comprises the sequence of SEQ ID NO: 7, the CDR-H2 comprises the sequence of SEQ ID NO: 115, and the CDR-H3 comprises the sequence of SEQ ID NO: 223;
8) the CDR-H1 comprises the sequence of SEQ ID NO: 8, the CDR-H2 comprises the sequence of SEQ ID NO: 116, and the CDR-H3 comprises the sequence of SEQ ID NO: 224;
9) the CDR-H1 comprises the sequence of SEQ ID NO: 9, the CDR-H2 comprises the sequence of SEQ ID NO: 117, and the CDR-H3 comprises the sequence of SEQ ID NO: 225;
10) the CDR-H1 comprises the sequence of SEQ ID NO: 10, the CDR-H2 comprises the sequence of SEQ ID NO: 118, and the CDR-H3 comprises the sequence of SEQ ID NO: 226;
11) the CDR-H1 comprises the sequence of SEQ ID NO: 11, the CDR-H2 comprises the sequence of SEQ ID NO: 119, and the CDR-H3 comprises the sequence of SEQ ID NO: 227;
12) the CDR-H1 comprises the sequence of SEQ ID NO: 12, the CDR-H2 comprises the sequence of SEQ ID NO: 120, and the CDR-H3 comprises the sequence of SEQ ID NO: 228;
13) the CDR-H1 comprises the sequence of SEQ ID NO: 13, the CDR-H2 comprises the sequence of SEQ ID NO: 121, and the CDR-H3 comprises the sequence of SEQ ID NO: 229;
14) the CDR-H1 comprises the sequence of SEQ ID NO: 14, the CDR-H2 comprises the sequence of SEQ ID NO: 122, and the CDR-H3 comprises the sequence of SEQ ID NO: 230;
15) the CDR-H1 comprises the sequence of SEQ ID NO: 15, the CDR-H2 comprises the sequence of SEQ ID NO: 123, and the CDR-H3 comprises the sequence of SEQ ID NO: 231;
16) the CDR-H1 comprises the sequence of SEQ ID NO: 16, the CDR-H2 comprises the sequence of SEQ ID NO: 124, and the CDR-H3 comprises the sequence of SEQ ID NO: 232;
17) the CDR-H1 comprises the sequence of SEQ ID NO: 17, the CDR-H2 comprises the sequence of SEQ ID NO: 125, and the CDR-H3 comprises the sequence of SEQ ID NO: 233;
18) the CDR-H1 comprises the sequence of SEQ ID NO: 18, the CDR-H2 comprises the sequence of SEQ ID NO: 126, and the CDR-H3 comprises the sequence of SEQ ID NO: 234;
19) the CDR-H1 comprises the sequence of SEQ ID NO: 19, the CDR-H2 comprises the sequence of SEQ ID NO: 127, and the CDR-H3 comprises the sequence of SEQ ID NO: 235;
20) the CDR-H1 comprises the sequence of SEQ ID NO: 20, the CDR-H2 comprises the sequence of SEQ ID NO: 128, and the CDR-H3 comprises the sequence of SEQ ID NO: 236;
21) the CDR-H1 comprises the sequence of SEQ ID NO: 21, the CDR-H2 comprises the sequence of SEQ ID NO: 129, and the CDR-H3 comprises the sequence of SEQ ID NO: 237;
22) the CDR-H1 comprises the sequence of SEQ ID NO: 22, the CDR-H2 comprises the sequence of SEQ ID NO: 130, and the CDR-H3 comprises the sequence of SEQ ID NO: 238;
23) the CDR-H1 comprises the sequence of SEQ ID NO: 23, the CDR-H2 comprises the sequence of SEQ ID NO: 131, and the CDR-H3 comprises the sequence of SEQ ID NO: 239;
24) the CDR-H1 comprises the sequence of SEQ ID NO: 24, the CDR-H2 comprises the sequence of SEQ ID NO: 132, and the CDR-H3 comprises the sequence of SEQ ID NO: 240;
25) the CDR-H1 comprises the sequence of SEQ ID NO: 25, the CDR-H2 comprises the sequence of SEQ ID NO: 133, and the CDR-H3 comprises the sequence of SEQ ID NO: 241;
26) the CDR-H1 comprises the sequence of SEQ ID NO: 26, the CDR-H2 comprises the sequence of SEQ ID NO: 134, and the CDR-H3 comprises the sequence of SEQ ID NO: 242;
27) the CDR-H1 comprises the sequence of SEQ ID NO: 27, the CDR-H2 comprises the sequence of SEQ ID NO: 135, and the CDR-H3 comprises the sequence of SEQ ID NO: 243;
28) the CDR-H1 comprises the sequence of SEQ ID NO: 28, the CDR-H2 comprises the sequence of SEQ ID NO: 136, and the CDR-H3 comprises the sequence of SEQ ID NO: 244;
29) the CDR-H1 comprises the sequence of SEQ ID NO: 29, the CDR-H2 comprises the sequence of SEQ ID NO: 137, and the CDR-H3 comprises the sequence of SEQ ID NO: 245;
30) the CDR-H1 comprises the sequence of SEQ ID NO: 30, the CDR-H2 comprises the sequence of SEQ ID NO: 138, and the CDR-H3 comprises the sequence of SEQ ID NO: 246;
31) the CDR-H1 comprises the sequence of SEQ ID NO: 31, the CDR-H2 comprises the sequence of SEQ ID NO: 139, and the CDR-H3 comprises the sequence of SEQ ID NO: 247;
32) the CDR-H1 comprises the sequence of SEQ ID NO: 32, the CDR-H2 comprises the sequence of SEQ ID NO: 140, and the CDR-H3 comprises the sequence of SEQ ID NO: 248;
33) the CDR-H1 comprises the sequence of SEQ ID NO: 33, the CDR-H2 comprises the sequence of SEQ ID NO: 141, and the CDR-H3 comprises the sequence of SEQ ID NO: 249;
34) the CDR-H1 comprises the sequence of SEQ ID NO: 34, the CDR-H2 comprises the sequence of SEQ ID NO: 142, and the CDR-H3 comprises the sequence of SEQ ID NO: 250;
35) the CDR-H1 comprises the sequence of SEQ ID NO: 35, the CDR-H2 comprises the sequence of SEQ ID NO: 143, and the CDR-H3 comprises the sequence of SEQ ID NO: 251;
36) the CDR-H1 comprises the sequence of SEQ ID NO: 36, the CDR-H2 comprises the sequence of SEQ ID NO: 144, and the CDR-H3 comprises the sequence of SEQ ID NO: 252;
37) the CDR-H1 comprises the sequence of SEQ ID NO: 37, the CDR-H2 comprises the sequence of SEQ ID NO: 145, and the CDR-H3 comprises the sequence of SEQ ID NO: 253;
38) the CDR-H1 comprises the sequence of SEQ ID NO: 38, the CDR-H2 comprises the sequence of SEQ ID NO: 146, and the CDR-H3 comprises the sequence of SEQ ID NO: 254;
39) the CDR-H1 comprises the sequence of SEQ ID NO: 39, the CDR-H2 comprises the sequence of SEQ ID NO: 147, and the CDR-H3 comprises the sequence of SEQ ID NO: 255;
40) the CDR-H1 comprises the sequence of SEQ ID NO: 40, the CDR-H2 comprises the sequence of SEQ ID NO: 148, and the CDR-H3 comprises the sequence of SEQ ID NO: 256;
41) the CDR-H1 comprises the sequence of SEQ ID NO: 41, the CDR-H2 comprises the sequence of SEQ ID NO: 149, and the CDR-H3 comprises the sequence of SEQ ID NO: 257;
42) the CDR-H1 comprises the sequence of SEQ ID NO: 42, the CDR-H2 comprises the sequence of SEQ ID NO: 150, and the CDR-H3 comprises the sequence of SEQ ID NO: 258;
43) the CDR-H1 comprises the sequence of SEQ ID NO: 43, the CDR-H2 comprises the sequence of SEQ ID NO: 151, and the CDR-H3 comprises the sequence of SEQ ID NO: 259;
44) the CDR-H1 comprises the sequence of SEQ ID NO: 44, the CDR-H2 comprises the sequence of SEQ ID NO: 152, and the CDR-H3 comprises the sequence of SEQ ID NO: 260;
45) the CDR-H1 comprises the sequence of SEQ ID NO: 45, the CDR-H2 comprises the sequence of SEQ ID NO: 153, and the CDR-H3 comprises the sequence of SEQ ID NO: 261;
46) the CDR-H1 comprises the sequence of SEQ ID NO: 46, the CDR-H2 comprises the sequence of SEQ ID NO: 154, and the CDR-H3 comprises the sequence of SEQ ID NO: 262;
47) the CDR-H1 comprises the sequence of SEQ ID NO: 47, the CDR-H2 comprises the sequence of SEQ ID NO: 155, and the CDR-H3 comprises the sequence of SEQ ID NO: 263;
48) the CDR-H1 comprises the sequence of SEQ ID NO: 48, the CDR-H2 comprises the sequence of SEQ ID NO: 156, and the CDR-H3 comprises the sequence of SEQ ID NO: 264;
49) the CDR-H1 comprises the sequence of SEQ ID NO: 49, the CDR-H2 comprises the sequence of SEQ ID NO: 157, and the CDR-H3 comprises the sequence of SEQ ID NO: 265;
50) the CDR-H1 comprises the sequence of SEQ ID NO: 50, the CDR-H2 comprises the sequence of SEQ ID NO: 158, and the CDR-H3 comprises the sequence of SEQ ID NO: 266;
51) the CDR-H1 comprises the sequence of SEQ ID NO: 51, the CDR-H2 comprises the sequence of SEQ ID NO: 159, and the CDR-H3 comprises the sequence of SEQ ID NO: 267;
52) the CDR-H1 comprises the sequence of SEQ ID NO: 52, the CDR-H2 comprises the sequence of SEQ ID NO: 160, and the CDR-H3 comprises the sequence of SEQ ID NO: 268;
53) the CDR-H1 comprises the sequence of SEQ ID NO: 53, the CDR-H2 comprises the sequence of SEQ ID NO: 161, and the CDR-H3 comprises the sequence of SEQ ID NO: 269;
54) the CDR-H1 comprises the sequence of SEQ ID NO: 54, the CDR-H2 comprises the sequence of SEQ ID NO: 162, and the CDR-H3 comprises the sequence of SEQ ID NO: 270;
55) the CDR-H1 comprises the sequence of SEQ ID NO: 55, the CDR-H2 comprises the sequence of SEQ ID NO: 163, and the CDR-H3 comprises the sequence of SEQ ID NO: 271;
56) the CDR-H1 comprises the sequence of SEQ ID NO: 56, the CDR-H2 comprises the sequence of SEQ ID NO: 164, and the CDR-H3 comprises the sequence of SEQ ID NO: 272;
57) the CDR-H1 comprises the sequence of SEQ ID NO: 57, the CDR-H2 comprises the sequence of SEQ ID NO: 165, and the CDR-H3 comprises the sequence of SEQ ID NO: 273;
58) the CDR-H1 comprises the sequence of SEQ ID NO: 58, the CDR-H2 comprises the sequence of SEQ ID NO: 166, and the CDR-H3 comprises the sequence of SEQ ID NO: 274;
59) the CDR-H1 comprises the sequence of SEQ ID NO: 59, the CDR-H2 comprises the sequence of SEQ ID NO: 167, and the CDR-H3 comprises the sequence of SEQ ID NO: 275;
60) the CDR-H1 comprises the sequence of SEQ ID NO: 60, the CDR-H2 comprises the sequence of SEQ ID NO: 168, and the CDR-H3 comprises the sequence of SEQ ID NO: 276;
61) the CDR-H1 comprises the sequence of SEQ ID NO: 61, the CDR-H2 comprises the sequence of SEQ ID NO: 169, and the CDR-H3 comprises the sequence of SEQ ID NO: 277;
62) the CDR-H1 comprises the sequence of SEQ ID NO: 62, the CDR-H2 comprises the sequence of SEQ ID NO: 170, and the CDR-H3 comprises the sequence of SEQ ID NO: 278;
63) the CDR-H1 comprises the sequence of SEQ ID NO: 63, the CDR-H2 comprises the sequence of SEQ ID NO: 171, and the CDR-H3 comprises the sequence of SEQ ID NO: 279;
64) the CDR-H1 comprises the sequence of SEQ ID NO: 64, the CDR-H2 comprises the sequence of SEQ ID NO: 172, and the CDR-H3 comprises the sequence of SEQ ID NO: 280;
65) the CDR-H1 comprises the sequence of SEQ ID NO: 65, the CDR-H2 comprises the sequence of SEQ ID NO: 173, and the CDR-H3 comprises the sequence of SEQ ID NO: 281;
66) the CDR-H1 comprises the sequence of SEQ ID NO: 66, the CDR-H2 comprises the sequence of SEQ ID NO: 174, and the CDR-H3 comprises the sequence of SEQ ID NO: 282;
67) the CDR-H1 comprises the sequence of SEQ ID NO: 67, the CDR-H2 comprises the sequence of SEQ ID NO: 175, and the CDR-H3 comprises the sequence of SEQ ID NO: 283;
68) the CDR-H1 comprises the sequence of SEQ ID NO: 68, the CDR-H2 comprises the sequence of SEQ ID NO: 176, and the CDR-H3 comprises the sequence of SEQ ID NO: 284;
69) the CDR-H1 comprises the sequence of SEQ ID NO: 69, the CDR-H2 comprises the sequence of SEQ ID NO: 177, and the CDR-H3 comprises the sequence of SEQ ID NO: 285;
70) the CDR-H1 comprises the sequence of SEQ ID NO: 70, the CDR-H2 comprises the sequence of SEQ ID NO: 178, and the CDR-H3 comprises the sequence of SEQ ID NO: 286;
71) the CDR-H1 comprises the sequence of SEQ ID NO: 71, the CDR-H2 comprises the sequence of SEQ ID NO: 179, and the CDR-H3 comprises the sequence of SEQ ID NO: 287;
72) the CDR-H1 comprises the sequence of SEQ ID NO: 72, the CDR-H2 comprises the sequence of SEQ ID NO: 180, and the CDR-H3 comprises the sequence of SEQ ID NO: 288;
73) the CDR-H1 comprises the sequence of SEQ ID NO: 73, the CDR-H2 comprises the sequence of SEQ ID NO: 181, and the CDR-H3 comprises the sequence of SEQ ID NO: 289;
74) the CDR-H1 comprises the sequence of SEQ ID NO: 74, the CDR-H2 comprises the sequence of SEQ ID NO: 182, and the CDR-H3 comprises the sequence of SEQ ID NO: 290;
75) the CDR-H1 comprises the sequence of SEQ ID NO: 75, the CDR-H2 comprises the sequence of SEQ ID NO: 183, and the CDR-H3 comprises the sequence of SEQ ID NO: 291;
76) the CDR-H1 comprises the sequence of SEQ ID NO: 76, the CDR-H2 comprises the sequence of SEQ ID NO: 184, and the CDR-H3 comprises the sequence of SEQ ID NO: 292;
77) the CDR-H1 comprises the sequence of SEQ ID NO: 77, the CDR-H2 comprises the sequence of SEQ ID NO: 185, and the CDR-H3 comprises the sequence of SEQ ID NO: 293;
78) the CDR-H1 comprises the sequence of SEQ ID NO: 78, the CDR-H2 comprises the sequence of SEQ ID NO: 186, and the CDR-H3 comprises the sequence of SEQ ID NO: 294;
79) the CDR-H1 comprises the sequence of SEQ ID NO: 79, the CDR-H2 comprises the sequence of SEQ ID NO: 187, and the CDR-H3 comprises the sequence of SEQ ID NO: 295;
80) the CDR-H1 comprises the sequence of SEQ ID NO: 80, the CDR-H2 comprises the sequence of SEQ ID NO: 188, and the CDR-H3 comprises the sequence of SEQ ID NO: 296;
81) the CDR-H1 comprises the sequence of SEQ ID NO: 81, the CDR-H2 comprises the sequence of SEQ ID NO: 189, and the CDR-H3 comprises the sequence of SEQ ID NO: 297;
82) the CDR-H1 comprises the sequence of SEQ ID NO: 82, the CDR-H2 comprises the sequence of SEQ ID NO: 190, and the CDR-H3 comprises the sequence of SEQ ID NO: 298;
83) the CDR-H1 comprises the sequence of SEQ ID NO: 83, the CDR-H2 comprises the sequence of SEQ ID NO: 191, and the CDR-H3 comprises the sequence of SEQ ID NO: 299;
84) the CDR-H1 comprises the sequence of SEQ ID NO: 84, the CDR-H2 comprises the sequence of SEQ ID NO: 192, and the CDR-H3 comprises the sequence of SEQ ID NO: 300;
85) the CDR-H1 comprises the sequence of SEQ ID NO: 85, the CDR-H2 comprises the sequence of SEQ ID NO: 193, and the CDR-H3 comprises the sequence of SEQ ID NO: 301;
86) the CDR-H1 comprises the sequence of SEQ ID NO: 86, the CDR-H2 comprises the sequence of SEQ ID NO: 194, and the CDR-H3 comprises the sequence of SEQ ID NO: 302;
87) the CDR-H1 comprises the sequence of SEQ ID NO: 87, the CDR-H2 comprises the sequence of SEQ ID NO: 195, and the CDR-H3 comprises the sequence of SEQ ID NO: 303;
88) the CDR-H1 comprises the sequence of SEQ ID NO: 88, the CDR-H2 comprises the sequence of SEQ ID NO: 196, and the CDR-H3 comprises the sequence of SEQ ID NO: 304;
89) the CDR-H1 comprises the sequence of SEQ ID NO: 89, the CDR-H2 comprises the sequence of SEQ ID NO: 197, and the CDR-H3 comprises the sequence of SEQ ID NO: 305;
90) the CDR-H1 comprises the sequence of SEQ ID NO: 90, the CDR-H2 comprises the sequence of SEQ ID NO: 198, and the CDR-H3 comprises the sequence of SEQ ID NO: 306;
91) the CDR-H1 comprises the sequence of SEQ ID NO: 91, the CDR-H2 comprises the sequence of SEQ ID NO: 199, and the CDR-H3 comprises the sequence of SEQ ID NO: 307;
92) the CDR-H1 comprises the sequence of SEQ ID NO: 92, the CDR-H2 comprises the sequence of SEQ ID NO: 200, and the CDR-H3 comprises the sequence of SEQ ID NO: 308;
93) the CDR-H1 comprises the sequence of SEQ ID NO: 93, the CDR-H2 comprises the sequence of SEQ ID NO: 201, and the CDR-H3 comprises the sequence of SEQ ID NO: 309;
94) the CDR-H1 comprises the sequence of SEQ ID NO: 94, the CDR-H2 comprises the sequence of SEQ ID NO: 202, and the CDR-H3 comprises the sequence of SEQ ID NO: 310;
95) the CDR-H1 comprises the sequence of SEQ ID NO: 95, the CDR-H2 comprises the sequence of SEQ ID NO: 203, and the CDR-H3 comprises the sequence of SEQ ID NO: 311;
96) the CDR-H1 comprises the sequence of SEQ ID NO: 96, the CDR-H2 comprises the sequence of SEQ ID NO: 204, and the CDR-H3 comprises the sequence of SEQ ID NO: 312;
97) the CDR-H1 comprises the sequence of SEQ ID NO: 97, the CDR-H2 comprises the sequence of SEQ ID NO: 205, and the CDR-H3 comprises the sequence of SEQ ID NO: 313;
98) the CDR-H1 comprises the sequence of SEQ ID NO: 98, the CDR-H2 comprises the sequence of SEQ ID NO: 206, and the CDR-H3 comprises the sequence of SEQ ID NO: 314;
99) the CDR-H1 comprises the sequence of SEQ ID NO: 99, the CDR-H2 comprises the sequence of SEQ ID NO: 207, and the CDR-H3 comprises the sequence of SEQ ID NO: 315;
100) the CDR-H1 comprises the sequence of SEQ ID NO: 100, the CDR-H2 comprises the sequence of SEQ ID NO: 208, and the CDR-H3 comprises the sequence of SEQ ID NO: 316;
101) the CDR-H1 comprises the sequence of SEQ ID NO: 101, the CDR-H2 comprises the sequence of SEQ ID NO: 209, and the CDR-H3 comprises the sequence of SEQ ID NO: 317;
102) the CDR-H1 comprises the sequence of SEQ ID NO: 102, the CDR-H2 comprises the sequence of SEQ ID NO: 210, and the CDR-H3 comprises the sequence of SEQ ID NO: 318;
103) the CDR-H1 comprises the sequence of SEQ ID NO: 103, the CDR-H2 comprises the sequence of SEQ ID NO: 211, and the CDR-H3 comprises the sequence of SEQ ID NO: 319;
104) the CDR-H1 comprises the sequence of SEQ ID NO: 104, the CDR-H2 comprises the sequence of SEQ ID NO: 212, and the CDR-H3 comprises the sequence of SEQ ID NO: 320;
105) the CDR-H1 comprises the sequence of SEQ ID NO: 105, the CDR-H2 comprises the sequence of SEQ ID NO: 213, and the CDR-H3 comprises the sequence of SEQ ID NO: 321;
106) the CDR-H1 comprises the sequence of SEQ ID NO: 106, the CDR-H2 comprises the sequence of SEQ ID NO: 214, and the CDR-H3 comprises the sequence of SEQ ID NO: 322;
107) the CDR-H1 comprises the sequence of SEQ ID NO: 107, the CDR-H2 comprises the sequence of SEQ ID NO: 215, and the CDR-H3 comprises the sequence of SEQ ID NO: 323; or
108) the CDR-H1 comprises the sequence of SEQ ID NO: 108, the CDR-H2 comprises the sequence of SEQ ID NO: 216, and the CDR-H3 comprises the sequence of SEQ ID NO: 324.

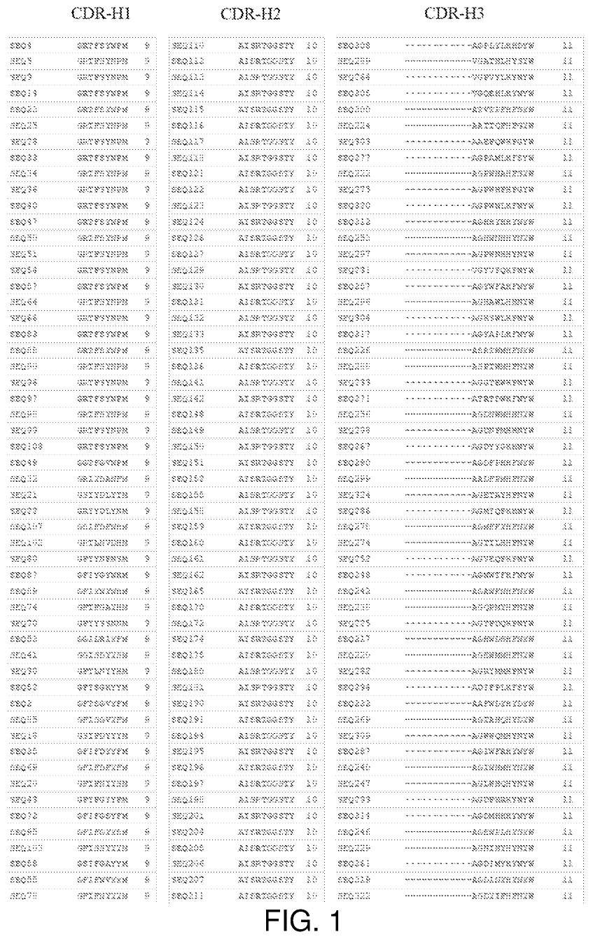

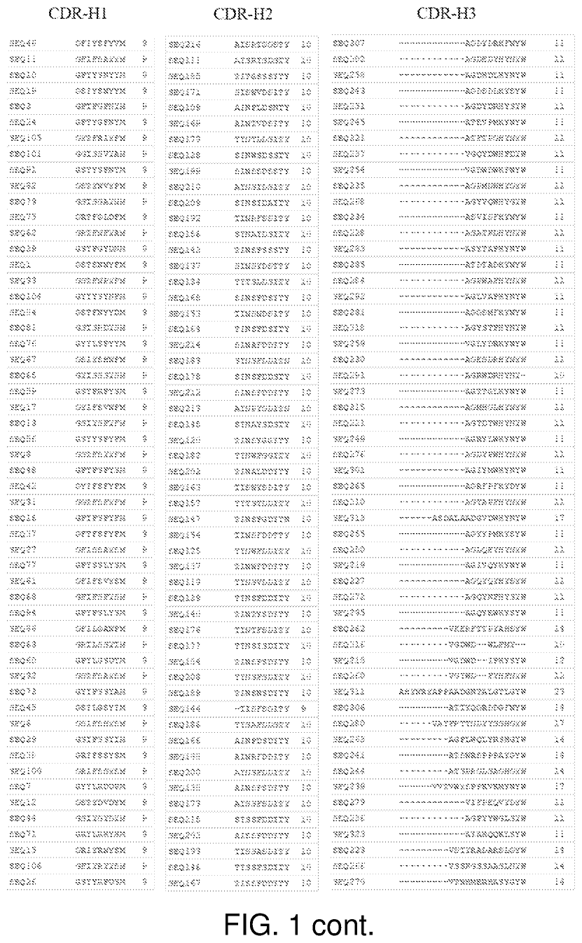

In some embodiments, the FAP binding polypeptide comprise an immunoglobulin heavy chain variable domain comprising a CDR-H1, CDR-H2, and CDR-H3, where one or more of these CDRs are defined by a consensus sequence. The consensus sequences provided herein have been derived from the alignments of CDRs depicted in FIG. 1. However, it is envisioned that alternative alignments may be done (e.g. using global or local alignment, or with different algorithms, such as Hidden Markov Models, seeded guide trees, Needleman-Wunsch algorithm, or Smith-Waterman algorithm, or other known methods) and as such, alternative consensus sequences can be derived (including those done with a subset of the sequences provided herein).

In some embodiments, the CDR-H1 is defined by the formula X₁X₂X₃X₄X₅X₆X₇X₈X₉, where X₁ is G; X₂ is F, G, R, S, or Y; X₃ is I or T; X₄ is F, L, S, or Y; X₅ is D, G, N, R, or S; X₆ is A, D, F, H, I, L, N, P, S, V, or Y; X₇ is D, N, or Y; X₈ is A, D, F, H, I, N, P, S, T, V, or Y; X₉ is M. In some embodiments, the CDR-H1 comprises a sequence having at least 90%, 91%, 92%, 93%, 94%, 95%, 96%, 97%, 98%, 99%, or 100% identity to this consensus sequence. In some embodiments, the CDR-H1 comprises a sequence having 0, 1, 2, 3, 4, 5, or 6 substitutions from this consensus sequence.

In some embodiments, the CDR-H2 is defined by the formula X₁X₂X₃X₄X₅X₆X₇X₈X₉X₁₀, where X₁ is no amino acid, A, S, or T; X₂ is I or T; X₃ is I, N, S, or T; X₄ is A, G, P, R, S, T, or W; X₅ is A, D, F, I, L, N, S, T, V, or Y; X₆ is D, G, or S; X₇ is A, D, G, or S; X₈ is I, N, S, or T; X₉ is T; X₁₀ is Y or N. In some embodiments, the CDR-H2 comprises a sequence having at least 90%, 91%, 92%, 93%, 94%, 95%, 96%, 97%, 98%, 99%, or 100% identity to this consensus sequence. In some embodiments, the CDR-H2 comprises a sequence having 0, 1, 2, 3, 4, 5, or 6 substitutions from this consensus sequence.

In some embodiments, the CDR-H3 is defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃, where X₁ is no amino acid or A; X₂ is no amino acid or H; X₃ is no amino acid or Y; X₄ is no amino acid or N; X₅ is no amino acid or R; X₆ is no amino acid or Y; X₇ is no amino acid, A, or V; X₈ is no amino acid, A, P, S, or V; X₉ is no amino acid, D, P, or T; X₁₀ is no amino acid, A, F, or V; X₁₁ is no amino acid, A, G, K, L, R, S, T, or W; X₁₂ is no amino acid, A, D, E, I, N, P, S, T, or Y; X₁₃ is A, G, H, K, L, P, R, S, T, V, W, or Y; X₁₄ is A, D, F, G, I, M, N, P, Q, R, S, T, or W; X₁₅ is no amino acid, A, D, E, F, G, H, I, L, M, N, P, Q, R, S, T, V, W, or Y; X₁₆ is no amino acid, A, D, E, F, H, I, K, L, M, N, P, Q, R, S, T, V, W, or Y; X₁₇ is A, D, E, F, G, H, I, K, L, M, N, P, Q, S, T, V, W, or Y; X₁₈ is A, D, F, G, H, K, L, M, N, P, Q, R, S, W, or Y; X₁₉ is A, F, G, H, K, M, S, T, or V; X₂₀ is F, H, L, M, or Y; X₂₁ is D, G, N, S, or Y; X₂₂ is no amino acid or Y; X₂₃ is no amino acid or W.

In some embodiments, the CDR-H3 comprises a sequence having at least 90%, 91%, 92%, 93%, 94%, 95%, 96%, 97%, 98%, 99%, or 100% identity to this consensus sequence. In some embodiments, the CDR-H3 comprises a sequence having 0, 1, 2, 3, 4, 5, or 6 substitutions from this consensus sequence.

In some embodiments of the FAP binding polypeptides, the heavy chain variable domain comprises an amino acid sequence having at least 90%, 95%, 99%, or 100% sequence identity to any sequence selected from SEQ ID NOs: 325-432.

Also disclosed herein are FAP binding polypeptides. In some embodiments, the FAP binding polypeptides comprise an immunoglobulin heavy chain variable domain comprising a CDR-H1. In some embodiments, the CDR-H1 comprises a sequence having at least 90%, 95%, 99%, or 100% sequence identity to a sequence selected from SEQ ID NOs: 1-108. In some embodiments, the CDR-H1 comprises a sequence having at least 90%, 95%, 99%, or 100% sequence identity to a sequence selected from SEQ ID NO: 4, or in the alternative, SEQ ID NOs: 5, 9, 14, 23, 25, 28, 33, 34, 36, 40, 47, 50, 51, 54, 57, 64, 66, 83, 88, 90, 96, 97, 98, or 99. In some embodiments, the immunoglobulin heavy chain variable domain further comprises a CDR-H2, wherein the CDR-H2 comprises a sequence having at least 90%, 95%, 99%, or 100% sequence identity to a sequence selected from SEQ ID NOs: 109-216. In some embodiments, the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324. In some embodiments, the immunoglobulin heavy chain variable domain comprises an amino acid sequence having at least 90%, 95%, 99%, or 100% sequence identity to any sequence selected from SEQ ID NOs: 325-432.

Also disclosed herein are FAP binding polypeptides. In some embodiments, the FAP binding polypeptides comprise an immunoglobulin heavy chain variable domain comprising a CDR-H2. In some embodiments, the CDR-H2 comprises a sequence having at least 90%, 95%, 99%, or 100% sequence identity to a sequence selected from SEQ ID NOs: 109-216. In some embodiments, the CDR-H2 comprises a sequence having at least 90%, 95%, 99%, or 100% sequence identity to a sequence selected from SEQ ID NO: 110, or in the alternative, SEQ ID NOs: 112-118, 121-124, 126, 127, 129-133, 135, 136, 141, 142, 148-152, 155, 158-162, 165, 170, 172, 174, 175, 180, 181, 190, 191, 194-198, 201, 204-207, 211, or 216. In some embodiments, the immunoglobulin heavy chain variable domain further comprises a CDR-H1, wherein the CDR-H1 comprises a sequence having at least 90%, 95%, 99%, or 100% sequence identity to a sequence selected from SEQ ID NOs: 1-108. In some embodiments, the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324. In some embodiments, the immunoglobulin heavy chain variable domain comprises an amino acid sequence having at least 90%, 95%, 99%, or 100% sequence identity to any sequence selected from SEQ ID NOs: 325-432.

In some embodiments, the FAP binding polypeptide is humanized. In some embodiments, the FAP binding polypeptide is a single domain antibody (sdAb).

In some embodiments, the FAP binding polypeptide binds to FAP with a dissociation constant (KD) of less than 1 nM, 2 nM, 5 nM, 10 nM, 15 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or 1000 nM, or any KD within a range defined by any two of the aforementioned KD.

The binding polypeptides disclosed herein may be obtained from an antibody library. In some embodiments, the antibody library is an immune antibody library, a naïve antibody library, a synthetic antibody library, or a semi-synthetic antibody library. In some embodiments, the antibody library comprises antibodies derived from human, or antibodies that are not immunogenic in humans, or both. In some embodiments, the antibody library comprises antibodies that are humanized, e.g. from mouse, rat, guinea pig, rabbit, cat, dog, cow, horse, sheep, goat, horse, donkey. In some embodiments, the antibody library comprises single domain antibodies (sdAb), nanobodies, V_{H}H fragments, V_{NAR} fragments, single-chain variable fragments (scFv), camelid antibodies, or cartilaginous fish antibodies, or any combination thereof. One exemplary library that can be used is a fully humanized, synthetic, sdAb library, but any other antibody library that can be prepared or is available can be used for the methods disclosed herein. In some embodiments, the antibody library comprises sdAb. In some embodiments, the antibody library comprises at least 100, 500, 1000, 2000, 3000, 4000, 5000, 6000, 7000, 8000, 9000, 10000, 20000, 30000, 40000, 50000, 60000, 70000, 80000, 90000, 100000, 500000, or 1000000 unique antibodies, or any number of antibodies within a range defined by any two of the aforementioned number of antibodies.

Antibody libraries may be generated computationally or using machine learning processes. An exemplary method of generating an antibody library computationally includes modifying a universal V_{H}H framework with synthetic diversity in one or more complementary determining regions (CDRs), such as CDR1, CDR2, or CDR3, or any combination thereof. The diversity of the CDRs are introduced by randomizing the library of sequences encoding for the antibodies with degenerate codons. For example, an NNK codon library can be employed, where the NNK codon comprises N (25% mix of A/T/C/G) and K (50% mix of T/G). In some embodiments, the NNK codon library is constructed with all possible amino acids, or with some amino acids (e.g. cysteine) and stop codon combinations excluded. Other degenerate codon mixes can be substituted for said NNK codon library with minimal experimentation. In other embodiments, the antibody library can be generated using a trimer codon mix, which improves balanced representation of sense codons while reducing the chance of stop codons, improving efficiency of antibody generation and testing. In some embodiments, artificial intelligence-based prediction can be used to randomize specific binding pockets of the antibodies using available binding models or structure data.

In some embodiments, panning the antibody library comprises screening for the candidate binding polypeptides by phage display, yeast display, bacterial display, ribosome display, or mRNA display, or any combination thereof. In some embodiments, panning the antibody library comprises one or more rounds of selection, wherein the candidate binding polypeptides are selected for specificity towards a cancer-associated antigen (e.g. FAP) or cells or tissues displaying the cancer-associated antigen. In some embodiments, the candidate binding polypeptides are selected under conditions including but not limited to tumor microenvironment-like conditions, immunosuppressive conditions, low or high pH, low or high oxygen concentrations, low or high temperatures, low or high viscosity, or any combination thereof, or for specificity towards modified or derivative forms of the one or more cancer-associated antigens. In some embodiments, the immunosuppressive conditions may comprise the presence of tumor-associated macrophages (TAMs), myeloid-derived suppressor cells (MDSCs), tumor-associated neutrophils (TANs), cancer-associated fibroblasts (CAFs), or other immunosuppressive cells, or the presence of adenosine, or both.

In some embodiments, the chimeric antigen receptor cells are from a cell line (e.g. Jurkat). In some embodiments, the chimeric antigen receptor cells are derived from a subject. In some embodiments, the subject has a cancer. In some embodiments, the subject has a cancer, and that cancer expresses any one or more of the cancer-associated antigens disclosed herein (e.g. FAP). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. In some embodiments, the subject is a mammal, such as a human, cat, dog, mouse, rat, hamster, rodent, cow, pig, horse, goat, sheep, donkey, or monkey. In some embodiments, the subject is a human.

### Chimeric Antigen Receptors (CARs) and Cells

Also disclosed herein are chimeric antigen receptors (CARs) comprising any one or more of the FAP binding polypeptides disclosed herein.

In some embodiments, the CAR comprises at least two binding polypeptides and the CAR is a multivalent CAR. In some embodiments, the CAR comprises two binding polypeptides and the CAR is a bivalent CAR. In some embodiments, the CAR comprises three binding polypeptides and the CAR is a trivalent CAR.

In some embodiments, the CAR further comprises one or more signal peptides, linkers with various lengths and composition, hinges, transmembrane domains, costimulatory domains, signaling domains, cytoplasmic domains, functionality signals, proliferation signals, anti-exhaustion signals, anti-inhibitory receptors, tumor/cancer homing proteins, or regulatory molecules, or any combination thereof. In some embodiments, the hinges comprise CD3ζ, CD4, CD8 or CD28 hinges, or computationally designed synthetic hinges with various lengths. In some embodiments, the transmembrane domains comprise CD3ζ, CD4, CD8 or CD28 transmembrane domains, or computationally designed synthetic transmembrane domains. In some embodiments, the costimulatory domains comprise CD8, CD28, ICOS, 4-1BB, OX40 (CD134), CD27, CD40, CD40L, TLR or other TNFR superfamily member or Ig superfamily member costimulatory domains, or other signaling via cytoplasmic domains of IL-2Rβ, IL-15R-α, MyD88, or CD40 or any other Toll-like receptor or IL-1 receptor signaling pathway members.

In some embodiments, the CARs disclosed herein are constructed by assembling CAR expression constructs from nucleic acids encoding for any one of the binding polypeptides disclosed herein and a mixture of compatible nucleic acids encoding for different CAR modules. In some embodiments, different combinations of CARs are produced for use in a CAR library for screening for CAR efficacy (in vitro or in vivo). In some embodiments, unique CARs are produced separately. In some embodiments, the CARs are specific for one target. In some embodiments, the CARs are specific for at least 1, 2, 3, 4, 5, 6, 7, 8, 9, or 10 targets. In some embodiments, the CARs are bi-specific or tri-specific.

To construct any one of CARs disclosed herein, the nucleic acids encoding for the binding polypeptides identified by panning of the antibody library are assembled into CAR expression constructs with other CAR modules. In some embodiments, the CAR expression constructs are assembled using multi-fragment assembly reactions known in the art. One exemplary method of assembling CAR expression constructs involves using Type IIS restriction enzymes to generate nucleic acid fragments with compatible overhang sequences and ligating the nucleic acid fragments with a ligase. As Type IIS restriction enzymes cleave outside of their recognition sites, multiple compatible nucleic acid fragments may be prepared simultaneously. In other embodiments, the CAR expression constructs can be assembled by overlap extension PCR. It is envisioned that any other method of assembling nucleic acid constructs from more than one nucleic acid fragment can be employed. In some embodiments, the different CAR modules comprise signal peptides, linkers, hinges, transmembrane domains, costimulatory domains, activation domains, signaling domains, cytoplasmic domains, functionality signals, proliferation signals, anti-exhaustion signals, anti-inhibitor receptors, cancer homing proteins, or regulatory molecules, or any combination thereof. Some exemplary hinges comprise CD8 hinge, CD28 hinge, IgG1 hinge, or IgG4 hinge. Some exemplary transmembrane domains comprise CD3ζ transmembrane domain, CD8α transmembrane domain, CD4 transmembrane domain, CD28 transmembrane domain, or ICOS transmembrane domain. Some exemplary costimulatory domains comprise CD8 costimulatory domain, CD28 costimulatory domain, 4-1BB costimulatory domain, OX40 (CD134) costimulatory domain, ICOS costimulatory domain, CD27 costimulatory domain, CD40 costimulatory domain, CD40L costimulatory domain, TLR costimulatory domains, MYD88-CD40 costimulatory domain, or KIR2DS2 costimulatory domain. In some embodiments, the different CAR modules are derived from CD8, CD28, 4-1BB, CD3ζ, or any combination thereof. The CAR may also be modified with various additions, including but not limited to cytokines, chemokines, cytokine receptors, chemokine receptors, antigen receptors or ligands, antibodies, or enzymes.

Also disclosed herein are chimeric antigen receptor (CAR) cells comprising any one of the CARs disclosed herein. In some embodiments, the CAR cell is a CAR-T cell. In some embodiments, the CAR cell is derived from a subject or from a cell line. In some embodiments, the subject has a cancer. In some embodiments, the subject has a cancer, and that cancer expresses any one or more of the cancer-associated antigens disclosed herein (e.g. FAP). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma.

### Nucleic Acids

Also disclosed herein are nucleic acids that encode for a polypeptide. In some embodiments, the polypeptide is a binding polypeptide. In some embodiments, the polypeptide is a single domain binding polypeptide. In some embodiments, the polypeptide is any one of the binding polypeptides disclosed herein. In some embodiments, the polypeptide comprises a sequence having at least 90%, 95%, 99%, or 100% sequence identity to: any one or more of the FAP binding polypeptides disclosed herein. In some embodiments, the polypeptide is any one of the CARs disclosed herein.

Any one of the nucleic acids that encode for a binding polypeptide can be prepared by recombinant DNA technology, synthetic chemistry techniques, or a combination thereof. For example, sequences of nucleic acids encoding for the binding polypeptide may be cloned into an expression vector using standard molecular techniques known in the art. Sequences can be obtained from other vectors encoding the desired protein sequence, from PCR-generated fragments using respective template nucleic acids, or by assembly of synthetic oligonucleotides encoding the desired sequences. In some embodiments, the expression vector may be a CAR expression vector, in which it is provided to an immune cell so that it expresses the CAR. In some embodiments, the expression vector may be an expression vector suited for large scale antibody or binding polypeptide production, from which the peptide products can be isolated for further use.

Expression of binding polypeptides or CARs may be confirmed by nucleic acid or protein assays known in the art. For example, the presence of transcribed mRNA of binding polypeptides or CARs can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes for the binding polypeptides or CARs. Expression of the binding polypeptides or CARs can also be determined by examining the expressed peptide. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Methods of Use or Treatment

Also disclosed herein are methods of treating a cancer in a subject in need thereof. In some embodiments, the methods comprise administering a chimeric antigen receptor cell to the subject. In some embodiments, the methods comprise administering any one of the chimeric antigen receptor cells disclosed herein. In some embodiments, the chimeric antigen receptor cell expresses and/or comprises any one or more of the FAP binding polypeptides disclosed herein. In some embodiments, the chimeric antigen receptor cell is a CAR-T cell. In some embodiments, the chimeric antigen receptor cell is derived from the subject and is autologous to the subject. In some embodiments, the chimeric antigen receptor cell is allogeneic to the subject. In some embodiments, the chimeric antigen receptor cell is from a cell line (e.g. Jurkat). In some embodiments, the subject is a mammal, such as a human, cat, dog, mouse, rat, hamster, rodent, cow, pig, horse, goat, sheep, donkey, or monkey. In some embodiments, the subject is a human. In some embodiments, the subject has a cancer that expresses any one or more of the cancer-associated antigens disclosed herein (e.g. FAP). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. In some embodiments, the chimeric antigen receptor cell is administered parenterally.

In some embodiments, the chimeric antigen receptor cell is administered once per day, twice per day, three times per day or more. In some embodiments, the chimeric antigen receptor cell is administered daily, every day, every alternate day, five days a week, once a week, every other week, two weeks per month, three weeks per month, once a month, twice a month, three times per month, or more. In some embodiments, the immune cell is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The ranges for administration are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Methodology

Plasmid Preparation and Quality Check (QC)

The Maxi Plasmid Purification kit (Zymo, D4203) was used for CAR plasmid preparation. Plasmid concentration and quality was analyzed by Nanodrop (260/280 ratio and 260/230 ratio) and the ToxinSensor Chromogenic LAL Endotoxin Assay (Genescript, L00350). Good-quality plasmid DNA will have an A260/A280 ratio of 1.8-2.0, an A260/A230 ratio greater than 2.0, and less than 0.1 EU/μg of endotoxin.

Plasmid Assembly and QC

All CARs were synthesized by Genewiz. Synthetic single domain antibody genes encoding antibodies which bind to FAP were cloned into the pLenti vector and the construct was confirmed using Sanger sequencing. The CAR constructs contain a signal peptide (e.g. CD8 signal peptide), hinge (e.g. CD8α stalk), transmembrane domain (e.g. CD8 transmembrane domain or CD28 transmembrane domain), and signaling domains (e.g. 4-1BB costimulatory domain or CD3ζ signaling domain) and the anti-FAP sdAb.

To monitor CAR expression levels, the expressed cassette of the CAR plasmids can also be engineered to express fluorescent eGFP+T2A self-cleaving peptide sequences. Alternatively, truncated CD19 or truncated EGFR cassettes can be used to monitor by antibody detection. Human sequences (e.g. CD8, 4-1BB, CD3ζ) are accessible on GenBank.

Virus Production and Titration

To produce the lentivirus with the anti-FAP CAR construct, human embryonic kidney 293T (HEK293T) cells were co-transfected with CAR transgene-encoding pLenti transfer plasmid and one or more necessary packaging plasmids (i.e. encoding for Gag, Pol, Rev, VSVG, and optionally Tat). The supernatants of the HEK293T cultures were collected at either 48 or 72 hours after transfection, centrifuged, and filtered with a 0.45 μm filter.

Virus titration was done in Jurkat cells transduced with diluted lentivirus collections. After 48 hours, transduced Jurkat cells were stained with biotinylated recombinant protein L and phycoerythrin (PE)-conjugated streptavidin, and anti-FAP CAR abundance was measured by flow cytometry.

T Cell Transduction and Expansion

Human PBMCs were isolated from peripheral blood of healthy human donors by density gradient centrifugation with the Lymphoprep reagent (StemCell Technologies). PBMCs were resuspended at 1×10⁶ cells/mL in X-VIVO 15 serum-free hematopoietic medium (Lonza, 04-418QCN) with 10 ng/mL IL-2 (Novoprotein, GMP-CD66) and 10 ng/mL IL-7 (Novoprotein, GMP-CD47). PBMCs were stimulated with 50 ng/mL anti-CD3 antibody (Novoprotein, GMP-A018) for 24 hours. Then, PBMCs were transduced with anti-FAP CAR lentivirus (at MOI of 1-10) and expanded in appropriate flasks for 9-10 days in RPMI 1640 basal medium supplemented with 10% fetal bovine serum. T cells were rested for 24 hours at 37° C. before any assay. CAR surface levels, CD3, and CD4/CD8 ratios (using antibodies from Biolegend, 317344, 301012, and 317412) were measured 12 or 14 days after initial stimulation of PBMCs with the anti-CD3 antibody.

T Cell Cryopreservation and Thawing

After 14 days of CAR-T expansion, anti-FAP CAR-T cells were centrifuged and the supernatant was discarded. The cell pellet was resuspended in chilled CryoStor CS10 (StemCell Technologies, 07930) at a viable cell density of 5×10⁷ cells/mL. Aliquots of cell suspension were dispensed into cryovials. The cryovials were cooled with a 1° C./minute decrease. The frozen cells were transferred to liquid nitrogen.

To thaw, the cells are quickly thawed in a 37° C. water bath with gentle agitation. The thawed cells are transferred to a 50 mL conical tube and washed by adding 20 mL of fresh growth medium dropwise. The cells are centrifuged and resuspended in X-VIVO 15 medium at a cell density of 1×10⁶ cells/mL.

Cytotoxicity Assay

The cytotoxicity of anti-FAP CAR T-cells was determined by standard luciferase-based assays. Briefly, target cells expressing firefly luciferase were co-cultured with CAR-T cells in triplicate at the indicated effector:target ratios using white-walled 96-well plates with 2×10⁴ target cells in a total volume of 100 μL per well in X-VIVO 15 medium. Target cells alone as a control were plated at the same cell density. After 48 hours of co-culture, 100 μL of luciferase substrate (ONE-Glo, Promega) was directly added to each well. Emitted light was detected with a luminescence plate reader.

In a similar luciferase based cytotoxicity assay, target cancer cells that are engineered to overexpress luciferase are prepared in a cell culture medium (such as RPMI) and seeded on to 96 well plates with 5×10⁴ to 5×10⁶ cells in a volume of 100 μL/well, and incubated at 37° C. for 24 hours. Then, CAR T-cells are seeded in corresponding wells with CAR T cells at various effector:target (E:T) ratios (e.g., 5:1, 1:1, and 1:5) in 100 μL/well. The co-cultures are then incubated at 37° C. for 24 or 48 hours. Subsequently, the cells are processed with the Luciferase Assay System (Promega, E1501) according to manufacturer's instructions using a plate reader. Luminescence is read in endpoint mode using a BioTek Synergy H4 hybrid microplate reader for 10 seconds. Percentage of specific lysis was calculated using the luciferase activity of digitonin-treated cells as maximum cell death and untreated cells as spontaneous cell death, using the formula % specific lysis=100%×[(experimental luminescence−spontaneous cell death luminescence)/(maximum cell death luminescence−spontaneous cell death luminescence)].

Additional information regarding exemplary luciferase cytotoxicity assays may be found in Matta H et al. “Development and characterization of a novel luciferase based cytotoxicity assay” Scientific Reports (2018) 8,199, which is hereby expressly incorporated by reference in its entirety.

IFN-γ Evaluation

1×10⁶ per well of anti-FAP CAR T-cells were stimulated with target cells at indicated effector:target cell ratios in 6-well tissue culture plates for 24 hours. Interferon gamma (IFN-7) and IL-2 secretion was quantitated through enzyme-linked immunosorbent assay (ELISA) using the human IL-2 ELISA Kit II (BD, 550611) and human IFN-7 ELISA Kit II (BD, 550612)

CD25/CD69 Assay

1×10⁶ per well of anti-FAP CAR T-cells were stimulated with target cells at indicated effector:target cell ratios in 6-well tissue culture plates for 24 hours. Cells were stained with LIVE/DEAD Fixable Near-IR Dead Cell Stain Kit (ThermoFisher) to label dead cells and with anti-CD3, and CAR-T cells were identified as CD3+ GFP+ cells by flow cytometry. CD69 and CD25 on CAR-T cells were stained with Alexa Fluor 700 anti-human CD69 antibody (Biolegend) and PE anti-CD25 antibody (Biolegend).

Sequencing of CAR T Cell Libraries

Cells were collected from anti-FAP CAR-T library screens. Genomic DNA (gDNA) was extracted using the Blood/Cell Genome DNA Mini kit (Tiangen, DP304-03). 75 ng of gDNA was used for PCT to amplify the CAR region of the library. The amplified region was TA cloned with the TA/Blunt-Zero Cloning kit (Vazyme, C601) and sequenced.

### Example 2. Anti-FAP CAR-T Cells for the Treatment of Cancer

A patient presents with a cancer, for example, a cancer that expresses or overexpresses FAP (e.g., such that FAP can be used as a biomarker to detect and target the cancer). In some cases, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some cases, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma.

One or more of the CAR T-cells disclosed herein, which may include any one or more of the anti-FAP CARs and/or anti-FAP binders, are administered to the patient enterally, orally, intranasally, parenterally, intravenously, intraperitoneally, intramuscularly, intra-arterially, intraventricularly, intradermally, intralesionally, intracranially, intrathecally, or subcutaneously.

The CAR T-cells are administered as doses in the amount of 10⁴, 10⁵, 106, 107, 10⁸, or 10⁹ cells per dose, or any number of cells within a range defined by any two of the aforementioned cells per dose, or any number of cells that is effective and/or safe as determined by a trained medical practitioner. The doses are administered every 1, 2, 3, 4, 5, 6, 7, 8, 9, 10, 11, 12, 24, 36, or 48 days, or weeks, or any time within a range defined by any two of the aforementioned times, or any timing of dosing that is effective and/or safe as determined by a trained medical practitioner.

An improvement of the cancer or symptoms thereof is observed in the patient following administration of the one or more CAR T-cells. Administration of the CAR T-cells may be performed in conjunction with another therapy for cancer, including but not limited to immunotherapy, chemotherapy, radiation therapy, surgery, photodynamic therapy, or targeted therapy.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequence List

### FAP Sequences

| | | SEQ | | SEQ | | SEQ |
|---|---|---|---|---|---|---|
| | | ID | | ID | | ID |
| Name | CDR-H1 | NO | CDR-H2 | NO | CDR-H3 | NO |
| FAP- | GSTSNNYFM | 1 | AINPLDSNTY | 109 | AGHWDSHFNYW | 217 |
| FR2-A01 | | | | | | |
| FAP- | GFTSGVYFM | 2 | AISRTGGSTY | 110 | VGDWDIFHYSYW | 218 |
| FR2-B01 | | | | | | |
| FAP- | GFTFGFNYM | 3 | AISRISDSTY | 111 | AGIVQYHYNYW | 219 |
| FR2-C01 | | | | | | |
| FAP- | GRTFSYNPM | 4 | AISRTGGSTY | 112 | AGEWMMHFNYW | 220 |
| FR2-E01 | | | | | | |
| FAP- | GRTFSYNPM | 5 | AISRTGGSTY | 113 | AGTDTWHYNYW | 221 |
| FR2-F01 | | | | | | |
| FAP- | GSIFSHYSM | 6 | AISRTGGSTY | 114 | AGPWHAHFSYW | 222 |
| FR2-G01 | | | | | | |
| FAP- | GYILRDDPM | 7 | AISRTGGSTY | 115 | VSTYRADARSLGYW | 223 |
| FR2-H01 | | | | | | |
| FAP- | GSTFSYYFM | 8 | AISRTGGSTY | 116 | AATTQFHFGYW | 224 |
| FR2-B02 | | | | | | |
| FAP- | GRTFSYNPM | 9 | AISRTGGSTY | 117 | AGTFDQHFNYW | 225 |
| FR2-C02 | | | | | | |
| FAP- | GFIYSNYYM | 10 | AISRTGGSTY | 118 | ASRTWMHFNYW | 226 |
| FR2-D02 | | | | | | |
| FAP- | GFIFSAYYM | 11 | TINSVDDITY | 119 | AGQYQYHYSYW | 227 |
| FR2-E02 | | | | | | |
| FAP- | GSTYDVDYM | 12 | TINSYGGITY | 120 | ASATKDHYNYW | 228 |
| FR2-H02 | | | | | | |
| FAP- | GSIYSFYFM | 13 | AISRTGGSTY | 121 | AGNIHYHYNYW | 229 |
| FR2-B03 | | | | | | |
| FAP- | GRTFSYNPM | 14 | AISRTGGSTY | 122 | AGESDRHYNYW | 230 |
| FR2-C03 | | | | | | |
| FAP- | GRIYRNYSM | 15 | AISRTGGSTY | 123 | VGYVFQHFNYW | 231 |
| FR2-D03 | | | | | | |
| FAP- | GFIFSFYFM | 16 | AISRTGGSTY | 124 | AAFWDYHYDYW | 232 |
| FR2-F03 | | | | | | |
| FAP- | GYIFSVNFM | 17 | TINWFDDITY | 125 | AGGTEWHFNYW | 233 |
| FR2-H03 | | | | | | |
| FAP- | GSIFDYYYM | 18 | AISRTGGSTY | 126 | ASVISFHYNYW | 234 |
| FR2-B04 | | | | | | |
| FAP- | GSIYSNYYM | 19 | AISRTGGSTY | 127 | AGPMNWHYGYW | 235 |
| FR2-D04 | | | | | | |
| FAP- | GFIFNIYSM | 20 | SINWSDSSTY | 128 | AGPYYWGLSYW | 236 |
| FR2-E04 | | | | | | |
| FAP- | GSIYDLYTM | 21 | AISRTGGSTY | 129 | VGQYDWHFDYW | 237 |
| FR2-G04 | | | | | | |
| FAP- | GRIYDLYNM | 22 | AISRTGGSTY | 130 | AGQPMYHFNYW | 238 |
| FR2-A05 | | | | | | |
| FAP- | GRTFSYNPM | 23 | AISRTGGSTY | 131 | VVTVWYSPFKVKMYNYW | 239 |
| FR2-C05 | | | | | | |
| FAP- | GFTYGFNYM | 24 | AISRTGGSTY | 132 | AGIWHMHYNYW | 240 |
| FR2-D05 | | | | | | |
| FAP- | GRTFSYNPM | 25 | AISRTGGSTY | 133 | ATSWRSPPPAYGYW | 241 |
| FR2-E05 | | | | | | |
| FAP- | GSIYRFDSM | 26 | TITSLDSITY | 134 | AGAWKNHFNYW | 242 |
| FR2-F05 | | | | | | |
| FAP- | GFISSAYSM | 27 | AISRTGGSTY | 135 | AGDSDLHYSYW | 243 |
| FR2-G05 | | | | | | |
| FAP- | GRTFSYNPM | 28 | AISRTGGSTY | 136 | ATSPRGLSAGHGYW | 244 |
| FR2-H05 | | | | | | |
| FAP- | GSIFSSYIM | 29 | SINSYDSTTY | 137 | ATEVPMHYNYW | 245 |
| FR2-A06 | | | | | | |
| FAP- | GFTLNIYHM | 30 | AINSFSDITY | 138 | AGEWPLHYSYW | 246 |
| FR2-B06 | | | | | | |
| FAP- | GGTFSFYFM | 31 | TINSFDDITY | 139 | AGLWHQHYNYW | 247 |
| FR2-C06 | | | | | | |
| FAP- | GRIYDANFM | 32 | TINTYSDITY | 140 | AGNWTFHFNYW | 248 |
| FR2-D06 | | | | | | |
| FAP- | GRTFSYNPM | 33 | AISRTGGSTY | 141 | AGRYLWHYNYW | 249 |
| FR2-E06 | | | | | | |
| FAP- | GRTFSYNPM | 34 | AISRTGGSTY | 142 | AGLQEYHYNYW | 250 |
| FR2-F06 | | | | | | |
| FAP- | GFIFDYYFM | 35 | TINSFSSSTY | 143 | AGDYDRHYSYW | 251 |
| FR2-A07 | | | | | | |
| FAP- | GRTFSYNPM | 36 | TISFSGITY | 144 | AGVEQFHFNYW | 252 |
| FR2-B07 | | | | | | |
| FAP- | GFTFSFYFM | 37 | SINAYSDSTY | 145 | AGHWNHHYNYW | 253 |
| FR2-C07 | | | | | | |
| FAP- | GRIFSSYSM | 38 | TISSFSDITY | 146 | VGDWDWHFNYW | 254 |
| FR2-D07 | | | | | | |
| FAP- | GSTFGYDNM | 39 | TINSFGDITN | 147 | AGYYPMHYSYW | 255 |
| FR2-E07 | | | | | | |
| FAP- | GRTFSYNPM | 40 | AISRTGGSTY | 148 | AGDNWMHHNYW | 256 |
| FR2-F07 | | | | | | |
| FAP- | GGISDYYSM | 41 | AISRTGGSTY | 149 | AGYWFAHFNYW | 257 |
| FR2-G07 | | | | | | |
| FAP- | GYIFSFYFM | 42 | AISRTGGSTY | 150 | AGDHDLHYNYW | 258 |
| FR2-H07 | | | | | | |
| FAP- | GFIFGIYFM | 43 | AISRTGGSTY | 151 | VGLYDRHYNYW | 259 |
| FR2-A08 | | | | | | |
| FAP- | GSIYGYDYM | 44 | AISRTGGSTY | 152 | VGIWDFYHFNYW | 260 |
| FR2-C08 | | | | | | |
| FAP- | GSILGSYIM | 45 | TINSNDSITY | 153 | AGDIMYHYNYW | 261 |
| FR2-D08 | | | | | | |
| FAP- | GFIYSFYVM | 46 | TINSFDDTTY | 154 | VKERFTTPYAHSYW | 262 |
| FR2-E08 | | | | | | |
| FAP- | GRTFSYNPM | 47 | AISRTGGSTY | 155 | AGPLWQLYRSHGYW | 263 |
| FR2-G08 | | | | | | |
| FAP- | GFTFSFYSM | 48 | SINAIDSITY | 156 | VGPVYLHYNYW | 264 |
| FR2-A09 | | | | | | |
| FAP- | GGTFGVNPM | 49 | TITSTDDITY | 157 | AGRFPFHYDYW | 265 |
| FR2-B09 | | | | | | |
| FAP- | GRTFSYNPM | 50 | AISRTGGSTY | 158 | VSSKGSSAASLNYW | 266 |
| FR2-C09 | | | | | | |
| FAP- | GRTFSYNPM | 51 | AISRTGGSTY | 159 | AGDYYGHHNYW | 267 |
| FR2-D09 | | | | | | |
| FAP- | GFISGHYYM | 52 | AISRTGGSTY | 160 | AGYVQWHYGYW | 268 |
| FR2-E09 | | | | | | |
| FAP- | GGILRIYFM | 53 | AISRTGGSTY | 161 | AGTAHQHYDYW | 269 |
| FR2-F09 | | | | | | |
| FAP- | GRTFSYNPM | 54 | AISRTGGSTY | 162 | VTNHMERHASYGYW | 270 |
| FR2-G09 | | | | | | |
| FAP- | GFIFNVYYM | 55 | TISWYSDITY | 163 | ATRTDWHFNYW | 271 |
| FR2-H09 | | | | | | |
| FAP- | GSIYSFYFM | 56 | TINSFDSITY | 164 | AGQYNFHYSYW | 272 |
| FR2-B10 | | | | | | |
| FAP- | GRTFSYNPM | 57 | AISRTGGSTY | 165 | AGTTGLHYNYW | 273 |
| FR2-C10 | | | | | | |
| FAP- | GSIFGAYYM | 58 | AINPDSDITY | 166 | AGTILHHFNYW | 274 |
| FR2-D10 | | | | | | |
| FAP- | GSTFRFYSM | 59 | TISSFDDITY | 167 | AGPWHFHFGYW | 275 |
| FR2-E10 | | | | | | |
| FAP- | GFILGSDTM | 60 | SINSFDSITY | 168 | AGDYPWHYNYW | 276 |
| FR2-F10 | | | | | | |
| FAP- | GFIFSVYSM | 61 | AINTVDSITY | 169 | AGPAMLHFSYW | 277 |
| FR2-G10 | | | | | | |
| FAP- | GRTFNFYAM | 62 | AISRTGGSTY | 170 | AGMFKYHFNYW | 278 |
| FR2-H10 | | | | | | |
| FAP- | GRTLSSYTM | 63 | SISWVDSITY | 171 | VIFPEQVYDYW | 279 |
| FR2-A11 | | | | | | |
| FAP- | GRTFSYNPM | 64 | AISRTGGSTY | 172 | VATFRTTNDYYSSHGYW | 280 |
| FR2-C11 | | | | | | |
| FAP- | GYISSSYSM | 65 | AISSFSDITY | 173 | AGGSMFHYNYW | 281 |
| FR2-D11 | | | | | | |
| FAP- | GRTFSYNPM | 66 | AISRTGGSTY | 174 | AGRIMMHFNYW | 282 |
| FR2-F11 | | | | | | |
| FAP- | GSIYSHNFM | 67 | AISRTGGSTY | 175 | ASYTAFHYNYW | 283 |
| FR2-G11 | | | | | | |
| FAP- | GFIFSFYSM | 68 | TINTFSDITY | 176 | AGPWAFHYNYW | 284 |
| FR2-H11 | | | | | | |
| FAP- | GFIFDFYFM | 69 | TINSISDITY | 177 | ATDTADHYNYW | 285 |
| FR2-B12 | | | | | | |
| FAP- | GFIYSSNNM | 70 | SINSFDDSTY | 178 | AGMIQFHHNYW | 286 |
| FR2-C12 | | | | | | |
| FAP- | GRILRHYHM | 71 | TINTLDSITY | 179 | AGIWFRHYNYW | 287 |
| FR2-D12 | | | | | | |
| FAP- | GFIFGSYFM | 72 | AISRTGGSTY | 180 | ASPTWMHFNYW | 288 |
| FR2-F12 | | | | | | |
| FAP- | GYIFSSYAM | 73 | AISRTGGSTY | 181 | VGATHLHYSYW | 289 |
| FR2-G12 | | | | | | |
| FAP- | GFTFGAYHM | 74 | TINWFGGITY | 182 | AGDFPHHFNYW | 290 |
| FR2-H12 | | | | | | |
| FAP- | GRTFGLDFM | 75 | SINSFDDITN | 183 | AGRWDRHYNY | 291 |
| FR3-A01 | | | | | | |
| FAP- | GYILSFYYM | 76 | TINSFSDITY | 184 | AGLVAFHYNYW | 292 |
| FR3-B01 | | | | | | |
| FAP- | GFISSLYSM | 77 | TITGSSSITY | 185 | AGDFHRHYNYW | 293 |
| FR3-E01 | | | | | | |
| FAP- | GFIFNYYYM | 78 | TISAFDDSTY | 186 | ADIFPLHFSYW | 294 |
| FR3-F01 | | | | | | |
| FAP- | GSTSGAYNM | 79 | TINWFDDITY | 187 | AGQYEWHYSYW | 295 |
| FR3-H01 | | | | | | |
| FAP- | GFIYNFNSM | 80 | AINRFDDITY | 188 | AGHAWLHHNYW | 296 |
| FR3-A02 | | | | | | |
| FAP- | GSTSRDYSM | 81 | TINSNSDITY | 189 | AGPWNHHYNYW | 297 |
| FR3-C02 | | | | | | |
| FAP- | GSTYNVYPM | 82 | AISRTGGSTY | 190 | AGDNFMHHNYW | 298 |
| FR3-D02 | | | | | | |
| FAP- | GRTFSYNPM | 83 | AISRTGGSTY | 191 | AADFPMHFNYW | 299 |
| FR3-F02 | | | | | | |
| FAP- | GSTFNYYDM | 84 | TINRFSSITY | 192 | ATVTPFHFSYW | 300 |
| FR3-G02 | | | | | | |
| FAP- | GFISGVYFM | 85 | TISSASDITY | 193 | AGIYMWHYNYW | 301 |
| FR3-A03 | | | | | | |
| FAP- | GFILGANPM | 86 | AISRTGGSTY | 194 | AGDEDYHYNYW | 302 |
| FR3-B03 | | | | | | |
| FAP- | GFIYGYNHM | 87 | AISRTGGSTY | 195 | AAEFQWHFGYW | 303 |
| FR3-C03 | | | | | | |
| FAP- | GRTFSYNPM | 88 | AISRTGGSTY | 196 | AGHSWLHFNYW | 304 |
| FR3-D03 | | | | | | |
| FAP- | GFIYNYNHM | 89 | AISRTGGSTY | 197 | VGQEHLHYNYW | 305 |
| FR3-A04 | | | | | | |
| FAP- | GRTFSYNPM | 90 | AISRTGGSTY | 198 | ATTYQGRDDGFNYW | 306 |
| FR3-B04 | | | | | | |
| FAP- | GSTYSFNTM | 91 | SINSSDSSTY | 199 | AGDYDRHFNYW | 307 |
| FR3-E04 | | | | | | |
| FAP- | GSTFSAYSM | 92 | AINSFDDITY | 200 | AGPLYLHHDYW | 308 |
| FR3-G04 | | | | | | |
| FAP- | GSTFNPYFM | 93 | AISRTGGSTY | 201 | AGWWQMHYNYW | 309 |
| FR3-H04 | | | | | | |
| FAP- | GFIFSLYSM | 94 | TINALDDITY | 202 | AGTAPFHYNYW | 310 |
| FR3-D05 | | | | | | |
| FAP- | GFIFGYYSM | 95 | AISSFDDITY | 203 | AHYNRYAPPAADGNTALGTLGYW | 311 |
| FR3-E05 | | | | | | |
| FAP- | GRTFSYNPM | 96 | AISRTGGSTY | 204 | AGHHYHHYNYW | 312 |
| FR3-G05 | | | | | | |
| FAP- | GRTFSYNPM | 97 | AISRTGGSTY | 205 | ASDALAADGVDWHYNYW | 313 |
| FR3-H05 | | | | | | |
| FAP- | GRTFSYNPM | 98 | AISRTGGSTY | 206 | AGDMHHHYNYW | 314 |
| FR3-C06 | | | | | | |
| FAP- | GRTFSYNPM | 99 | AISRTGGSTY | 207 | AGMHGLHYNYW | 315 |
| FR3-E06 | | | | | | |
| FAP- | GRIFSSYSM | 100 | TINSFSDITY | 208 | VGDWDWLFMY | 316 |
| FR3-F06 | | | | | | |
| FAP- | GGISSVYAM | 101 | SINSIDAITY | 209 | AGYAPLHFNYW | 317 |
| FR3-G06 | | | | | | |
| FAP- | GRTLNVDHM | 102 | AINSIDSITY | 210 | AGYSTFHYNYW | 318 |
| FR3-H06 | | | | | | |
| FAP- | GFISSYYYM | 103 | AISRTGGSTY | 211 | AGDINYHYNYW | 319 |
| FR3-A07 | | | | | | |
| FAP- | GYIYSYNFM | 104 | SINSFDDITY | 212 | AGPWNLHFNYW | 320 |
| FR3-B07 | | | | | | |
| FAP- | GYTFRIYFM | 105 | AINPYGDITN | 213 | ATFTPGHYNYW | 321 |
| FR3-D07 | | | | | | |
| FAP- | GFIYRYYSM | 106 | SINAFDDITY | 214 | AGDYIFHFNYW | 322 |
| FR3-E07 | | | | | | |
| FAP- | GSIFDFNHM | 107 | SISSFDDITY | 215 | AIAHQQKLSYW | 323 |
| FR3-F07 | | | | | | |
| FAP- | GRTFSYNPM | 108 | AISRTGGSTY | 216 | AGETAYHFNYW | 324 |
| FR3-G07 | | | | | | |

| | | SEQ |
|---|---|---|
| | | ID |
| Name | Heavy Chain Variable Domain Sequence | NO: |
| FAP-FR2- | EVQLVESGGGLVQPGGSLRLSCAASGSTSNNYFMGWFRQAPGKGRELVAAINPLD | 325 |
| A01 | SNTYYPDSVEGRFTISRDNAKRMVYLQMNSLRAEDTAVYYCAAGHWDSHFNYW | |
| | GQGTQVTVSS | |
| FAP-FR2- | EVQLVESGGGLVQPGGSLRLSCAASGFTSGVYFMGWFRQAPGKGRELVAAISRTG | 326 |
| B01 | GSTYYPDSVEGRFTISRDNAKRMVYLQMNSLRAEDTAVYYCAVGDWDIFHYSYW | |
| | GQGTQVTVSS | |
| FAP-FR2- | EVQLVESGGGLVQPGGSLRLSCAASGFTFGFNYMGWFRQAPGKGRELVAAISRIS | 327 |
| C01 | DSTYYPDSVEGRFTISRDNAKRMVYLQMNSLRAEDTAVYYCAAGIVQYHYNYW | |
| | GQGTQVTVSS | |
| FAP-FR2- | EVQLVESGGGLVQPGGSLRLSCAASGRTFSYNPMGWFRQAPGKGRELVAAISRTG | 328 |
| E01 | GSTYYPDSVEGRFTISRDNAKRMVYLQMNSLRAEDTAVYYCAAGEWMMHFNY | |
| | WGQGTQVTVSS | |
| FAP-FR2- | EVQLVESGGGLVQPGGSLRLSCAASGRTFSYNPMGWFRQAPGKGRELVAAISRTG | 329 |
| F01 | GSTYYPDSVEGRFTISRDNAKRMVYLQMNSLRAEDTAVYYCAAGTDTWHYNYW | |
| | GQGTQVTVSS | |
| FAP-FR2- | EVQLVESGGGLVQPGGSLRLSCAASGSIFSHYSMGWFRQAPGKGRELVAAISRTG | 330 |
| G01 | GSTYYPDSVEGRFTISRDNAKRMVYLQMNSLRAEDTAVYYCAAGPWHAHFSYW | |
| | GQGTQVTVSS | |
| FAP-FR2- | EVQLVESGGGLVQPGGSLRLSCAASGYILRDDPMGWFRQAPGKGRELVAAISRTG | 331 |
| H01 | GSTYYPDSVEGRFTISRDNAKRMVYLQMNSLRAEDTAVYYCAVSTYRADARSLG | |
| | YWGQGTQVTVSS | |
| FAP-FR2- | EVQLVESGGGLVQPGGSLRLSCAASGSTFSYYFMGWFRQAPGKGRELVAAISRTG | 332 |
| B02 | GSTYYPDSVEGRFTISRDNAKRMVYLQMNSLRAEDTAVYYCAAATTQFHFGYWG | |
| | QGTQVTVSS | |
| FAP-FR2- | EVQLVESGGGLVQPGGSLRLSCAASGRTFSYNPMGWFRQAPGKGRELVAAISRTG | 333 |
| C02 | GSTYYPDSVEGRFTISRDNAKRMVYLQMNSLRAEDTAVYYCAAGTFDQHFNYW | |
| | GQGTQVTVSS | |
| FAP-FR2- | EVQLVESGGGLVQPGGSLRLSCAASGFIYSNYYMGWFRQAPGKGRELVAAISRTG | 334 |
| D02 | GSTYYPDSVEGRFTISRDNAKRMVYLQMNSLRAEDTAVYYCAASRTWMHFNYW | |
| | GQGTQVTVSS | |
| FAP-FR2- | EVQLVESGGGLVQPGGSLRLSCAASGFIFSAYYMGWFRQAPGKGRELVATINSVD | 335 |
| E02 | DITYYPDSVEGRFTISRDNAKRMVYLQMNSLRAEDTAVYYCAAGQYQYHYSYW | |
| | GQGTQVTVSS | |
| FAP-FR2- | EVQLVESGGGLVQPGGSLRLSCAASGSTYDVDYMGWFRQAPGKGRELVATINSY | 336 |
| H02 | GGITYYPDSVEGRFTISRDNAKRMVYLQMNSLRAEDTAVYYCAASATKDHYNYW | |
| | GQGTQVTVSS | |
| FAP-FR2- | EVQLVESGGGLVQPGGSLRLSCAASGSIYSFYFMGWFRQAPGKGRELVAAISRTG | 337 |
| B03 | GSTYYPDSVEGRFTISRDNAKRMVYLQMNSLRAEDTAVYYCAAGNIHYHYNYW | |
| | GQGTQVTVSS | |
| FAP-FR2- | EVQLVESGGGLVQPGGSLRLSCAASGRTFSYNPMGWFRQAPGKGRELVAAISRTG | 338 |
| C03 | GSTYYPDSVEGRFTISRDNAKRMVYLQMNSLRAEDTAVYYCAAGESDRHYNYW | |
| | GQGTQVTVSS | |
| FAP-FR2- | EVQLVESGGGLVQPGGSLRLSCAASGRIYRNYSMGWFRQAPGKGRELVAAISRTG | 339 |
| D03 | GSTYYPDSVEGRFTISRDNAKRMVYLQMNSLRAEDTAVYYCAVGYVFQHFNYW | |
| | GQGTQVTVSS | |
| FAP-FR2- | EVQLVESGGGLVQPGGSLRLSCAASGFIFSFYFMGWFRQAPGKGRELVAAISRTGG | 340 |
| F03 | STYYPDSVEGRFTISRDNAKRMVYLQMNSLRAEDTAVYYCAAAFWDYHYDYWG | |
| | QGTQVTVSS | |
| FAP-FR2- | EVQLVESGGGLVQPGGSLRLSCAASGYIFSVNFMGWFRQAPGKGRELVATINWFD | 341 |
| H03 | DITYYPDSVEGRFTISRDNAKRMVYLQMNSLRAEDTAVYYCAAGGTEWHENYW | |
| | GQGTQVTVSS | |
| FAP-FR2- | EVQLVESGGGLVQPGGSLRLSCAASGSIFDYYYMGWFRQAPGKGRELVAAISRTG | 342 |
| B04 | GSTYYPDSVEGRFTISRDNAKRMVYLQMNSLRAEDTAVYYCAASVISFHYNYWG | |
| | QGTQVTVSS | |
| FAP-FR2- | EVQLVESGGGLVQPGGSLRLSCAASGSIYSNYYMGWFRQAPGKGRELVAAISRTG | 343 |
| D04 | GSTYYPDSVEGRFTISRDNAKRMVYLQMNSLRAEDTAVYYCAAGPMNWHYGYW | |
| | GQGTQVTVSS | |
| FAP-FR2- | EVQLVESGGGLVQPGGSLRLSCAASGFIFNIYSMGWFRQAPGKGRELVASINWSDS | 344 |
| E04 | STYYPDSVEGRFTISRDNAKRMVYLQMNSLRAEDTAVYYCAAGPYYWGLSYWG | |
| | QGTQVTVSS | |
| FAP-FR2- | EVQLVESGGGLVQPGGSLRLSCAASGSIYDLYTMGWFRQAPGKGRELVAAISRTG | 345 |
| G04 | GSTYYPDSVEGRFTISRDNAKRMVYLQMNSLRAEDTAVYYCAVGQYDWHFDYW | |
| | GQGTQVTVSS | |
| FAP-FR2- | EVQLVESGGGLVQPGGSLRLSCAASGRIYDLYNMGWFRQAPGKGRELVAAISRTG | 346 |
| A05 | GSTYYPDSVEGRFTISRDNAKRMVYLQMNSLRAEDTAVYYCAAGQPMYHFNYW | |
| | GQGTQVTVSS | |
| FAP-FR2- | EVQLVESGGGLVQPGGSLRLSCAASGRTFSYNPMGWFRQAPGKGRELVAAISRTG | 347 |
| C05 | GSTYYPDSVEGRFTISRDNAKRMVYLQMNSLRAEDTAVYYCAVVTVWYSPFKVK | |
| | MYNYWGQGTQVTVSS | |
| FAP-FR2- | EVQLVESGGGLVQPGGSLRLSCAASGFTYGFNYMGWFRQAPGKGRELVAAISRT | 348 |
| D05 | GGSTYYPDSVEGRFTISRDNAKRMVYLQMNSLRAEDTAVYYCAAGIWHMHYNY | |
| | WGQGTQVTVSS | |
| FAP-FR2- | EVQLVESGGGLVQPGGSLRLSCAASGRTFSYNPMGWFRQAPGKGRELVAAISRTG | 349 |
| E05 | GSTYYPDSVEGRFTISRDNAKRMVYLQMNSLRAEDTAVYYCAATSWRSPPPAYG | |
| | YWGQGTQVTVSS | |
| FAP-FR2- | EVQLVESGGGLVQPGGSLRLSCAASGSIYRFDSMGWFRQAPGKGRELVATITSLDS | 350 |
| F05 | ITYYPDSVEGRFTISRDNAKRMVYLQMNSLRAEDTAVYYCAAGAWKNHFNYWG | |
| | QGTQVTVSS | |
| FAP-FR2- | EVQLVESGGGLVQPGGSLRLSCAASGFISSAYSMGWFRQAPGKGRELVAAISRTG | 351 |
| G05 | GSTYYPDSVEGRFTISRDNAKRMVYLQMNSLRAEDTAVYYCAAGDSDLHYSYW | |
| | GQGTQVTVSS | |
| FAP-FR2- | EVQLVESGGGLVQPGGSLRLSCAASGRTFSYNPMGWFRQAPGKGRELVAAISRTG | 352 |
| H05 | GSTYYPDSVEGRFTISRDNAKRMVYLQMNSLRAEDTAVYYCAATSPRGLSAGHG | |
| | YWGQGTQVTVSS | |
| FAP-FR2- | EVQLVESGGGLVQPGGSLRLSCAASGSIFSSYIMGWFRQAPGKGRELVASINSYDS | 353 |
| A06 | TTYYPDSVEGRFTISRDNAKRMVYLQMNSLRAEDTAVYYCAATEVPMHYNYWG | |
| | QGTQVTVSS | |
| FAP-FR2- | EVQLVESGGGLVQPGGSLRLSCAASGFTLNIYHMGWFRQAPGKGRELVAAINSFS | 354 |
| B06 | DITYYPDSVEGRFTISRDNAKRMVYLQMNSLRAEDTAVYYCAAGEWPLHYSYWG | |
| | QGTQVTVSS | |
| FAP-FR2- | EVQLVESGGGLVQPGGSLRLSCAASGGTFSFYFMGWFRQAPGKGRELVATINSFD | 355 |
| C06 | DITYYPDSVEGRFTISRDNAKRMVYLQMNSLRAEDTAVYYCAAGLWHQHYNYW | |
| | GQGTQVTVSS | |
| FAP-FR2- | EVQLVESGGGLVQPGGSLRLSCAASGRIYDANFMGWFRQAPGKGRELVATINTYS | 356 |
| D06 | DITYYPDSVEGRFTISRDNAKRMVYLQMNSLRAEDTAVYYCAAGNWTFHFNYW | |
| | GQGTQVTVSS | |
| FAP-FR2- | EVQLVESGGGLVQPGGSLRLSCAASGRTFSYNPMGWFRQAPGKGRELVAAISRTG | 357 |
| E06 | GSTYYPDSVEGRFTISRDNAKRMVYLQMNSLRAEDTAVYYCAAGRYLWHYNYW | |
| | GQGTQVTVSS | |
| FAP-FR2- | EVQLVESGGGLVQPGGSLRLSCAASGRTFSYNPMGWFRQAPGKGRELVAAISRTG | 358 |
| F06 | GSTYYPDSVEGRFTISRDNAKRMVYLQMNSLRAEDTAVYYCAAGLQEYHYNYW | |
| | GQGTQVTVSS | |
| FAP-FR2- | EVQLVESGGGLVQPGGSLRLSCAASGFIFDYYFMGWFRQAPGKGRELVATINSFSS | 359 |
| A07 | STYYPDSVEGRFTISRDNAKRMVYLQMNSLRAEDTAVYYCAAGDYDRHYSYWG | |
| | QGTQVTVSS | |
| FAP-FR2- | EVQLVESGGGLVQPGGSLRLSCAASGRTFSYNPMGWFRQAPGKGRELVATISFSGI | 360 |
| B07 | TYYPDSVEGRFTISRDNAKRMVYLQMNSLRAEDTAVYYCAAGVEQFHFNYWGQ | |
| | GTQVTVSS | |
| FAP-FR2- | EVQLVESGGGLVQPGGSLRLSCAASGFTFSFYFMGWFRQAPGKGRELVASINAYS | 361 |
| C07 | DSTYYPDSVEGRFTISRDNAKRMVYLQMNSLRAEDTAVYYCAAGHWNHHYNYW | |
| | GQGTQVTVSS | |
| FAP-FR2- | EVQLVESGGGLVQPGGSLRLSCAASGRIFSSYSMGWFRQAPGKGRELVATISSFS | 362 |
| D07 | DITYYPDSVEGRFTISRDNAKRMVYLQMNSLRAEDTAVYYCAVGDWDWHFNYWG | |
| | QGTQVTVSS | |
| FAP-FR2- | EVQLVESGGGLVQPGGSLRLSCAASGSTFGYDNMGWFRQAPGKGRELVATINSFG | 363 |
| E07 | DITNYPDSVEGRFTISRDNAKRMVYLQMNSLRAEDTAVYYCAAGYYPMHYSYW | |
| | GQGTQVTVSS | |
| FAP-FR2- | EVQLVESGGGLVQPGGSLRLSCAASGRTFSYNPMGWFRQAPGKGRELVAAISRTG | 364 |
| F07 | GSTYYPDSVEGRFTISRDNAKRMVYLQMNSLRAEDTAVYYCAAGDNWMHHNY | |
| | WGQGTQVTVSS | |
| FAP-FR2- | EVQLVESGGGLVQPGGSLRLSCAASGGISDYYSMGWFRQAPGKGRELVAAISRTG | 365 |
| G07 | GSTYYPDSVEGRFTISRDNAKRMVYLQMNSLRAEDTAVYYCAAGYWFAHFNYW | |
| | GQGTQVTVSS | |
| FAP-FR2- | EVQLVESGGGLVQPGGSLRLSCAASGYIFSFYFMGWFRQAPGKGRELVAAISRTG | 366 |
| H07 | GSTYYPDSVEGRFTISRDNAKRMVYLQMNSLRAEDTAVYYCAAGDHDLHYNYW | |
| | GQGTQVTVSS | |
| FAP-FR2- | EVQLVESGGGLVQPGGSLRLSCAASGFIFGIYFMGWFRQAPGKGRELVAAISRTGG | 367 |
| A08 | STYYPDSVEGRFTISRDNAKRMVYLQMNSLRAEDTAVYYCAVGLYDRHYNYWG | |
| | QGTQVTVSS | |
| FAP-FR2- | EVQLVESGGGLVQPGGSLRLSCAASGSIYGYDYMGWFRQAPGKGRELVAAISRTG | 368 |
| C08 | GSTYYPDSVEGRFTISRDNAKRMVYLQMNSLRAEDTAVYYCAVGIWDFYHFNYW | |
| | GQGTQVTVSS | |
| FAP-FR2- | EVQLVESGGGLVQPGGSLRLSCAASGSILGSYIMGWFRQAPGKGRELVATINSNDS | 369 |
| D08 | ITYYPDSVEGRFTISRDNAKRMVYLQMNSLRAEDTAVYYCAAGDIMYHYNYWG | |
| | QGTQVTVSS | |
| FAP-FR2- | EVQLVESGGGLVQPGGSLRLSCAASGFIYSFYVMGWFRQAPGKGRELVATINSFD | 370 |
| E08 | DTTYYPDSVEGRFTISRDNAKRMVYLQMNSLRAEDTAVYYCAVKERFTTPYAHS | |
| | YWGQGTQVTVSS | |
| FAP-FR2- | EVQLVESGGGLVQPGGSLRLSCAASGRTFSYNPMGWFRQAPGKGRELVAAISRTG | 371 |
| G08 | GSTYYPDSVEGRFTISRDNAKRMVYLQMNSLRAEDTAVYYCAAGPLWQLYRSHG | |
| | YWGQGTQVTVSS | |
| FAP-FR2- | EVQLVESGGGLVQPGGSLRLSCAASGFTFSFYSMGWFRQAPGKGRELVASINAIDS | 372 |
| A09 | ITYYPDSVEGRFTISRDNAKRMVYLQMNSLRAEDTAVYYCAVGPVYLHYNYWGQ | |
| | GTQVTVSS | |
| FAP-FR2- | EVQLVESGGGLVQPGGSLRLSCAASGGTFGVNPMGWFRQAPGKGRELVATITSTD | 373 |
| B09 | DITYYPDSVEGRFTISRDNAKRMVYLQMNSLRAEDTAVYYCAAGRFPFHYDYWG | |
| | QGTQVTVSS | |
| FAP-FR2- | EVQLVESGGGLVQPGGSLRLSCAASGRTFSYNPMGWFRQAPGKGRELVAAISRTG | 374 |
| C09 | GSTYYPDSVEGRFTISRDNAKRMVYLQMNSLRAEDTAVYYCAVSSKGSSAASLN | |
| | YWGQGTQVTVSS | |
| FAP-FR2- | EVQLVESGGGLVQPGGSLRLSCAASGRTFSYNPMGWFRQAPGKGRELVAAISRTG | 375 |
| D09 | GSTYYPDSVEGRFTISRDNAKRMVYLQMNSLRAEDTAVYYCAAGDYYGHHNYW | |
| | GQGTQVTVSS | |
| FAP-FR2- | EVQLVESGGGLVQPGGSLRLSCAASGFISGHYYMGWFRQAPGKGRELVAAISRTG | 376 |
| E09 | GSTYYPDSVEGRFTISRDNAKRMVYLQMNSLRAEDTAVYYCAAGYVQWHYGYW | |
| | GQGTQVTVSS | |
| FAP-FR2- | EVQLVESGGGLVQPGGSLRLSCAASGGILRIYFMGWFRQAPGKGRELVAAISRTG | 377 |
| F09 | GSTYYPDSVEGRFTISRDNAKRMVYLQMNSLRAEDTAVYYCAAGTAHQHYDYW | |
| | GQGTQVTVSS | |
| FAP-FR2- | EVQLVESGGGLVQPGGSLRLSCAASGRTFSYNPMGWFRQAPGKGRELVAAISRTG | 378 |
| G09 | GSTYYPDSVEGRFTISRDNAKRMVYLQMNSLRAEDTAVYYCAVTNHMERHASYG | |
| | YWGQGTQVTVSS | |
| FAP-FR2- | EVQLVESGGGLVQPGGSLRLSCAASGFIFNVYYMGWFRQAPGKGRELVATISWYS | 379 |
| H09 | DITYYPDSVEGRFTISRDNAKRMVYLQMNSLRAEDTAVYYCAATRTDWHFNYW | |
| | GQGTQVTVSS | |
| FAP-FR2- | EVQLVESGGGLVQPGGSLRLSCAASGSIYSFYFMGWFRQAPGKGRELVATINSFDS | 380 |
| B10 | ITYYPDSVEGRFTISRDNAKRMVYLQMNSLRAEDTAVYYCAAGQYNFHYSYWGQ | |
| | GTQVTVSS | |
| FAP-FR2- | EVQLVESGGGLVQPGGSLRLSCAASGRTFSYNPMGWFRQAPGKGRELVAAISRTG | 381 |
| C10 | GSTYYPDSVEGRFTISRDNAKRMVYLQMNSLRAEDTAVYYCAAGTTGLHYNYW | |
| | GQGTQVTVSS | |
| FAP-FR2- | EVQLVESGGGLVQPGGSLRLSCAASGSIFGAYYMGWFRQAPGKGRELVAAINPDS | 382 |
| D10 | DITYYPDSVEGRFTISRDNAKRMVYLQMNSLRAEDTAVYYCAAGTILHHFNYWG | |
| | QGTQVTVSS | |
| FAP-FR2- | EVQLVESGGGLVQPGGSLRLSCAASGSTFRFYSMGWFRQAPGKGRELVATISSFD | 383 |
| E10 | DITYYPDSVEGRFTISRDNAKRMVYLQMNSLRAEDTAVYYCAAGPWHFHFGYWG | |
| | QGTQVTVSS | |
| FAP-FR2- | EVQLVESGGGLVQPGGSLRLSCAASGFILGSDTMGWFRQAPGKGRELVASINSFDS | 384 |
| F10 | ITYYPDSVEGRFTISRDNAKRMVYLQMNSLRAEDTAVYYCAAGDYPWHYNYWG | |
| | QGTQVTVSS | |
| FAP-FR2- | EVQLVESGGGLVQPGGSLRLSCAASGFIFSVYSMGWFRQAPGKGRELVAAINTVD | 385 |
| G10 | SITYYPDSVEGRFTISRDNAKRMVYLQMNSLRAEDTAVYYCAAGPAMLHFSYWG | |
| | QGTQVTVSS | |
| FAP-FR2- | EVQLVESGGGLVQPGGSLRLSCAASGRTFNFYAMGWFRQAPGKGRELVAAISRTG | 386 |
| H10 | GSTYYPDSVEGRFTISRDNAKRMVYLQMNSLRAEDTAVYYCAAGMFKYHFNYW | |
| | GQGTQVTVSS | |
| FAP-FR2- | EVQLVESGGGLVQPGGSLRLSCAASGRTLSSYTMGWFRQAPGKGRELVASISWVD | 387 |
| A11 | SITYYPDSVEGRFTISRDNAKRMVYLQMNSLRAEDTAVYYCAVIFPEQVYDYWGQ | |
| | GTQVTVSS | |
| FAP-FR2- | EVQLVESGGGLVQPGGSLRLSCAASGRTFSYNPMGWFRQAPGKGRELVAAISRTG | 388 |
| C11 | GSTYYPDSVEGRFTISRDNAKRMVYLQMNSLRAEDTAVYYCAVATFRTTNDYYS | |
| | SHGYWGQGTQVTVSS | |
| FAP-FR2- | EVQLVESGGGLVQPGGSLRLSCAASGYISSSYSMGWFRQAPGKGRELVAAISSFSD | 389 |
| D11 | ITYYPDSVEGRFTISRDNAKRMVYLQMNSLRAEDTAVYYCAAGGSMFHYNYWG | |
| | QGTQVTVSS | |
| FAP-FR2- | EVQLVESGGGLVQPGGSLRLSCAASGRTFSYNPMGWFRQAPGKGRELVAAISRTG | 390 |
| F11 | GSTYYPDSVEGRFTISRDNAKRMVYLQMNSLRAEDTAVYYCAAGRIMMHFNYW | |
| | GQGTQVTVSS | |
| FAP-FR2- | EVQLVESGGGLVQPGGSLRLSCAASGSIYSHNFMGWFRQAPGKGRELVAAISRTG | 391 |
| G11 | GSTYYPDSVEGRFTISRDNAKRMVYLQMNSLRAEDTAVYYCAASYTAFHYNYW | |
| | GQGTQVTVSS | |
| FAP-FR2- | EVQLVESGGGLVQPGGSLRLSCAASGFIFSFYSMGWFRQAPGKGRELVATINTFSD | 392 |
| H11 | ITYYPDSVEGRFTISRDNAKRMVYLQMNSLRAEDTAVYYCAAGPWAFHYNYWG | |
| | QGTQVTVSS | |
| FAP-FR2- | EVQLVESGGGLVQPGGSLRLSCAASGFIFDFYFMGWFRQAPGKGRELVATINSISD | 393 |
| B12 | ITYYPDSVEGRFTISRDNAKRMVYLQMNSLRAEDTAVYYCAATDTADHYNYWGQ | |
| | GTQVTVSS | |
| FAP-FR2- | EVQLVESGGGLVQPGGSLRLSCAASGFIYSSNNMGWFRQAPGKGRELVASINSFD | 394 |
| C12 | DSTYYPDSVEGRFTISRDNAKRMVYLQMNSLRAEDTAVYYCAAGMIQFHHNYW | |
| | GQGTQVTVSS | |
| FAP-FR2- | EVQLVESGGGLVQPGGSLRLSCAASGRILRHYHMGWFRQAPGKGRELVATINTLD | 395 |
| D12 | SITYYPDSVEGRFTISRDNAKRMVYLQMNSLRAEDTAVYYCAAGIWFRHYNYWG | |
| | QGTQVTVSS | |
| FAP-FR2- | EVQLVESGGGLVQPGGSLRLSCAASGFIFGSYFMGWFRQAPGKGRELVAAISRTG | 396 |
| F12 | GSTYYPDSVEGRFTISRDNAKRMVYLQMNSLRAEDTAVYYCAASPTWMHFNYW | |
| | GQGTQVTVSS | |
| FAP-FR2- | EVQLVESGGGLVQPGGSLRLSCAASGYIFSSYAMGWFRQAPGKGRELVAAISRTG | 397 |
| G12 | GSTYYPDSVEGRFTISRDNAKRMVYLQMNSLRAEDTAVYYCAVGATHLHYSYW | |
| | GQGTQVTVSS | |
| FAP-FR2- | EVQLVESGGGLVQPGGSLRLSCAASGFTFGAYHMGWFRQAPGKGRELVATINWF | 398 |
| H12 | GGITYYPDSVEGRFTISRDNAKRMVYLQMNSLRAEDTAVYYCAAGDFPHHFNYW | |
| | GQGTQVTVSS | |
| FAP-FR3- | EVQLVESGGGLVQPGGSLRLSCAASGRTFGLDFMGWFRQAPGKGRELVASINSFD | 399 |
| A01 | DITNYPDSVEGRFTISRDNAKRMVYLQMNSLRAEDTAVYYCAAGRWDRHYNYW | |
| | GQGTQVTVSS | |
| FAP-FR3- | EVQLVESGGGLVQPGGSLRLSCAASGYILSFYYMGWFRQAPGKGRELVATINSFS | 400 |
| B01 | DITYYPDSVEGRFTISRDNAKRMVYLQMNSLRAEDTAVYYCAAGLVAFHYNYWG | |
| | QGTQVTVSS | |
| FAP-FR3- | EVQLVESGGGLVQPGGSLRLSCAASGFISSLYSMGWFRQAPGKGRELVATITGSSS | 401 |
| E01 | ITYYPDSVEGRFTISRDNAKRMVYLQMNSLRAEDTAVYYCAAGDFHRHYNYWGQ | |
| | GTQVTVSS | |
| FAP-FR3- | EVQLVESGGGLVQPGGSLRLSCAASGFIFNYYYMGWFRQAPGKGRELVATISAFD | 402 |
| F01 | DSTYYPDSVEGRFTISRDNAKRMVYLQMNSLRAEDTAVYYCAADIFPLHFSYWG | |
| | QGTQVTVSS | |
| FAP-FR3- | EVQLVESGGGLVQPGGSLRLSCAASGSTSGAYNMGWFRQAPGKGRELVATINWF | 403 |
| HO1 | DDITYYPDSVEGRFTISRDNAKRMVYLQMNSLRAEDTAVYYCAAGQYEWHYSY | |
| | WGQGTQVTVSS | |
| FAP-FR3- | EVQLVESGGGLVQPGGSLRLSCAASGFIYNFNSMGWFRQAPGKGRELVAAINRFD | 404 |
| A02 | DITYYPDSVEGRFTISRDNAKRMVYLQMNSLRAEDTAVYYCAAGHAWLHHNYW | |
| | GQGTQVTVSS | |
| FAP-FR3- | EVQLVESGGGLVQPGGSLRLSCAASGSTSRDYSMGWFRQAPGKGRELVATINSNS | 405 |
| C02 | DITYYPDSVEGRFTISRDNAKRMVYLQMNSLRAEDTAVYYCAAGPWNHHYNYW | |
| | GQGTQVTVSS | |
| FAP-FR3- | EVQLVESGGGLVQPGGSLRLSCAASGSTYNVYPMGWFRQAPGKGRELVAAISRT | 406 |
| D02 | GGSTYYPDSVEGRFTISRDNAKRMVYLQMNSLRAEDTAVYYCAAGDNFMHHNY | |
| | WGQGTQVTVSS | |
| FAP-FR3- | EVQLVESGGGLVQPGGSLRLSCAASGRTFSYNPMGWFRQAPGKGRELVAAISRTG | 407 |
| F02 | GSTYYPDSVEGRFTISRDNAKRMVYLQMNSLRAEDTAVYYCAAADFPMHFNYW | |
| | GQGTQVTVSS | |
| FAP-FR3- | EVQLVESGGGLVQPGGSLRLSCAASGSTFNYYDMGWFRQAPGKGRELVATINRFS | 408 |
| G02 | SITYYPDSVEGRFTISRDNAKRMVYLQMNSLRAEDTAVYYCAATVTPFHFSYWGQ | |
| | GTQVTVSS | |
| FAP-FR3- | EVQLVESGGGLVQPGGSLRLSCAASGFISGVYFMGWFRQAPGKGRELVATISSAS | 409 |
| A03 | DITYYPDSVEGRFTISRDNAKRMVYLQMNSLRAEDTAVYYCAAGIYMWHYNYW | |
| | GQGTQVTVSS | |
| FAP-FR3- | EVQLVESGGGLVQPGGSLRLSCAASGFILGANPMGWFRQAPGKGRELVAAISRTG | 410 |
| B03 | GSTYYPDSVEGRFTISRDNAKRMVYLQMNSLRAEDTAVYYCAAGDEDYHYNYW | |
| | GQGTQVTVSS | |
| FAP-FR3- | EVQLVESGGGLVQPGGSLRLSCAASGFIYGYNHMGWFRQAPGKGRELVAAISRTG | 411 |
| C03 | GSTYYPDSVEGRFTISRDNAKRMVYLQMNSLRAEDTAVYYCAAAEFQWHFGYW | |
| | GQGTQVTVSS | |
| FAP-FR3- | EVQLVESGGGLVQPGGSLRLSCAASGRTFSYNPMGWFRQAPGKGRELVAAISRTG | 412 |
| D03 | GSTYYPDSVEGRFTISRDNAKRMVYLQMNSLRAEDTAVYYCAAGHSWLHFNYW | |
| | GQGTQVTVSS | |
| FAP-FR3- | EVQLVESGGGLVQPGGSLRLSCAASGFIYNYNHMGWFRQAPGKGRELVAAISRTG | 413 |
| A04 | GSTYYPDSVEGRFTISRDNAKRMVYLQMNSLRAEDTAVYYCAVGQEHLHYNYW | |
| | GQGTQVTVSS | |
| FAP-FR3- | EVQLVESGGGLVQPGGSLRLSCAASGRTFSYNPMGWFRQAPGKGRELVAAISRTG | 414 |
| B04 | GSTYYPDSVEGRFTISRDNAKRMVYLQMNSLRAEDTAVYYCAATTYQGRDDGFN | |
| | YWGQGTQVTVSS | |
| FAP-FR3- | EVQLVESGGGLVQPGGSLRLSCAASGSTYSFNTMGWFRQAPGKGRELVASINSSD | 415 |
| E04 | SSTYYPDSVEGRFTISRDNAKRMVYLQMNSLRAEDTAVYYCAAGDYDRHFNYW | |
| | GQGTQVTVSS | |
| FAP-FR3- | EVQLVESGGGLVQPGGSLRLSCAASGSTFSAYSMGWFRQAPGKGRELVAAINSFD | 416 |
| G04 | DITYYPDSVEGRFTISRDNAKRMVYLQMNSLRAEDTAVYYCAAGPLYLHHDYWG | |
| | QGTQVTVSS | |
| FAP-FR3- | EVQLVESGGGLVQPGGSLRLSCAASGSTFNPYFMGWFRQAPGKGRELVAAISRTG | 417 |
| H04 | GSTYYPDSVEGRFTISRDNAKRMVYLQMNSLRAEDTAVYYCAAGWWQMHYNY | |
| | WGQGTQVTVSS | |
| FAP-FR3- | EVQLVESGGGLVQPGGSLRLSCAASGFIFSLYSMGWFRQAPGKGRELVATINALD | 418 |
| D05 | DITYYPDSVEGRFTISRDNAKRMVYLQMNSLRAEDTAVYYCAAGTAPFHYNYWG | |
| | QGTQVTVSS | |
| FAP-FR3- | EVQLVESGGGLVQPGGSLRLSCAASGFIFGYYSMGWFRQAPGKGRELVAAISSFD | 419 |
| E05 | DITYYPDSVEGRFTISRDNAKRMVYLQMNSLRAEDTAVYYCAAHYNRYAPPAAD | |
| | GNTALGTLGYWGQGTQVTVSS | |
| FAP-FR3- | EVQLVESGGGLVQPGGSLRLSCAASGRTFSYNPMGWFRQAPGKGRELVAAISRTG | 420 |
| G05 | GSTYYPDSVEGRFTISRDNAKRMVYLQMNSLRAEDTAVYYCAAGHHYHHYNYW | |
| | GQGTQVTVSS | |
| FAP-FR3- | EVQLVESGGGLVQPGGSLRLSCAASGRTFSYNPMGWFRQAPGKGRELVAAISRTG | 421 |
| H05 | GSTYYPDSVEGRFTISRDNAKRMVYLQMNSLRAEDTAVYYCAASDALAADGVD | |
| | WHYNYWGQGTQVTVSS | |
| FAP-FR3- | EVQLVESGGGLVQPGGSLRLSCAASGRTFSYNPMGWFRQAPGKGRELVAAISRTG | 422 |
| C06 | GSTYYPDSVEGRFTISRDNAKRMVYLQMNSLRAEDTAVYYCAAGDMHHHYNYW | |
| | GQGTQVTVSS | |
| FAP-FR3- | EVQLVESGGGLVQPGGSLRLSCAASGRTFSYNPMGWFRQAPGKGRELVAAISRTG | 423 |
| E06 | GSTYYPDSVEGRFTISRDNAKRMVYLQMNSLRAEDTAVYYCAAGMHGLHYNYW | |
| | GQGTQVTVSS | |
| FAP-FR3- | EVQLVESGGGLVQPGGSLRLSCAASGRIFSSYSMGWFRQAPGKGRELVATINSFSD | 424 |
| F06 | ITYYPDSVEGRFTISRDNAKRMVYLQMNSLRAEDTAVYYCAVGDWDWLFMYWG | |
| | QGTKVTVSS | |
| FAP-FR3- | EVQLVESGGGLVQPGGSLRLSCAASGGISSVYAMGWFRQAPGKGRELVASINSID | 425 |
| G06 | AITYYPDSVEGRFTISRDNAKRMVYLQMNSLRAEDTAVYYCAAGYAPLHFNYWG | |
| | QGTQVTVSS | |
| FAP-FR3- | EVQLVESGGGLVQPGGSLRLSCAASGRTLNVDHMGWFRQAPGKGRELVAAINSID | 426 |
| H06 | SITYYPDSVEGRFTISRDNAKRMVYLQMNSLRAEDTAVYYCAAGYSTFHYNYWG | |
| | QGTQVTVSS | |
| FAP-FR3- | EVQLVESGGGLVQPGGSLRLSCAASGFISSYYYMGWFRQAPGKGRELVAAISRTG | 427 |
| A07 | GSTYYPDSVEGRFTISRDNAKRMVYLQMNSLRAEDTAVYYCAAGDINYHYNYW | |
| | GQGTQVTVSS | |
| FAP-FR3- | EVQLVESGGGLVQPGGSLRLSCAASGYIYSYNFMGWFRQAPGKGRELVASINSFD | 428 |
| B07 | DITYYPDSVEGRFTISRDNAKRMVYLQMNSLRAEDTAVYYCAAGPWNLHFNYW | |
| | GQGTQVTVSS | |
| FAP-FR3- | EVQLVESGGGLVQPGGSLRLSCAASGYTFRIYFMGWFRQAPGKGRELVAAINPYG | 429 |
| D07 | DITNYPDSVEGRFTISRDNAKRMVYLQMNSLRAEDTAVYYCAATFTPGHYNYWG | |
| | QGTQVTVSS | |
| FAP-FR3- | EVQLVESGGGLVQPGGSLRLSCAASGFIYRYYSMGWFRQAPGKGRELVASINAFD | 430 |
| E07 | DITYYPDSVEGRFTISRDNAKRMVYLQMNSLRAEDTAVYYCAAGDYIFHFNYWG | |
| | QGTQVTVSS | |
| FAP-FR3- | EVQLVESGGGLVQPGGSLRLSCAASGSIFDFNHMGWFRQAPGKGRELVASISSFDD | 431 |
| F07 | ITYYPDSVEGRFTISRDNAKRMVYLQMNSLRAEDTAVYYCAAIAHQQKLSYWGQ | |
| | GTQVTVSS | |
| FAP-FR3- | EVQLVESGGGLVQPGGSLRLSCAASGRTFSYNPMGWFRQAPGKGRELVAAISRTG | 432 |
| G07 | GSTYYPDSVEGRFTISRDNAKRMVYLQMNSLRAEDTAVYYCAAGETAYHFNYW | |
| | GQGTQVTVSS | |

What is claimed is:

1. A fibroblast activation protein (FAP) binding polypeptide comprising an immunoglobulin heavy chain variable domain comprising a CDR-H1, CDR-H2, and CDR-H3, wherein:
the CDR-H1 comprises a sequence having at least 90%, 95%, 99%, or 100% sequence identity to a sequence selected from SEQ ID NOs: 1-108;
the CDR-H2 comprises a sequence having at least 90%, 95%, 99%, or 100% sequence identity to a sequence selected from SEQ ID NOs: 109-216; and
the CDR-H3 comprises a sequence having at least 90%, 95%, 99%, or 100% sequence identity to a sequence selected from SEQ ID NOs: 217-324.

2. The FAP binding polypeptide of claim 1, wherein:
1) the CDR-H1 comprises the sequence of SEQ ID NO: 1, the CDR-H2 comprises the sequence of SEQ ID NO: 109, and the CDR-H3 comprises the sequence of SEQ ID NO: 217;
2) the CDR-H1 comprises the sequence of SEQ ID NO: 2, the CDR-H2 comprises the sequence of SEQ ID NO: 110, and the CDR-H3 comprises the sequence of SEQ ID NO: 218;
3) the CDR-H1 comprises the sequence of SEQ ID NO: 3, the CDR-H2 comprises the sequence of SEQ ID NO: 111, and the CDR-H3 comprises the sequence of SEQ ID NO: 219;
4) the CDR-H1 comprises the sequence of SEQ ID NO: 4, the CDR-H2 comprises the sequence of SEQ ID NO: 112, and the CDR-H3 comprises the sequence of SEQ ID NO: 220;
5) the CDR-H1 comprises the sequence of SEQ ID NO: 5, the CDR-H2 comprises the sequence of SEQ ID NO: 113, and the CDR-H3 comprises the sequence of SEQ ID NO: 221;
6) the CDR-H1 comprises the sequence of SEQ ID NO: 6, the CDR-H2 comprises the sequence of SEQ ID NO: 114, and the CDR-H3 comprises the sequence of SEQ ID NO: 222;
7) the CDR-H1 comprises the sequence of SEQ ID NO: 7, the CDR-H2 comprises the sequence of SEQ ID NO: 115, and the CDR-H3 comprises the sequence of SEQ ID NO: 223;
8) the CDR-H1 comprises the sequence of SEQ ID NO: 8, the CDR-H2 comprises the sequence of SEQ ID NO: 116, and the CDR-H3 comprises the sequence of SEQ ID NO: 224;
9) the CDR-H1 comprises the sequence of SEQ ID NO: 9, the CDR-H2 comprises the sequence of SEQ ID NO: 117, and the CDR-H3 comprises the sequence of SEQ ID NO: 225;
10) the CDR-H1 comprises the sequence of SEQ ID NO: 10, the CDR-H2 comprises the sequence of SEQ ID NO: 118, and the CDR-H3 comprises the sequence of SEQ ID NO: 226;
11) the CDR-H1 comprises the sequence of SEQ ID NO: 11, the CDR-H2 comprises the sequence of SEQ ID NO: 119, and the CDR-H3 comprises the sequence of SEQ ID NO: 227;
12) the CDR-H1 comprises the sequence of SEQ ID NO: 12, the CDR-H2 comprises the sequence of SEQ ID NO: 120, and the CDR-H3 comprises the sequence of SEQ ID NO: 228;
13) the CDR-H1 comprises the sequence of SEQ ID NO: 13, the CDR-H2 comprises the sequence of SEQ ID NO: 121, and the CDR-H3 comprises the sequence of SEQ ID NO: 229;
14) the CDR-H1 comprises the sequence of SEQ ID NO: 14, the CDR-H2 comprises the sequence of SEQ ID NO: 122, and the CDR-H3 comprises the sequence of SEQ ID NO: 230;
15) the CDR-H1 comprises the sequence of SEQ ID NO: 15, the CDR-H2 comprises the sequence of SEQ ID NO: 123, and the CDR-H3 comprises the sequence of SEQ ID NO: 231;
16) the CDR-H1 comprises the sequence of SEQ ID NO: 16, the CDR-H2 comprises the sequence of SEQ ID NO: 124, and the CDR-H3 comprises the sequence of SEQ ID NO: 232;
17) the CDR-H1 comprises the sequence of SEQ ID NO: 17, the CDR-H2 comprises the sequence of SEQ ID NO: 125, and the CDR-H3 comprises the sequence of SEQ ID NO: 233;
18) the CDR-H1 comprises the sequence of SEQ ID NO: 18, the CDR-H2 comprises the sequence of SEQ ID NO: 126, and the CDR-H3 comprises the sequence of SEQ ID NO: 234;
19) the CDR-H1 comprises the sequence of SEQ ID NO: 19, the CDR-H2 comprises the sequence of SEQ ID NO: 127, and the CDR-H3 comprises the sequence of SEQ ID NO: 235;
20) the CDR-H1 comprises the sequence of SEQ ID NO: 20, the CDR-H2 comprises the sequence of SEQ ID NO: 128, and the CDR-H3 comprises the sequence of SEQ ID NO: 236;
21) the CDR-H1 comprises the sequence of SEQ ID NO: 21, the CDR-H2 comprises the sequence of SEQ ID NO: 129, and the CDR-H3 comprises the sequence of SEQ ID NO: 237;
22) the CDR-H1 comprises the sequence of SEQ ID NO: 22, the CDR-H2 comprises the sequence of SEQ ID NO: 130, and the CDR-H3 comprises the sequence of SEQ ID NO: 238;
23) the CDR-H1 comprises the sequence of SEQ ID NO: 23, the CDR-H2 comprises the sequence of SEQ ID NO: 131, and the CDR-H3 comprises the sequence of SEQ ID NO: 239;
24) the CDR-H1 comprises the sequence of SEQ ID NO: 24, the CDR-H2 comprises the sequence of SEQ ID NO: 132, and the CDR-H3 comprises the sequence of SEQ ID NO: 240;
25) the CDR-H1 comprises the sequence of SEQ ID NO: 25, the CDR-H2 comprises the sequence of SEQ ID NO: 133, and the CDR-H3 comprises the sequence of SEQ ID NO: 241;
26) the CDR-H1 comprises the sequence of SEQ ID NO: 26, the CDR-H2 comprises the sequence of SEQ ID NO: 134, and the CDR-H3 comprises the sequence of SEQ ID NO: 242;
27) the CDR-H1 comprises the sequence of SEQ ID NO: 27, the CDR-H2 comprises the sequence of SEQ ID NO: 135, and the CDR-H3 comprises the sequence of SEQ ID NO: 243;
28) the CDR-H1 comprises the sequence of SEQ ID NO: 28, the CDR-H2 comprises the sequence of SEQ ID NO: 136, and the CDR-H3 comprises the sequence of SEQ ID NO: 244;
29) the CDR-H1 comprises the sequence of SEQ ID NO: 29, the CDR-H2 comprises the sequence of SEQ ID NO: 137, and the CDR-H3 comprises the sequence of SEQ ID NO: 245;
30) the CDR-H1 comprises the sequence of SEQ ID NO: 30, the CDR-H2 comprises the sequence of SEQ ID NO: 138, and the CDR-H3 comprises the sequence of SEQ ID NO: 246;
31) the CDR-H1 comprises the sequence of SEQ ID NO: 31, the CDR-H2 comprises the sequence of SEQ ID NO: 139, and the CDR-H3 comprises the sequence of SEQ ID NO: 247;
32) the CDR-H1 comprises the sequence of SEQ ID NO: 32, the CDR-H2 comprises the sequence of SEQ ID NO: 140, and the CDR-H3 comprises the sequence of SEQ ID NO: 248;
33) the CDR-H1 comprises the sequence of SEQ ID NO: 33, the CDR-H2 comprises the sequence of SEQ ID NO: 141, and the CDR-H3 comprises the sequence of SEQ ID NO: 249;
34) the CDR-H1 comprises the sequence of SEQ ID NO: 34, the CDR-H2 comprises the sequence of SEQ ID NO: 142, and the CDR-H3 comprises the sequence of SEQ ID NO: 250;
35) the CDR-H1 comprises the sequence of SEQ ID NO: 35, the CDR-H2 comprises the sequence of SEQ ID NO: 143, and the CDR-H3 comprises the sequence of SEQ ID NO: 251;
36) the CDR-H1 comprises the sequence of SEQ ID NO: 36, the CDR-H2 comprises the sequence of SEQ ID NO: 144, and the CDR-H3 comprises the sequence of SEQ ID NO: 252;
37) the CDR-H1 comprises the sequence of SEQ ID NO: 37, the CDR-H2 comprises the sequence of SEQ ID NO: 145, and the CDR-H3 comprises the sequence of SEQ ID NO: 253;
38) the CDR-H1 comprises the sequence of SEQ ID NO: 38, the CDR-H2 comprises the sequence of SEQ ID NO: 146, and the CDR-H3 comprises the sequence of SEQ ID NO: 254;
39) the CDR-H1 comprises the sequence of SEQ ID NO: 39, the CDR-H2 comprises the sequence of SEQ ID NO: 147, and the CDR-H3 comprises the sequence of SEQ ID NO: 255;
40) the CDR-H1 comprises the sequence of SEQ ID NO: 40, the CDR-H2 comprises the sequence of SEQ ID NO: 148, and the CDR-H3 comprises the sequence of SEQ ID NO: 256;
41) the CDR-H1 comprises the sequence of SEQ ID NO: 41, the CDR-H2 comprises the sequence of SEQ ID NO: 149, and the CDR-H3 comprises the sequence of SEQ ID NO: 257;
42) the CDR-H1 comprises the sequence of SEQ ID NO: 42, the CDR-H2 comprises the sequence of SEQ ID NO: 150, and the CDR-H3 comprises the sequence of SEQ ID NO: 258;
43) the CDR-H1 comprises the sequence of SEQ ID NO: 43, the CDR-H2 comprises the sequence of SEQ ID NO: 151, and the CDR-H3 comprises the sequence of SEQ ID NO: 259;
44) the CDR-H1 comprises the sequence of SEQ ID NO: 44, the CDR-H2 comprises the sequence of SEQ ID NO: 152, and the CDR-H3 comprises the sequence of SEQ ID NO: 260;
45) the CDR-H1 comprises the sequence of SEQ ID NO: 45, the CDR-H2 comprises the sequence of SEQ ID NO: 153, and the CDR-H3 comprises the sequence of SEQ ID NO: 261;
46) the CDR-H1 comprises the sequence of SEQ ID NO: 46, the CDR-H2 comprises the sequence of SEQ ID NO: 154, and the CDR-H3 comprises the sequence of SEQ ID NO: 262;
47) the CDR-H1 comprises the sequence of SEQ ID NO: 47, the CDR-H2 comprises the sequence of SEQ ID NO: 155, and the CDR-H3 comprises the sequence of SEQ ID NO: 263;
48) the CDR-H1 comprises the sequence of SEQ ID NO: 48, the CDR-H2 comprises the sequence of SEQ ID NO: 156, and the CDR-H3 comprises the sequence of SEQ ID NO: 264;
49) the CDR-H1 comprises the sequence of SEQ ID NO: 49, the CDR-H2 comprises the sequence of SEQ ID NO: 157, and the CDR-H3 comprises the sequence of SEQ ID NO: 265;
50) the CDR-H1 comprises the sequence of SEQ ID NO: 50, the CDR-H2 comprises the sequence of SEQ ID NO: 158, and the CDR-H3 comprises the sequence of SEQ ID NO: 266;
51) the CDR-H1 comprises the sequence of SEQ ID NO: 51, the CDR-H2 comprises the sequence of SEQ ID NO: 159, and the CDR-H3 comprises the sequence of SEQ ID NO: 267;
52) the CDR-H1 comprises the sequence of SEQ ID NO: 52, the CDR-H2 comprises the sequence of SEQ ID NO: 160, and the CDR-H3 comprises the sequence of SEQ ID NO: 268;
53) the CDR-H1 comprises the sequence of SEQ ID NO: 53, the CDR-H2 comprises the sequence of SEQ ID NO: 161, and the CDR-H3 comprises the sequence of SEQ ID NO: 269;
54) the CDR-H1 comprises the sequence of SEQ ID NO: 54, the CDR-H2 comprises the sequence of SEQ ID NO: 162, and the CDR-H3 comprises the sequence of SEQ ID NO: 270;
55) the CDR-H1 comprises the sequence of SEQ ID NO: 55, the CDR-H2 comprises the sequence of SEQ ID NO: 163, and the CDR-H3 comprises the sequence of SEQ ID NO: 271;
56) the CDR-H1 comprises the sequence of SEQ ID NO: 56, the CDR-H2 comprises the sequence of SEQ ID NO: 164, and the CDR-H3 comprises the sequence of SEQ ID NO: 272;
57) the CDR-H1 comprises the sequence of SEQ ID NO: 57, the CDR-H2 comprises the sequence of SEQ ID NO: 165, and the CDR-H3 comprises the sequence of SEQ ID NO: 273;
58) the CDR-H1 comprises the sequence of SEQ ID NO: 58, the CDR-H2 comprises the sequence of SEQ ID NO: 166, and the CDR-H3 comprises the sequence of SEQ ID NO: 274;
59) the CDR-H1 comprises the sequence of SEQ ID NO: 59, the CDR-H2 comprises the sequence of SEQ ID NO: 167, and the CDR-H3 comprises the sequence of SEQ ID NO: 275;
60) the CDR-H1 comprises the sequence of SEQ ID NO: 60, the CDR-H2 comprises the sequence of SEQ ID NO: 168, and the CDR-H3 comprises the sequence of SEQ ID NO: 276;
61) the CDR-H1 comprises the sequence of SEQ ID NO: 61, the CDR-H2 comprises the sequence of SEQ ID NO: 169, and the CDR-H3 comprises the sequence of SEQ ID NO: 277;
62) the CDR-H1 comprises the sequence of SEQ ID NO: 62, the CDR-H2 comprises the sequence of SEQ ID NO: 170, and the CDR-H3 comprises the sequence of SEQ ID NO: 278;
63) the CDR-H1 comprises the sequence of SEQ ID NO: 63, the CDR-H2 comprises the sequence of SEQ ID NO: 171, and the CDR-H3 comprises the sequence of SEQ ID NO: 279;
64) the CDR-H1 comprises the sequence of SEQ ID NO: 64, the CDR-H2 comprises the sequence of SEQ ID NO: 172, and the CDR-H3 comprises the sequence of SEQ ID NO: 280;
65) the CDR-H1 comprises the sequence of SEQ ID NO: 65, the CDR-H2 comprises the sequence of SEQ ID NO: 173, and the CDR-H3 comprises the sequence of SEQ ID NO: 281;
66) the CDR-H1 comprises the sequence of SEQ ID NO: 66, the CDR-H2 comprises the sequence of SEQ ID NO: 174, and the CDR-H3 comprises the sequence of SEQ ID NO: 282;
67) the CDR-H1 comprises the sequence of SEQ ID NO: 67, the CDR-H2 comprises the sequence of SEQ ID NO: 175, and the CDR-H3 comprises the sequence of SEQ ID NO: 283;
68) the CDR-H1 comprises the sequence of SEQ ID NO: 68, the CDR-H2 comprises the sequence of SEQ ID NO: 176, and the CDR-H3 comprises the sequence of SEQ ID NO: 284;
69) the CDR-H1 comprises the sequence of SEQ ID NO: 69, the CDR-H2 comprises the sequence of SEQ ID NO: 177, and the CDR-H3 comprises the sequence of SEQ ID NO: 285;
70) the CDR-H1 comprises the sequence of SEQ ID NO: 70, the CDR-H2 comprises the sequence of SEQ ID NO: 178, and the CDR-H3 comprises the sequence of SEQ ID NO: 286;
71) the CDR-H1 comprises the sequence of SEQ ID NO: 71, the CDR-H2 comprises the sequence of SEQ ID NO: 179, and the CDR-H3 comprises the sequence of SEQ ID NO: 287;
72) the CDR-H1 comprises the sequence of SEQ ID NO: 72, the CDR-H2 comprises the sequence of SEQ ID NO: 180, and the CDR-H3 comprises the sequence of SEQ ID NO: 288;
73) the CDR-H1 comprises the sequence of SEQ ID NO: 73, the CDR-H2 comprises the sequence of SEQ ID NO: 181, and the CDR-H3 comprises the sequence of SEQ ID NO: 289;
74) the CDR-H1 comprises the sequence of SEQ ID NO: 74, the CDR-H2 comprises the sequence of SEQ ID NO: 182, and the CDR-H3 comprises the sequence of SEQ ID NO: 290;
75) the CDR-H1 comprises the sequence of SEQ ID NO: 75, the CDR-H2 comprises the sequence of SEQ ID NO: 183, and the CDR-H3 comprises the sequence of SEQ ID NO: 291;
76) the CDR-H1 comprises the sequence of SEQ ID NO: 76, the CDR-H2 comprises the sequence of SEQ ID NO: 184, and the CDR-H3 comprises the sequence of SEQ ID NO: 292;
77) the CDR-H1 comprises the sequence of SEQ ID NO: 77, the CDR-H2 comprises the sequence of SEQ ID NO: 185, and the CDR-H3 comprises the sequence of SEQ ID NO: 293;
78) the CDR-H1 comprises the sequence of SEQ ID NO: 78, the CDR-H2 comprises the sequence of SEQ ID NO: 186, and the CDR-H3 comprises the sequence of SEQ ID NO: 294;
79) the CDR-H1 comprises the sequence of SEQ ID NO: 79, the CDR-H2 comprises the sequence of SEQ ID NO: 187, and the CDR-H3 comprises the sequence of SEQ ID NO: 295;
80) the CDR-H1 comprises the sequence of SEQ ID NO: 80, the CDR-H2 comprises the sequence of SEQ ID NO: 188, and the CDR-H3 comprises the sequence of SEQ ID NO: 296;
81) the CDR-H1 comprises the sequence of SEQ ID NO: 81, the CDR-H2 comprises the sequence of SEQ ID NO: 189, and the CDR-H3 comprises the sequence of SEQ ID NO: 297;
82) the CDR-H1 comprises the sequence of SEQ ID NO: 82, the CDR-H2 comprises the sequence of SEQ ID NO: 190, and the CDR-H3 comprises the sequence of SEQ ID NO: 298;
83) the CDR-H1 comprises the sequence of SEQ ID NO: 83, the CDR-H2 comprises the sequence of SEQ ID NO: 191, and the CDR-H3 comprises the sequence of SEQ ID NO: 299;
84) the CDR-H1 comprises the sequence of SEQ ID NO: 84, the CDR-H2 comprises the sequence of SEQ ID NO: 192, and the CDR-H3 comprises the sequence of SEQ ID NO: 300;
85) the CDR-H1 comprises the sequence of SEQ ID NO: 85, the CDR-H2 comprises the sequence of SEQ ID NO: 193, and the CDR-H3 comprises the sequence of SEQ ID NO: 301;
86) the CDR-H1 comprises the sequence of SEQ ID NO: 86, the CDR-H2 comprises the sequence of SEQ ID NO: 194, and the CDR-H3 comprises the sequence of SEQ ID NO: 302;
87) the CDR-H1 comprises the sequence of SEQ ID NO: 87, the CDR-H2 comprises the sequence of SEQ ID NO: 195, and the CDR-H3 comprises the sequence of SEQ ID NO: 303;
88) the CDR-H1 comprises the sequence of SEQ ID NO: 88, the CDR-H2 comprises the sequence of SEQ ID NO: 196, and the CDR-H3 comprises the sequence of SEQ ID NO: 304;
89) the CDR-H1 comprises the sequence of SEQ ID NO: 89, the CDR-H2 comprises the sequence of SEQ ID NO: 197, and the CDR-H3 comprises the sequence of SEQ ID NO: 305;
90) the CDR-H1 comprises the sequence of SEQ ID NO: 90, the CDR-H2 comprises the sequence of SEQ ID NO: 198, and the CDR-H3 comprises the sequence of SEQ ID NO: 306;
91) the CDR-H1 comprises the sequence of SEQ ID NO: 91, the CDR-H2 comprises the sequence of SEQ ID NO: 199, and the CDR-H3 comprises the sequence of SEQ ID NO: 307;
92) the CDR-H1 comprises the sequence of SEQ ID NO: 92, the CDR-H2 comprises the sequence of SEQ ID NO: 200, and the CDR-H3 comprises the sequence of SEQ ID NO: 308;
93) the CDR-H1 comprises the sequence of SEQ ID NO: 93, the CDR-H2 comprises the sequence of SEQ ID NO: 201, and the CDR-H3 comprises the sequence of SEQ ID NO: 309;
94) the CDR-H1 comprises the sequence of SEQ ID NO: 94, the CDR-H2 comprises the sequence of SEQ ID NO: 202, and the CDR-H3 comprises the sequence of SEQ ID NO: 310;
95) the CDR-H1 comprises the sequence of SEQ ID NO: 95, the CDR-H2 comprises the sequence of SEQ ID NO: 203, and the CDR-H3 comprises the sequence of SEQ ID NO: 311;
96) the CDR-H1 comprises the sequence of SEQ ID NO: 96, the CDR-H2 comprises the sequence of SEQ ID NO: 204, and the CDR-H3 comprises the sequence of SEQ ID NO: 312;
97) the CDR-H1 comprises the sequence of SEQ ID NO: 97, the CDR-H2 comprises the sequence of SEQ ID NO: 205, and the CDR-H3 comprises the sequence of SEQ ID NO: 313;
98) the CDR-H1 comprises the sequence of SEQ ID NO: 98, the CDR-H2 comprises the sequence of SEQ ID NO: 206, and the CDR-H3 comprises the sequence of SEQ ID NO: 314;
99) the CDR-H1 comprises the sequence of SEQ ID NO: 99, the CDR-H2 comprises the sequence of SEQ ID NO: 207, and the CDR-H3 comprises the sequence of SEQ ID NO: 315;
100) the CDR-H1 comprises the sequence of SEQ ID NO: 100, the CDR-H2 comprises the sequence of SEQ ID NO: 208, and the CDR-H3 comprises the sequence of SEQ ID NO: 316;
101) the CDR-H1 comprises the sequence of SEQ ID NO: 101, the CDR-H2 comprises the sequence of SEQ ID NO: 209, and the CDR-H3 comprises the sequence of SEQ ID NO: 317;
102) the CDR-H1 comprises the sequence of SEQ ID NO: 102, the CDR-H2 comprises the sequence of SEQ ID NO: 210, and the CDR-H3 comprises the sequence of SEQ ID NO: 318;
103) the CDR-H1 comprises the sequence of SEQ ID NO: 103, the CDR-H2 comprises the sequence of SEQ ID NO: 211, and the CDR-H3 comprises the sequence of SEQ ID NO: 319;
104) the CDR-H1 comprises the sequence of SEQ ID NO: 104, the CDR-H2 comprises the sequence of SEQ ID NO: 212, and the CDR-H3 comprises the sequence of SEQ ID NO: 320;
105) the CDR-H1 comprises the sequence of SEQ ID NO: 105, the CDR-H2 comprises the sequence of SEQ ID NO: 213, and the CDR-H3 comprises the sequence of SEQ ID NO: 321;
106) the CDR-H1 comprises the sequence of SEQ ID NO: 106, the CDR-H2 comprises the sequence of SEQ ID NO: 214, and the CDR-H3 comprises the sequence of SEQ ID NO: 322;
107) the CDR-H1 comprises the sequence of SEQ ID NO: 107, the CDR-H2 comprises the sequence of SEQ ID NO: 215, and the CDR-H3 comprises the sequence of SEQ ID NO: 323; or
108) the CDR-H1 comprises the sequence of SEQ ID NO: 108, the CDR-H2 comprises the sequence of SEQ ID NO: 216, and the CDR-H3 comprises the sequence of SEQ ID NO: 324.

3. The FAP binding polypeptide of claim 1, wherein the heavy chain variable domain comprises an amino acid sequence having at least 90%, 95%, 99%, or 100% sequence identity to any sequence selected from SEQ ID NOs: 325-432.

4. The FAP binding polypeptide of claim 1, wherein the FAP binding polypeptide is humanized.

5. The FAP binding polypeptide of claim 1, wherein the FAP binding polypeptide is a single domain antibody (sdAb).

6. A FAP binding polypeptide comprising an immunoglobulin heavy chain variable domain comprising a CDR-H1, wherein the CDR-H1 comprises a sequence having at least 90%, 95%, 99%, or 100% sequence identity to a sequence selected from SEQ ID NOs: 1-108.

7. The FAP binding polypeptide of claim 6, wherein the CDR-H1 comprises a sequence having at least 90%, 95%, 99%, or 100% sequence identity to a sequence selected from SEQ ID NO: 4.

8. The FAP binding polypeptide of claim 6, wherein the immunoglobulin heavy chain variable domain further comprises a CDR-H2, wherein the CDR-H2 comprises a sequence having at least 90%, 95%, 99%, or 100% sequence identity to a sequence selected from SEQ ID NOs: 109-216.

9. The FAP binding polypeptide of claim 6, wherein the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324.

10. The FAP binding polypeptide of claim 6, wherein the immunoglobulin heavy chain variable domain comprises an amino acid sequence having at least 90%, 95%, 99%, or 100% sequence identity to any sequence selected from SEQ ID NOs: 325-432.

11. A FAP binding polypeptide comprising an immunoglobulin heavy chain variable domain comprising a CDR-H2, wherein the CDR-H2 comprises a sequence having at least 90%, 95%, 99%, or 100% sequence identity to a sequence selected from SEQ ID NOs: 109-216.

12. The FAP binding polypeptide of claim 11, wherein the CDR-H2 comprises a sequence having at least 90%, 95%, 99%, or 100% sequence identity to a sequence selected from SEQ ID NO: 110.

13. The FAP binding polypeptide of claim 11, wherein the immunoglobulin heavy chain variable domain further comprises a CDR-H1, wherein the CDR-H1 comprises a sequence having at least 90%, 95%, 99%, or 100% sequence identity to a sequence selected from SEQ ID NOs: 1-108.

14. The FAP binding polypeptide of claim 11, wherein the immunoglobulin heavy chain variable domain further comprises a CDR-H3, wherein the CDR-H3 comprises a sequence having at least 90%, 95%, 99%, or 100% sequence identity to a sequence selected from SEQ ID NOs: 217-324.

15. The FAP binding polypeptide of claim 11, wherein the immunoglobulin heavy chain variable domain comprises an amino acid sequence having at least 90%, 95%, 99%, or 100% sequence identity to any sequence selected from SEQ ID NOs: 325-432.

16. A chimeric antigen receptor (CAR) comprising the FAP binding polypeptide of claim 1.

17. A chimeric antigen receptor (CAR) cell comprising the CAR of claim 16.

18. The CAR cell of claim 17, wherein the CAR cell is a CAR T cell.

19. The CAR cell of claim 17, wherein the CAR cell comprises at least two binding polypeptides and the CAR cell is a multivalent CAR cell.

20. The CAR cell of claim 17, wherein the CAR cell is derived from a subject or from a cell line.

21. The CAR cell of claim 20, wherein the subject has a cancer.

22. The CAR cell of claim 21, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

23. A nucleic acid that encodes for a polypeptide comprising a sequence having at least 90% sequence identity to the FAP binding polypeptide of claim 1.

24. A method of treating a cancer in a subject in need thereof, comprising administering the CAR cell of claim 17.

25. The method of claim 24, wherein the chimeric antigen receptor cell is autologous or allogeneic to the subject.

26. The method of claim 24, wherein the subject is a mammal.

27. The method of claim 24, wherein the subject is a human.

28. The method of claim 24, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

29. The method of claim 24, wherein the chimeric antigen receptor cell is administered parenterally.


SEQUENCE LISTING (432 sequences)

SEQ ID NO: 1 - protein, 9 aa, Artificial Sequence
  FAP-FR2-A01 HCDR1
GSTSNNYFM

SEQ ID NO: 2 - protein, 9 aa, Artificial Sequence
  FAP-FR2-B01 HCDR1
GFTSGVYFM

SEQ ID NO: 3 - protein, 9 aa, Artificial Sequence
  FAP-FR2-C01 HCDR1
GFTFGFNYM

SEQ ID NO: 4 - protein, 9 aa, Artificial Sequence
  FAP-FR2-E01 HCDR1
GRTFSYNPM

SEQ ID NO: 5 - protein, 9 aa, Artificial Sequence
  FAP-FR2-F01 HCDR1
GRTFSYNPM

SEQ ID NO: 6 - protein, 9 aa, Artificial Sequence
  FAP-FR2-G01 HCDR1
GSIFSHYSM

SEQ ID NO: 7 - protein, 9 aa, Artificial Sequence
  FAP-FR2-H01 HCDR1
GYILRDDPM

SEQ ID NO: 8 - protein, 9 aa, Artificial Sequence
  FAP-FR2-B02 HCDR1
GSTFSYYFM

SEQ ID NO: 9 - protein, 9 aa, Artificial Sequence
  FAP-FR2-C02 HCDR1
GRTFSYNPM

SEQ ID NO: 10 - protein, 9 aa, Artificial Sequence
  FAP-FR2-D02 HCDR1
GFIYSNYYM

SEQ ID NO: 11 - protein, 9 aa, Artificial Sequence
  FAP-FR2-E02 HCDR1
GFIFSAYYM

SEQ ID NO: 12 - protein, 9 aa, Artificial Sequence
  FAP-FR2-H02 HCDR1
GSTYDVDYM

SEQ ID NO: 13 - protein, 9 aa, Artificial Sequence
  FAP-FR2-B03 HCDR1
GSIYSFYFM

SEQ ID NO: 14 - protein, 9 aa, Artificial Sequence
  FAP-FR2-C03 HCDR1
GRTFSYNPM

SEQ ID NO: 15 - protein, 9 aa, Artificial Sequence
  FAP-FR2-D03 HCDR1
GRIYRNYSM

SEQ ID NO: 16 - protein, 9 aa, Artificial Sequence
  FAP-FR2-F03 HCDR1
GFIFSFYFM

SEQ ID NO: 17 - protein, 9 aa, Artificial Sequence
  FAP-FR2-H03 HCDR1
GYIFSVNFM

SEQ ID NO: 18 - protein, 9 aa, Artificial Sequence
  FAP-FR2-B04 HCDR1
GSIFDYYYM

SEQ ID NO: 19 - protein, 9 aa, Artificial Sequence
  FAP-FR2-D04 HCDR1
GSIYSNYYM

SEQ ID NO: 20 - protein, 9 aa, Artificial Sequence
  FAP-FR2-E04 HCDR1
GFIFNIYSM

SEQ ID NO: 21 - protein, 9 aa, Artificial Sequence
  FAP-FR2-G04 HCDR1
GSIYDLYTM

SEQ ID NO: 22 - protein, 9 aa, Artificial Sequence
  FAP-FR2-A05 HCDR1
GRIYDLYNM

SEQ ID NO: 23 - protein, 9 aa, Artificial Sequence
  FAP-FR2-C05 HCDR1
GRTFSYNPM

SEQ ID NO: 24 - protein, 9 aa, Artificial Sequence
  FAP-FR2-D05 HCDR1
GFTYGFNYM

SEQ ID NO: 25 - protein, 9 aa, Artificial Sequence
  FAP-FR2-E05 HCDR1
GRTFSYNPM

SEQ ID NO: 26 - protein, 9 aa, Artificial Sequence
  FAP-FR2-F05 HCDR1
GSIYRFDSM

SEQ ID NO: 27 - protein, 9 aa, Artificial Sequence
  FAP-FR2-G05 HCDR1
GFISSAYSM

SEQ ID NO: 28 - protein, 9 aa, Artificial Sequence
  FAP-FR2-H05 HCDR1
GRTFSYNPM

SEQ ID NO: 29 - protein, 9 aa, Artificial Sequence
  FAP-FR2-A06 HCDR1
GSIFSSYIM

SEQ ID NO: 30 - protein, 9 aa, Artificial Sequence
  FAP-FR2-B06 HCDR1
GFTLNIYHM

SEQ ID NO: 31 - protein, 9 aa, Artificial Sequence
  FAP-FR2-C06 HCDR1
GGTFSFYFM

SEQ ID NO: 32 - protein, 9 aa, Artificial Sequence
  FAP-FR2-D06 HCDR1
GRIYDANFM

SEQ ID NO: 33 - protein, 9 aa, Artificial Sequence
  FAP-FR2-E06 HCDR1
GRTFSYNPM

SEQ ID NO: 34 - protein, 9 aa, Artificial Sequence
  FAP-FR2-F06 HCDR1
GRTFSYNPM

SEQ ID NO: 35 - protein, 9 aa, Artificial Sequence
  FAP-FR2-A07 HCDR1
GFIFDYYFM

SEQ ID NO: 36 - protein, 9 aa, Artificial Sequence
  FAP-FR2-B07 HCDR1
GRTFSYNPM

SEQ ID NO: 37 - protein, 9 aa, Artificial Sequence
  FAP-FR2-C07 HCDR1
GFTFSFYFM

SEQ ID NO: 38 - protein, 9 aa, Artificial Sequence
  FAP-FR2-D07 HCDR1
GRIFSSYSM

SEQ ID NO: 39 - protein, 9 aa, Artificial Sequence
  FAP-FR2-E07 HCDR1
GSTFGYDNM

SEQ ID NO: 40 - protein, 9 aa, Artificial Sequence
  FAP-FR2-F07 HCDR1
GRTFSYNPM

SEQ ID NO: 41 - protein, 9 aa, Artificial Sequence
  FAP-FR2-G07 HCDR1
GGISDYYSM

SEQ ID NO: 42 - protein, 9 aa, Artificial Sequence
  FAP-FR2-H07 HCDR1
GYIFSFYFM

SEQ ID NO: 43 - protein, 9 aa, Artificial Sequence
  FAP-FR2-A08 HCDR1
GFIFGIYFM

SEQ ID NO: 44 - protein, 9 aa, Artificial Sequence
  FAP-FR2-C08 HCDR1
GSIYGYDYM

SEQ ID NO: 45 - protein, 9 aa, Artificial Sequence
  FAP-FR2-D08 HCDR1
GSILGSYIM

SEQ ID NO: 46 - protein, 9 aa, Artificial Sequence
  FAP-FR2-E08 HCDR1
GFIYSFYVM

SEQ ID NO: 47 - protein, 9 aa, Artificial Sequence
  FAP-FR2-G08 HCDR1
GRTFSYNPM

SEQ ID NO: 48 - protein, 9 aa, Artificial Sequence
  FAP-FR2-A09 HCDR1
GFTFSFYSM

SEQ ID NO: 49 - protein, 9 aa, Artificial Sequence
  FAP-FR2-B09 HCDR1
GGTFGVNPM

SEQ ID NO: 50 - protein, 9 aa, Artificial Sequence
  FAP-FR2-C09 HCDR1
GRTFSYNPM

SEQ ID NO: 51 - protein, 9 aa, Artificial Sequence
  FAP-FR2-D09 HCDR1
GRTFSYNPM

SEQ ID NO: 52 - protein, 9 aa, Artificial Sequence
  FAP-FR2-E09 HCDR1
GFISGHYYM

SEQ ID NO: 53 - protein, 9 aa, Artificial Sequence
  FAP-FR2-F09 HCDR1
GGILRIYFM

SEQ ID NO: 54 - protein, 9 aa, Artificial Sequence
  FAP-FR2-G09 HCDR1
GRTFSYNPM

SEQ ID NO: 55 - protein, 9 aa, Artificial Sequence
  FAP-FR2-H09 HCDR1
GFIFNVYYM

SEQ ID NO: 56 - protein, 9 aa, Artificial Sequence
  FAP-FR2-B10 HCDR1
GSIYSFYFM

SEQ ID NO: 57 - protein, 9 aa, Artificial Sequence
  FAP-FR2-C10 HCDR1
GRTFSYNPM

SEQ ID NO: 58 - protein, 9 aa, Artificial Sequence
  FAP-FR2-D10 HCDR1
GSIFGAYYM

SEQ ID NO: 59 - protein, 9 aa, Artificial Sequence
  FAP-FR2-E10 HCDR1
GSTFRFYSM

SEQ ID NO: 60 - protein, 9 aa, Artificial Sequence
  FAP-FR2-F10 HCDR1
GFILGSDTM

SEQ ID NO: 61 - protein, 9 aa, Artificial Sequence
  FAP-FR2-G10 HCDR1
GFIFSVYSM

SEQ ID NO: 62 - protein, 9 aa, Artificial Sequence
  FAP-FR2-H10 HCDR1
GRTFNFYAM

SEQ ID NO: 63 - protein, 9 aa, Artificial Sequence
  FAP-FR2-A11 HCDR1
GRTLSSYTM

SEQ ID NO: 64 - protein, 9 aa, Artificial Sequence
  FAP-FR2-C11 HCDR1
GRTFSYNPM

SEQ ID NO: 65 - protein, 9 aa, Artificial Sequence
  FAP-FR2-D11 HCDR1
GYISSSYSM

SEQ ID NO: 66 - protein, 9 aa, Artificial Sequence
  FAP-FR2-F11 HCDR1
GRTFSYNPM

SEQ ID NO: 67 - protein, 9 aa, Artificial Sequence
  FAP-FR2-G11 HCDR1
GSIYSHNFM

SEQ ID NO: 68 - protein, 9 aa, Artificial Sequence
  FAP-FR2-H11 HCDR1
GFIFSFYSM

SEQ ID NO: 69 - protein, 9 aa, Artificial Sequence
  FAP-FR2-B12 HCDR1
GFIFDFYFM

SEQ ID NO: 70 - protein, 9 aa, Artificial Sequence
  FAP-FR2-C12 HCDR1
GFIYSSNNM

SEQ ID NO: 71 - protein, 9 aa, Artificial Sequence
  FAP-FR2-D12 HCDR1
GRILRHYHM

SEQ ID NO: 72 - protein, 9 aa, Artificial Sequence
  FAP-FR2-F12 HCDR1
GFIFGSYFM

SEQ ID NO: 73 - protein, 9 aa, Artificial Sequence
  FAP-FR2-G12 HCDR1
GYIFSSYAM

SEQ ID NO: 74 - protein, 9 aa, Artificial Sequence
  FAP-FR2-H12 HCDR1
GFTFGAYHM

SEQ ID NO: 75 - protein, 9 aa, Artificial Sequence
  FAP-FR3-A01 HCDR1
GRTFGLDFM

SEQ ID NO: 76 - protein, 9 aa, Artificial Sequence
  FAP-FR3-B01 HCDR1
GYILSFYYM

SEQ ID NO: 77 - protein, 9 aa, Artificial Sequence
  FAP-FR3-E01 HCDR1
GFISSLYSM

SEQ ID NO: 78 - protein, 9 aa, Artificial Sequence
  FAP-FR3-F01 HCDR1
GFIFNYYYM

SEQ ID NO: 79 - protein, 9 aa, Artificial Sequence
  FAP-FR3-H01 HCDR1
GSTSGAYNM

SEQ ID NO: 80 - protein, 9 aa, Artificial Sequence
  FAP-FR3-A02 HCDR1
GFIYNFNSM

SEQ ID NO: 81 - protein, 9 aa, Artificial Sequence
  FAP-FR3-C02 HCDR1
GSTSRDYSM

SEQ ID NO: 82 - protein, 9 aa, Artificial Sequence
  FAP-FR3-D02 HCDR1
GSTYNVYPM

SEQ ID NO: 83 - protein, 9 aa, Artificial Sequence
  FAP-FR3-F02 HCDR1
GRTFSYNPM

SEQ ID NO: 84 - protein, 9 aa, Artificial Sequence
  FAP-FR3-G02 HCDR1
GSTFNYYDM

SEQ ID NO: 85 - protein, 9 aa, Artificial Sequence
  FAP-FR3-A03 HCDR1
GFISGVYFM

SEQ ID NO: 86 - protein, 9 aa, Artificial Sequence
  FAP-FR3-B03 HCDR1
GFILGANPM

SEQ ID NO: 87 - protein, 9 aa, Artificial Sequence
  FAP-FR3-C03 HCDR1
GFIYGYNHM

SEQ ID NO: 88 - protein, 9 aa, Artificial Sequence
  FAP-FR3-D03 HCDR1
GRTFSYNPM

SEQ ID NO: 89 - protein, 9 aa, Artificial Sequence
  FAP-FR3-A04 HCDR1
GFIYNYNHM

SEQ ID NO: 90 - protein, 9 aa, Artificial Sequence
  FAP-FR3-B04 HCDR1
GRTFSYNPM

SEQ ID NO: 91 - protein, 9 aa, Artificial Sequence
  FAP-FR3-E04 HCDR1
GSTYSFNTM

SEQ ID NO: 92 - protein, 9 aa, Artificial Sequence
  FAP-FR3-G04 HCDR1
GSTFSAYSM

SEQ ID NO: 93 - protein, 9 aa, Artificial Sequence
  FAP-FR3-H04 HCDR1
GSTFNPYFM

SEQ ID NO: 94 - protein, 9 aa, Artificial Sequence
  FAP-FR3-D05 HCDR1
GFIFSLYSM

SEQ ID NO: 95 - protein, 9 aa, Artificial Sequence
  FAP-FR3-E05 HCDR1
GFIFGYYSM

SEQ ID NO: 96 - protein, 9 aa, Artificial Sequence
  FAP-FR3-G05 HCDR1
GRTFSYNPM

SEQ ID NO: 97 - protein, 9 aa, Artificial Sequence
  FAP-FR3-H05 HCDR1
GRTFSYNPM

SEQ ID NO: 98 - protein, 9 aa, Artificial Sequence
  FAP-FR3-C06 HCDR1
GRTFSYNPM

SEQ ID NO: 99 - protein, 9 aa, Artificial Sequence
  FAP-FR3-E06 HCDR1
GRTFSYNPM

SEQ ID NO: 100 - protein, 9 aa, Artificial Sequence
  FAP-FR3-F06 HCDR1
GRIFSSYSM

SEQ ID NO: 101 - protein, 9 aa, Artificial Sequence
  FAP-FR3-G06 HCDR1
GGISSVYAM

SEQ ID NO: 102 - protein, 9 aa, Artificial Sequence
  FAP-FR3-H06 HCDR1
GRTLNVDHM

SEQ ID NO: 103 - protein, 9 aa, Artificial Sequence
  FAP-FR3-A07 HCDR1
GFISSYYYM

SEQ ID NO: 104 - protein, 9 aa, Artificial Sequence
  FAP-FR3-B07 HCDR1
GYIYSYNFM

SEQ ID NO: 105 - protein, 9 aa, Artificial Sequence
  FAP-FR3-D07 HCDR1
GYTFRIYFM

SEQ ID NO: 106 - protein, 9 aa, Artificial Sequence
  FAP-FR3-E07 HCDR1
GFIYRYYSM

SEQ ID NO: 107 - protein, 9 aa, Artificial Sequence
  FAP-FR3-F07 HCDR1
GSIFDFNHM

SEQ ID NO: 108 - protein, 9 aa, Artificial Sequence
  FAP-FR3-G07 HCDR1
GRTFSYNPM

SEQ ID NO: 109 - protein, 10 aa, Artificial Sequence
  FAP-FR2-A01 HCDR2
AINPLDSNTY

SEQ ID NO: 110 - protein, 10 aa, Artificial Sequence
  FAP-FR2-B01 HCDR2
AISRTGGSTY

SEQ ID NO: 111 - protein, 10 aa, Artificial Sequence
  FAP-FR2-C01 HCDR2
AISRISDSTY

SEQ ID NO: 112 - protein, 10 aa, Artificial Sequence
  FAP-FR2-E01 HCDR2
AISRTGGSTY

SEQ ID NO: 113 - protein, 10 aa, Artificial Sequence
  FAP-FR2-F01 HCDR2
AISRTGGSTY

SEQ ID NO: 114 - protein, 10 aa, Artificial Sequence
  FAP-FR2-G01 HCDR2
AISRTGGSTY

SEQ ID NO: 115 - protein, 10 aa, Artificial Sequence
  FAP-FR2-H01 HCDR2
AISRTGGSTY

SEQ ID NO: 116 - protein, 10 aa, Artificial Sequence
  FAP-FR2-B02 HCDR2
AISRTGGSTY

SEQ ID NO: 117 - protein, 10 aa, Artificial Sequence
  FAP-FR2-C02 HCDR2
AISRTGGSTY

SEQ ID NO: 118 - protein, 10 aa, Artificial Sequence
  FAP-FR2-D02 HCDR2
AISRTGGSTY

SEQ ID NO: 119 - protein, 10 aa, Artificial Sequence
  FAP-FR2-E02 HCDR2
TINSVDDITY

SEQ ID NO: 120 - protein, 10 aa, Artificial Sequence
  FAP-FR2-H02 HCDR2
TINSYGGITY

SEQ ID NO: 121 - protein, 10 aa, Artificial Sequence
  FAP-FR2-B03 HCDR2
AISRTGGSTY

SEQ ID NO: 122 - protein, 10 aa, Artificial Sequence
  FAP-FR2-C03 HCDR2
AISRTGGSTY

SEQ ID NO: 123 - protein, 10 aa, Artificial Sequence
  FAP-FR2-D03 HCDR2
AISRTGGSTY

SEQ ID NO: 124 - protein, 10 aa, Artificial Sequence
  FAP-FR2-F03 HCDR2
AISRTGGSTY

SEQ ID NO: 125 - protein, 10 aa, Artificial Sequence
  FAP-FR2-H03 HCDR2
TINWFDDITY

SEQ ID NO: 126 - protein, 10 aa, Artificial Sequence
  FAP-FR2-B04 HCDR2
AISRTGGSTY

SEQ ID NO: 127 - protein, 10 aa, Artificial Sequence
  FAP-FR2-D04 HCDR2
AISRTGGSTY

SEQ ID NO: 128 - protein, 10 aa, Artificial Sequence
  FAP-FR2-E04 HCDR2
SINWSDSSTY

SEQ ID NO: 129 - protein, 10 aa, Artificial Sequence
  FAP-FR2-G04 HCDR2
AISRTGGSTY

SEQ ID NO: 130 - protein, 10 aa, Artificial Sequence
  FAP-FR2-A05 HCDR2
AISRTGGSTY

SEQ ID NO: 131 - protein, 10 aa, Artificial Sequence
  FAP-FR2-C05 HCDR2
AISRTGGSTY

SEQ ID NO: 132 - protein, 10 aa, Artificial Sequence
  FAP-FR2-D05 HCDR2
AISRTGGSTY

SEQ ID NO: 133 - protein, 10 aa, Artificial Sequence
  FAP-FR2-E05 HCDR2
AISRTGGSTY

SEQ ID NO: 134 - protein, 10 aa, Artificial Sequence
  FAP-FR2-F05 HCDR2
TITSLDSITY

SEQ ID NO: 135 - protein, 10 aa, Artificial Sequence
  FAP-FR2-G05 HCDR2
AISRTGGSTY

SEQ ID NO: 136 - protein, 10 aa, Artificial Sequence
  FAP-FR2-H05 HCDR2
AISRTGGSTY

SEQ ID NO: 137 - protein, 10 aa, Artificial Sequence
  FAP-FR2-A06 HCDR2
SINSYDSTTY

SEQ ID NO: 138 - protein, 10 aa, Artificial Sequence
  FAP-FR2-B06 HCDR2
AINSFSDITY

SEQ ID NO: 139 - protein, 10 aa, Artificial Sequence
  FAP-FR2-C06 HCDR2
TINSFDDITY

SEQ ID NO: 140 - protein, 10 aa, Artificial Sequence
  FAP-FR2-D06 HCDR2
TINTYSDITY

SEQ ID NO: 141 - protein, 10 aa, Artificial Sequence
  FAP-FR2-E06 HCDR2
AISRTGGSTY

SEQ ID NO: 142 - protein, 10 aa, Artificial Sequence
  FAP-FR2-F06 HCDR2
AISRTGGSTY

SEQ ID NO: 143 - protein, 10 aa, Artificial Sequence
  FAP-FR2-A07 HCDR2
TINSFSSSTY

SEQ ID NO: 144 - protein, 9 aa, Artificial Sequence
  FAP-FR2-B07 HCDR2
TISFSGITY

SEQ ID NO: 145 - protein, 10 aa, Artificial Sequence
  FAP-FR2-C07 HCDR2
SINAYSDSTY

SEQ ID NO: 146 - protein, 10 aa, Artificial Sequence
  FAP-FR2-D07 HCDR2
TISSFSDITY

SEQ ID NO: 147 - protein, 10 aa, Artificial Sequence
  FAP-FR2-E07 HCDR2
TINSFGDITN

SEQ ID NO: 148 - protein, 10 aa, Artificial Sequence
  FAP-FR2-F07 HCDR2
AISRTGGSTY

SEQ ID NO: 149 - protein, 10 aa, Artificial Sequence
  FAP-FR2-G07 HCDR2
AISRTGGSTY

SEQ ID NO: 150 - protein, 10 aa, Artificial Sequence
  FAP-FR2-H07 HCDR2
AISRTGGSTY

SEQ ID NO: 151 - protein, 10 aa, Artificial Sequence
  FAP-FR2-A08 HCDR2
AISRTGGSTY

SEQ ID NO: 152 - protein, 10 aa, Artificial Sequence
  FAP-FR2-C08 HCDR2
AISRTGGSTY

SEQ ID NO: 153 - protein, 10 aa, Artificial Sequence
  FAP-FR2-D08 HCDR2
TINSNDSITY

SEQ ID NO: 154 - protein, 10 aa, Artificial Sequence
  FAP-FR2-E08 HCDR2
TINSFDDTTY

SEQ ID NO: 155 - protein, 10 aa, Artificial Sequence
  FAP-FR2-G08 HCDR2
AISRTGGSTY

SEQ ID NO: 156 - protein, 10 aa, Artificial Sequence
  FAP-FR2-A09 HCDR2
SINAIDSITY

SEQ ID NO: 157 - protein, 10 aa, Artificial Sequence
  FAP-FR2-B09 HCDR2
TITSTDDITY

SEQ ID NO: 158 - protein, 10 aa, Artificial Sequence
  FAP-FR2-C09 HCDR2
AISRTGGSTY

SEQ ID NO: 159 - protein, 10 aa, Artificial Sequence
  FAP-FR2-D09 HCDR2
AISRTGGSTY

SEQ ID NO: 160 - protein, 10 aa, Artificial Sequence
  FAP-FR2-E09 HCDR2
AISRTGGSTY

SEQ ID NO: 161 - protein, 10 aa, Artificial Sequence
  FAP-FR2-F09 HCDR2
AISRTGGSTY

SEQ ID NO: 162 - protein, 10 aa, Artificial Sequence
  FAP-FR2-G09 HCDR2
AISRTGGSTY

SEQ ID NO: 163 - protein, 10 aa, Artificial Sequence
  FAP-FR2-H09 HCDR2
TISWYSDITY

SEQ ID NO: 164 - protein, 10 aa, Artificial Sequence
  FAP-FR2-B10 HCDR2
TINSFDSITY

SEQ ID NO: 165 - protein, 10 aa, Artificial Sequence
  FAP-FR2-C10 HCDR2
AISRTGGSTY

SEQ ID NO: 166 - protein, 10 aa, Artificial Sequence
  FAP-FR2-D10 HCDR2
AINPDSDITY

SEQ ID NO: 167 - protein, 10 aa, Artificial Sequence
  FAP-FR2-E10 HCDR2
TISSFDDITY

SEQ ID NO: 168 - protein, 10 aa, Artificial Sequence
  FAP-FR2-F10 HCDR2
SINSFDSITY

SEQ ID NO: 169 - protein, 10 aa, Artificial Sequence
  FAP-FR2-G10 HCDR2
AINTVDSITY

SEQ ID NO: 170 - protein, 10 aa, Artificial Sequence
  FAP-FR2-H10 HCDR2
AISRTGGSTY

SEQ ID NO: 171 - protein, 10 aa, Artificial Sequence
  FAP-FR2-A11 HCDR2
SISWVDSITY

SEQ ID NO: 172 - protein, 10 aa, Artificial Sequence
  FAP-FR2-C11 HCDR2
AISRTGGSTY

SEQ ID NO: 173 - protein, 10 aa, Artificial Sequence
  FAP-FR2-D11 HCDR2
AISSFSDITY

SEQ ID NO: 174 - protein, 10 aa, Artificial Sequence
  FAP-FR2-F11 HCDR2
AISRTGGSTY

SEQ ID NO: 175 - protein, 10 aa, Artificial Sequence
  FAP-FR2-G11 HCDR2
AISRTGGSTY

SEQ ID NO: 176 - protein, 10 aa, Artificial Sequence
  FAP-FR2-H11 HCDR2
TINTFSDITY

SEQ ID NO: 177 - protein, 10 aa, Artificial Sequence
  FAP-FR2-B12 HCDR2
TINSISDITY

SEQ ID NO: 178 - protein, 10 aa, Artificial Sequence
  FAP-FR2-C12 HCDR2
SINSFDDSTY

SEQ ID NO: 179 - protein, 10 aa, Artificial Sequence
  FAP-FR2-D12 HCDR2
TINTLDSITY

SEQ ID NO: 180 - protein, 10 aa, Artificial Sequence
  FAP-FR2-F12 HCDR2
AISRTGGSTY

SEQ ID NO: 181 - protein, 10 aa, Artificial Sequence
  FAP-FR2-G12 HCDR2
AISRTGGSTY

SEQ ID NO: 182 - protein, 10 aa, Artificial Sequence
  FAP-FR2-H12 HCDR2
TINWFGGITY

SEQ ID NO: 183 - protein, 10 aa, Artificial Sequence
  FAP-FR3-A01 HCDR2
SINSFDDITN

SEQ ID NO: 184 - protein, 10 aa, Artificial Sequence
  FAP-FR3-B01 HCDR2
TINSFSDITY

SEQ ID NO: 185 - protein, 10 aa, Artificial Sequence
  FAP-FR3-E01 HCDR2
TITGSSSITY

SEQ ID NO: 186 - protein, 10 aa, Artificial Sequence
  FAP-FR3-F01 HCDR2
TISAFDDSTY

SEQ ID NO: 187 - protein, 10 aa, Artificial Sequence
  FAP-FR3-H01 HCDR2
TINWFDDITY

SEQ ID NO: 188 - protein, 10 aa, Artificial Sequence
  FAP-FR3-A02 HCDR2
AINRFDDITY

SEQ ID NO: 189 - protein, 10 aa, Artificial Sequence
  FAP-FR3-C02 HCDR2
TINSNSDITY

SEQ ID NO: 190 - protein, 10 aa, Artificial Sequence
  FAP-FR3-D02 HCDR2
AISRTGGSTY

SEQ ID NO: 191 - protein, 10 aa, Artificial Sequence
  FAP-FR3-F02 HCDR2
AISRTGGSTY

SEQ ID NO: 192 - protein, 10 aa, Artificial Sequence
  FAP-FR3-G02 HCDR2
TINRFSSITY

SEQ ID NO: 193 - protein, 10 aa, Artificial Sequence
  FAP-FR3-A03 HCDR2
TISSASDITY

SEQ ID NO: 194 - protein, 10 aa, Artificial Sequence
  FAP-FR3-B03 HCDR2
AISRTGGSTY

SEQ ID NO: 195 - protein, 10 aa, Artificial Sequence
  FAP-FR3-C03 HCDR2
AISRTGGSTY

SEQ ID NO: 196 - protein, 10 aa, Artificial Sequence
  FAP-FR3-D03 HCDR2
AISRTGGSTY

SEQ ID NO: 197 - protein, 10 aa, Artificial Sequence
  FAP-FR3-A04 HCDR2
AISRTGGSTY

SEQ ID NO: 198 - protein, 10 aa, Artificial Sequence
  FAP-FR3-B04 HCDR2
AISRTGGSTY

SEQ ID NO: 199 - protein, 10 aa, Artificial Sequence
  FAP-FR3-E04 HCDR2
SINSSDSSTY

SEQ ID NO: 200 - protein, 10 aa, Artificial Sequence
  FAP-FR3-G04 HCDR2
AINSFDDITY

SEQ ID NO: 201 - protein, 10 aa, Artificial Sequence
  FAP-FR3-H04 HCDR2
AISRTGGSTY

SEQ ID NO: 202 - protein, 10 aa, Artificial Sequence
  FAP-FR3-D05 HCDR2
TINALDDITY

SEQ ID NO: 203 - protein, 10 aa, Artificial Sequence
  FAP-FR3-E05 HCDR2
AISSFDDITY

SEQ ID NO: 204 - protein, 10 aa, Artificial Sequence
  FAP-FR3-G05 HCDR2
AISRTGGSTY

SEQ ID NO: 205 - protein, 10 aa, Artificial Sequence
  FAP-FR3-H05 HCDR2
AISRTGGSTY

SEQ ID NO: 206 - protein, 10 aa, Artificial Sequence
  FAP-FR3-C06 HCDR2
AISRTGGSTY

SEQ ID NO: 207 - protein, 10 aa, Artificial Sequence
  FAP-FR3-E06 HCDR2
AISRTGGSTY

SEQ ID NO: 208 - protein, 10 aa, Artificial Sequence
  FAP-FR3-F06 HCDR2
TINSFSDITY

SEQ ID NO: 209 - protein, 10 aa, Artificial Sequence
  FAP-FR3-G06 HCDR2
SINSIDAITY

SEQ ID NO: 210 - protein, 10 aa, Artificial Sequence
  FAP-FR3-H06 HCDR2
AINSIDSITY

SEQ ID NO: 211 - protein, 10 aa, Artificial Sequence
  FAP-FR3-A07 HCDR2
AISRTGGSTY

SEQ ID NO: 212 - protein, 10 aa, Artificial Sequence
  FAP-FR3-B07 HCDR2
SINSFDDITY

SEQ ID NO: 213 - protein, 10 aa, Artificial Sequence
  FAP-FR3-D07 HCDR2
AINPYGDITN

SEQ ID NO: 214 - protein, 10 aa, Artificial Sequence
  FAP-FR3-E07 HCDR2
SINAFDDITY

SEQ ID NO: 215 - protein, 10 aa, Artificial Sequence
  FAP-FR3-F07 HCDR2
SISSFDDITY

SEQ ID NO: 216 - protein, 10 aa, Artificial Sequence
  FAP-FR3-G07 HCDR2
AISRTGGSTY

SEQ ID NO: 217 - protein, 11 aa, Artificial Sequence
  FAP-FR2-A01 HCDR3
AGHWDSHFNYW

SEQ ID NO: 218 - protein, 12 aa, Artificial Sequence
  FAP-FR2-B01 HCDR3
VGDWDIFHYSYW

SEQ ID NO: 219 - protein, 11 aa, Artificial Sequence
  FAP-FR2-C01 HCDR3
AGIVQYHYNYW

SEQ ID NO: 220 - protein, 11 aa, Artificial Sequence
  FAP-FR2-E01 HCDR3
AGEWMMHFNYW

SEQ ID NO: 221 - protein, 11 aa, Artificial Sequence
  FAP-FR2-F01 HCDR3
AGTDTWHYNYW

SEQ ID NO: 222 - protein, 11 aa, Artificial Sequence
  FAP-FR2-G01 HCDR3
AGPWHAHFSYW

SEQ ID NO: 223 - protein, 14 aa, Artificial Sequence
  FAP-FR2-H01 HCDR3
VSTYRADARSLGYW

SEQ ID NO: 224 - protein, 11 aa, Artificial Sequence
  FAP-FR2-B02 HCDR3
AATTQFHFGYW

SEQ ID NO: 225 - protein, 11 aa, Artificial Sequence
  FAP-FR2-C02 HCDR3
AGTFDQHFNYW

SEQ ID NO: 226 - protein, 11 aa, Artificial Sequence
  FAP-FR2-D02 HCDR3
ASRTWMHFNYW

SEQ ID NO: 227 - protein, 11 aa, Artificial Sequence
  FAP-FR2-E02 HCDR3
AGQYQYHYSYW

SEQ ID NO: 228 - protein, 11 aa, Artificial Sequence
  FAP-FR2-H02 HCDR3
ASATKDHYNYW

SEQ ID NO: 229 - protein, 11 aa, Artificial Sequence
  FAP-FR2-B03 HCDR3
AGNIHYHYNYW

SEQ ID NO: 230 - protein, 11 aa, Artificial Sequence
  FAP-FR2-C03 HCDR3
AGESDRHYNYW

SEQ ID NO: 231 - protein, 11 aa, Artificial Sequence
  FAP-FR2-D03 HCDR3
VGYVFQHFNYW

SEQ ID NO: 232 - protein, 11 aa, Artificial Sequence
  FAP-FR2-F03 HCDR3
AAFWDYHYDYW

SEQ ID NO: 233 - protein, 11 aa, Artificial Sequence
  FAP-FR2-H03 HCDR3
AGGTEWHFNYW

SEQ ID NO: 234 - protein, 11 aa, Artificial Sequence
  FAP-FR2-B04 HCDR3
ASVISFHYNYW

SEQ ID NO: 235 - protein, 11 aa, Artificial Sequence
  FAP-FR2-D04 HCDR3
AGPMNWHYGYW

SEQ ID NO: 236 - protein, 11 aa, Artificial Sequence
  FAP-FR2-E04 HCDR3
AGPYYWGLSYW

SEQ ID NO: 237 - protein, 11 aa, Artificial Sequence
  FAP-FR2-G04 HCDR3
VGQYDWHFDYW

SEQ ID NO: 238 - protein, 11 aa, Artificial Sequence
  FAP-FR2-A05 HCDR3
AGQPMYHFNYW

SEQ ID NO: 239 - protein, 17 aa, Artificial Sequence
  FAP-FR2-C05 HCDR3
VVTVWYSPFKVKMYNYW

SEQ ID NO: 240 - protein, 11 aa, Artificial Sequence
  FAP-FR2-D05 HCDR3
AGIWHMHYNYW

SEQ ID NO: 241 - protein, 14 aa, Artificial Sequence
  FAP-FR2-E05 HCDR3
ATSWRSPPPAYGYW

SEQ ID NO: 242 - protein, 11 aa, Artificial Sequence
  FAP-FR2-F05 HCDR3
AGAWKNHFNYW

SEQ ID NO: 243 - protein, 11 aa, Artificial Sequence
  FAP-FR2-G05 HCDR3
AGDSDLHYSYW

SEQ ID NO: 244 - protein, 14 aa, Artificial Sequence
  FAP-FR2-H05 HCDR3
ATSPRGLSAGHGYW

SEQ ID NO: 245 - protein, 11 aa, Artificial Sequence
  FAP-FR2-A06 HCDR3
ATEVPMHYNYW

SEQ ID NO: 246 - protein, 11 aa, Artificial Sequence
  FAP-FR2-B06 HCDR3
AGEWPLHYSYW

SEQ ID NO: 247 - protein, 11 aa, Artificial Sequence
  FAP-FR2-C06 HCDR3
AGLWHQHYNYW

SEQ ID NO: 248 - protein, 11 aa, Artificial Sequence
  FAP-FR2-D06 HCDR3
AGNWTFHFNYW

SEQ ID NO: 249 - protein, 11 aa, Artificial Sequence
  FAP-FR2-E06 HCDR3
AGRYLWHYNYW

SEQ ID NO: 250 - protein, 11 aa, Artificial Sequence
  FAP-FR2-F06 HCDR3
AGLQEYHYNYW

SEQ ID NO: 251 - protein, 11 aa, Artificial Sequence
  FAP-FR2-A07 HCDR3
AGDYDRHYSYW

SEQ ID NO: 252 - protein, 11 aa, Artificial Sequence
  FAP-FR2-B07 HCDR3
AGVEQFHFNYW

SEQ ID NO: 253 - protein, 11 aa, Artificial Sequence
  FAP-FR2-C07 HCDR3
AGHWNHHYNYW

SEQ ID NO: 254 - protein, 11 aa, Artificial Sequence
  FAP-FR2-D07 HCDR3
VGDWDWHFNYW

SEQ ID NO: 255 - protein, 11 aa, Artificial Sequence
  FAP-FR2-E07 HCDR3
AGYYPMHYSYW

SEQ ID NO: 256 - protein, 11 aa, Artificial Sequence
  FAP-FR2-F07 HCDR3
AGDNWMHHNYW

SEQ ID NO: 257 - protein, 11 aa, Artificial Sequence
  FAP-FR2-G07 HCDR3
AGYWFAHFNYW

SEQ ID NO: 258 - protein, 11 aa, Artificial Sequence
  FAP-FR2-H07 HCDR3
AGDHDLHYNYW

SEQ ID NO: 259 - protein, 11 aa, Artificial Sequence
  FAP-FR2-A08 HCDR3
VGLYDRHYNYW

SEQ ID NO: 260 - protein, 12 aa, Artificial Sequence
  FAP-FR2-C08 HCDR3
VGIWDFYHFNYW

SEQ ID NO: 261 - protein, 11 aa, Artificial Sequence
  FAP-FR2-D08 HCDR3
AGDIMYHYNYW

SEQ ID NO: 262 - protein, 14 aa, Artificial Sequence
  FAP-FR2-E08 HCDR3
VKERFTTPYAHSYW

SEQ ID NO: 263 - protein, 14 aa, Artificial Sequence
  FAP-FR2-G08 HCDR3
AGPLWQLYRSHGYW

SEQ ID NO: 264 - protein, 11 aa, Artificial Sequence
  FAP-FR2-A09 HCDR3
VGPVYLHYNYW

SEQ ID NO: 265 - protein, 11 aa, Artificial Sequence
  FAP-FR2-B09 HCDR3
AGRFPFHYDYW

SEQ ID NO: 266 - protein, 14 aa, Artificial Sequence
  FAP-FR2-C09 HCDR3
VSSKGSSAASLNYW

SEQ ID NO: 267 - protein, 11 aa, Artificial Sequence
  FAP-FR2-D09 HCDR3
AGDYYGHHNYW

SEQ ID NO: 268 - protein, 11 aa, Artificial Sequence
  FAP-FR2-E09 HCDR3
AGYVQWHYGYW

SEQ ID NO: 269 - protein, 11 aa, Artificial Sequence
  FAP-FR2-F09 HCDR3
AGTAHQHYDYW

SEQ ID NO: 270 - protein, 14 aa, Artificial Sequence
  FAP-FR2-G09 HCDR3
VTNHMERHASYGYW

SEQ ID NO: 271 - protein, 11 aa, Artificial Sequence
  FAP-FR2-H09 HCDR3
ATRTDWHFNYW

SEQ ID NO: 272 - protein, 11 aa, Artificial Sequence
  FAP-FR2-B10 HCDR3
AGQYNFHYSYW

SEQ ID NO: 273 - protein, 11 aa, Artificial Sequence
  FAP-FR2-C10 HCDR3
AGTTGLHYNYW

SEQ ID NO: 274 - protein, 11 aa, Artificial Sequence
  FAP-FR2-D10 HCDR3
AGTILHHFNYW

SEQ ID NO: 275 - protein, 11 aa, Artificial Sequence
  FAP-FR2-E10 HCDR3
AGPWHFHFGYW

SEQ ID NO: 276 - protein, 11 aa, Artificial Sequence
  FAP-FR2-F10 HCDR3
AGDYPWHYNYW

SEQ ID NO: 277 - protein, 11 aa, Artificial Sequence
  FAP-FR2-G10 HCDR3
AGPAMLHFSYW

SEQ ID NO: 278 - protein, 11 aa, Artificial Sequence
  FAP-FR2-H10 HCDR3
AGMFKYHFNYW

SEQ ID NO: 279 - protein, 11 aa, Artificial Sequence
  FAP-FR2-A11 HCDR3
VIFPEQVYDYW

SEQ ID NO: 280 - protein, 17 aa, Artificial Sequence
  FAP-FR2-C11 HCDR3
VATFRTTNDYYSSHGYW

SEQ ID NO: 281 - protein, 11 aa, Artificial Sequence
  FAP-FR2-D11 HCDR3
AGGSMFHYNYW

SEQ ID NO: 282 - protein, 11 aa, Artificial Sequence
  FAP-FR2-F11 HCDR3
AGRIMMHFNYW

SEQ ID NO: 283 - protein, 11 aa, Artificial Sequence
  FAP-FR2-G11 HCDR3
ASYTAFHYNYW

SEQ ID NO: 284 - protein, 11 aa, Artificial Sequence
  FAP-FR2-H11 HCDR3
AGPWAFHYNYW

SEQ ID NO: 285 - protein, 11 aa, Artificial Sequence
  FAP-FR2-B12 HCDR3
ATDTADHYNYW

SEQ ID NO: 286 - protein, 11 aa, Artificial Sequence
  FAP-FR2-C12 HCDR3
AGMIQFHHNYW

SEQ ID NO: 287 - protein, 11 aa, Artificial Sequence
  FAP-FR2-D12 HCDR3
AGIWFRHYNYW

SEQ ID NO: 288 - protein, 11 aa, Artificial Sequence
  FAP-FR2-F12 HCDR3
ASPTWMHFNYW

SEQ ID NO: 289 - protein, 11 aa, Artificial Sequence
  FAP-FR2-G12 HCDR3
VGATHLHYSYW

SEQ ID NO: 290 - protein, 11 aa, Artificial Sequence
  FAP-FR2-H12 HCDR3
AGDFPHHFNYW

SEQ ID NO: 291 - protein, 10 aa, Artificial Sequence
  FAP-FR3-A01 HCDR3
AGRWDRHYNY

SEQ ID NO: 292 - protein, 11 aa, Artificial Sequence
  FAP-FR3-B01 HCDR3
AGLVAFHYNYW

SEQ ID NO: 293 - protein, 11 aa, Artificial Sequence
  FAP-FR3-E01 HCDR3
AGDFHRHYNYW

SEQ ID NO: 294 - protein, 11 aa, Artificial Sequence
  FAP-FR3-F01 HCDR3
ADIFPLHFSYW

SEQ ID NO: 295 - protein, 11 aa, Artificial Sequence
  FAP-FR3-H01 HCDR3
AGQYEWHYSYW

SEQ ID NO: 296 - protein, 11 aa, Artificial Sequence
  FAP-FR3-A02 HCDR3
AGHAWLHHNYW

SEQ ID NO: 297 - protein, 11 aa, Artificial Sequence
  FAP-FR3-C02 HCDR3
AGPWNHHYNYW

SEQ ID NO: 298 - protein, 11 aa, Artificial Sequence
  FAP-FR3-D02 HCDR3
AGDNFMHHNYW

SEQ ID NO: 299 - protein, 11 aa, Artificial Sequence
  FAP-FR3-F02 HCDR3
AADFPMHFNYW

SEQ ID NO: 300 - protein, 11 aa, Artificial Sequence
  FAP-FR3-G02 HCDR3
ATVTPFHFSYW

SEQ ID NO: 301 - protein, 11 aa, Artificial Sequence
  FAP-FR3-A03 HCDR3
AGIYMWHYNYW

SEQ ID NO: 302 - protein, 11 aa, Artificial Sequence
  FAP-FR3-B03 HCDR3
AGDEDYHYNYW

SEQ ID NO: 303 - protein, 11 aa, Artificial Sequence
  FAP-FR3-C03 HCDR3
AAEFQWHFGYW

SEQ ID NO: 304 - protein, 11 aa, Artificial Sequence
  FAP-FR3-D03 HCDR3
AGHSWLHFNYW

SEQ ID NO: 305 - protein, 11 aa, Artificial Sequence
  FAP-FR3-A04 HCDR3
VGQEHLHYNYW

SEQ ID NO: 306 - protein, 14 aa, Artificial Sequence
  FAP-FR3-B04 HCDR3
ATTYQGRDDGFNYW

SEQ ID NO: 307 - protein, 11 aa, Artificial Sequence
  FAP-FR3-E04 HCDR3
AGDYDRHFNYW

SEQ ID NO: 308 - protein, 11 aa, Artificial Sequence
  FAP-FR3-G04 HCDR3
AGPLYLHHDYW

SEQ ID NO: 309 - protein, 11 aa, Artificial Sequence
  FAP-FR3-H04 HCDR3
AGWWQMHYNYW

SEQ ID NO: 310 - protein, 11 aa, Artificial Sequence
  FAP-FR3-D05 HCDR3
AGTAPFHYNYW

SEQ ID NO: 311 - protein, 23 aa, Artificial Sequence
  FAP-FR3-E05 HCDR3
AHYNRYAPPAADGNTALGTLGYW

SEQ ID NO: 312 - protein, 11 aa, Artificial Sequence
  FAP-FR3-G05 HCDR3
AGHHYHHYNYW

SEQ ID NO: 313 - protein, 17 aa, Artificial Sequence
  FAP-FR3-H05 HCDR3
ASDALAADGVDWHYNYW

SEQ ID NO: 314 - protein, 11 aa, Artificial Sequence
  FAP-FR3-C06 HCDR3
AGDMHHHYNYW

SEQ ID NO: 315 - protein, 11 aa, Artificial Sequence
  FAP-FR3-E06 HCDR3
AGMHGLHYNYW

SEQ ID NO: 316 - protein, 10 aa, Artificial Sequence
  FAP-FR3-F06 HCDR3
VGDWDWLFMY

SEQ ID NO: 317 - protein, 11 aa, Artificial Sequence
  FAP-FR3-G06 HCDR3
AGYAPLHFNYW

SEQ ID NO: 318 - protein, 11 aa, Artificial Sequence
  FAP-FR3-H06 HCDR3
AGYSTFHYNYW

SEQ ID NO: 319 - protein, 11 aa, Artificial Sequence
  FAP-FR3-A07 HCDR3
AGDINYHYNYW

SEQ ID NO: 320 - protein, 11 aa, Artificial Sequence
  FAP-FR3-B07 HCDR3
AGPWNLHFNYW

SEQ ID NO: 321 - protein, 11 aa, Artificial Sequence
  FAP-FR3-D07 HCDR3
ATFTPGHYNYW

SEQ ID NO: 322 - protein, 11 aa, Artificial Sequence
  FAP-FR3-E07 HCDR3
AGDYIFHFNYW

SEQ ID NO: 323 - protein, 11 aa, Artificial Sequence
  FAP-FR3-F07 HCDR3
AIAHQQKLSYW

SEQ ID NO: 324 - protein, 11 aa, Artificial Sequence
  FAP-FR3-G07 HCDR3
AGETAYHFNYW

SEQ ID NO: 325 - protein, 118 aa, Artificial Sequence
  FAP-FR2-A01 VH
EVQLVESGGGLVQPGGSLRLSCAASGSTSNNYFMGWFRQAPGKGRELVAAINPLDSNTYYPDSVEGRFTISRDNAKRMVYLQMNSLRAEDTAVYYCAAGHWDSHFNYWGQGTQVTVSS

SEQ ID NO: 326 - protein, 119 aa, Artificial Sequence
  FAP-FR2-B01 VH
EVQLVESGGGLVQPGGSLRLSCAASGFTSGVYFMGWFRQAPGKGRELVAAISRTGGSTYYPDSVEGRFTISRDNAKRMVYLQMNSLRAEDTAVYYCAVGDWDIFHYSYWGQGTQVTVSS

SEQ ID NO: 327 - protein, 118 aa, Artificial Sequence
  FAP-FR2-C01 VH
EVQLVESGGGLVQPGGSLRLSCAASGFTFGFNYMGWFRQAPGKGRELVAAISRISDSTYYPDSVEGRFTISRDNAKRMVYLQMNSLRAEDTAVYYCAAGIVQYHYNYWGQGTQVTVSS

SEQ ID NO: 328 - protein, 118 aa, Artificial Sequence
  FAP-FR2-E01 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGEWMMHFNYWGQGTQVTVSS

SEQ ID NO: 329 - protein, 118 aa, Artificial Sequence
  FAP-FR2-F01 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGTDTWHYNYWGQGTQVTVSS

SEQ ID NO: 330 - protein, 118 aa, Artificial Sequence
  FAP-FR2-G01 VH
EVQLVESGGGLVQPGGSLRLSCAASGSIFSHYSMGWFRQAPGKGRELVAAISRTGGSTYYPDSVEGRFTISRDNAKRMVYLQMNSLRAEDTAVYYCAAGPWHAHFSYWGQGTQVTVSS

SEQ ID NO: 331 - protein, 121 aa, Artificial Sequence
  FAP-FR2-H01 VH
EVQLVESGGGLVQPGGSLRLSCAASGYILRDDPMGWFRQAPGKGRELVAAISRTGGSTYYPDSVEGRFTISRDNAKRMVYLQMNSLRAEDTAVYYCAVSTYRADARSLGYWGQGTQVTVSS

SEQ ID NO: 332 - protein, 118 aa, Artificial Sequence
  FAP-FR2-B02 VH
EVQLVESGGGLVQPGGSLRLSCAASGSTFSYYFMGWFRQAPGKGRELVAAISRTGGSTYYPDSVEGRFTISRDNAKRMVYLQMNSLRAEDTAVYYCAAATTQFHFGYWGQGTQVTVSS

SEQ ID NO: 333 - protein, 118 aa, Artificial Sequence
  FAP-FR2-C02 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGTFDQHFNYWGQGTQVTVSS

SEQ ID NO: 334 - protein, 118 aa, Artificial Sequence
  FAP-FR2-D02 VH
EVQLVESGGGLVQPGGSLRLSCAASGFIYSNYYMGWFRQAPGKGRELVAAISRTGGSTYYPDSVEGRFTISRDNAKRMVYLQMNSLRAEDTAVYYCAASRTWMHFNYWGQGTQVTVSS

SEQ ID NO: 335 - protein, 118 aa, Artificial Sequence
  FAP-FR2-E02 VH
EVQLVESGGGLVQPGGSLRLSCAASGFIFSAYYMGWFRQAPGKGRELVATINSVDDITYYPDSVEGRFTISRDNAKRMVYLQMNSLRAEDTAVYYCAAGQYQYHYSYWGQGTQVTVSS

SEQ ID NO: 336 - protein, 118 aa, Artificial Sequence
  FAP-FR2-H02 VH
EVQLVESGGGLVQPGGSLRLSCAASGSTYDVDYMGWFRQAPGKGRELVATINSYGGITYYPDSVEGRFTISRDNAKRMVYLQMNSLRAEDTAVYYCAASATKDHYNYWGQGTQVTVSS

SEQ ID NO: 337 - protein, 118 aa, Artificial Sequence
  FAP-FR2-B03 VH
EVQLVESGGGLVQPGGSLRLSCAASGSIYSFYFMGWFRQAPGKGRELVAAISRTGGSTYYPDSVEGRFTISRDNAKRMVYLQMNSLRAEDTAVYYCAAGNIHYHYNYWGQGTQVTVSS

SEQ ID NO: 338 - protein, 118 aa, Artificial Sequence
  FAP-FR2-C03 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGESDRHYNYWGQGTQVTVSS

SEQ ID NO: 339 - protein, 118 aa, Artificial Sequence
  FAP-FR2-D03 VH
EVQLVESGGGLVQPGGSLRLSCAASGRIYRNYSMGWFRQAPGKGRELVAAISRTGGSTYYPDSVEGRFTISRDNAKRMVYLQMNSLRAEDTAVYYCAVGYVFQHFNYWGQGTQVTVSS

SEQ ID NO: 340 - protein, 118 aa, Artificial Sequence
  FAP-FR2-F03 VH
EVQLVESGGGLVQPGGSLRLSCAASGFIFSFYFMGWFRQAPGKGRELVAAISRTGGSTYYPDSVEGRFTISRDNAKRMVYLQMNSLRAEDTAVYYCAAAFWDYHYDYWGQGTQVTVSS

SEQ ID NO: 341 - protein, 118 aa, Artificial Sequence
  FAP-FR2-H03 VH
EVQLVESGGGLVQPGGSLRLSCAASGYIFSVNFMGWFRQAPGKGRELVATINWFDDITYYPDSVEGRFTISRDNAKRMVYLQMNSLRAEDTAVYYCAAGGTEWHFNYWGQGTQVTVSS

SEQ ID NO: 342 - protein, 118 aa, Artificial Sequence
  FAP-FR2-B04 VH
EVQLVESGGGLVQPGGSLRLSCAASGSIFDYYYMGWFRQAPGKGRELVAAISRTGGSTYYPDSVEGRFTISRDNAKRMVYLQMNSLRAEDTAVYYCAASVISFHYNYWGQGTQVTVSS

SEQ ID NO: 343 - protein, 118 aa, Artificial Sequence
  FAP-FR2-D04 VH
EVQLVESGGGLVQPGGSLRLSCAASGSIYSNYYMGWFRQAPGKGRELVAAISRTGGSTYYPDSVEGRFTISRDNAKRMVYLQMNSLRAEDTAVYYCAAGPMNWHYGYWGQGTQVTVSS

SEQ ID NO: 344 - protein, 118 aa, Artificial Sequence
  FAP-FR2-E04 VH
EVQLVESGGGLVQPGGSLRLSCAASGFIFNIYSMGWFRQAPGKGRELVASINWSDSSTYYPDSVEGRFTISRDNAKRMVYLQMNSLRAEDTAVYYCAAGPYYWGLSYWGQGTQVTVSS

SEQ ID NO: 345 - protein, 118 aa, Artificial Sequence
  FAP-FR2-G04 VH
EVQLVESGGGLVQPGGSLRLSCAASGSIYDLYTMGWFRQAPGKGRELVAAISRTGGSTYYPDSVEGRFTISRDNAKRMVYLQMNSLRAEDTAVYYCAVGQYDWHFDYWGQGTQVTVSS

SEQ ID NO: 346 - protein, 118 aa, Artificial Sequence
  FAP-FR2-A05 VH
EVQLVESGGGLVQPGGSLRLSCAASGRIYDLYNMGWFRQAPGKGRELVAAISRTGGSTYYPDSVEGRFTISRDNAKRMVYLQMNSLRAEDTAVYYCAAGQPMYHFNYWGQGTQVTVSS

SEQ ID NO: 347 - protein, 124 aa, Artificial Sequence
  FAP-FR2-C05 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAVVTVWYSPFKVKMYNYWGQGTQVTVSS

SEQ ID NO: 348 - protein, 118 aa, Artificial Sequence
  FAP-FR2-D05 VH
EVQLVESGGGLVQPGGSLRLSCAASGFTYGFNYMGWFRQAPGKGRELVAAISRTGGSTYYPDSVEGRFTISRDNAKRMVYLQMNSLRAEDTAVYYCAAGIWHMHYNYWGQGTQVTVSS

SEQ ID NO: 349 - protein, 121 aa, Artificial Sequence
  FAP-FR2-E05 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAATSWRSPPPAYGYWGQGTQVTVSS

SEQ ID NO: 350 - protein, 118 aa, Artificial Sequence
  FAP-FR2-F05 VH
EVQLVESGGGLVQPGGSLRLSCAASGSIYRFDSMGWFRQAPGKGRELVATITSLDSITYYPDSVEGRFTISRDNAKRMVYLQMNSLRAEDTAVYYCAAGAWKNHFNYWGQGTQVTVSS

SEQ ID NO: 351 - protein, 118 aa, Artificial Sequence
  FAP-FR2-G05 VH
EVQLVESGGGLVQPGGSLRLSCAASGFISSAYSMGWFRQAPGKGRELVAAISRTGGSTYYPDSVEGRFTISRDNAKRMVYLQMNSLRAEDTAVYYCAAGDSDLHYSYWGQGTQVTVSS

SEQ ID NO: 352 - protein, 121 aa, Artificial Sequence
  FAP-FR2-H05 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAATSPRGLSAGHGYWGQGTQVTVSS

SEQ ID NO: 353 - protein, 118 aa, Artificial Sequence
  FAP-FR2-A06 VH
EVQLVESGGGLVQPGGSLRLSCAASGSIFSSYIMGWFRQAPGKGRELVASINSYDSTTYYPDSVEGRFTISRDNAKRMVYLQMNSLRAEDTAVYYCAATEVPMHYNYWGQGTQVTVSS

SEQ ID NO: 354 - protein, 118 aa, Artificial Sequence
  FAP-FR2-B06 VH
EVQLVESGGGLVQPGGSLRLSCAASGFTLNIYHMGWFRQAPGKGRELVAAINSFSDITYYPDSVEGRFTISRDNAKRMVYLQMNSLRAEDTAVYYCAAGEWPLHYSYWGQGTQVTVSS

SEQ ID NO: 355 - protein, 118 aa, Artificial Sequence
  FAP-FR2-C06 VH
EVQLVESGGGLVQPGGSLRLSCAASGGTFSFYFMGWFRQAPGKGRELVATINSFDDITYYPDSVEGRFTISRDNAKRMVYLQMNSLRAEDTAVYYCAAGLWHQHYNYWGQGTQVTVSS

SEQ ID NO: 356 - protein, 118 aa, Artificial Sequence
  FAP-FR2-D06 VH
EVQLVESGGGLVQPGGSLRLSCAASGRIYDANFMGWFRQAPGKGRELVATINTYSDITYYPDSVEGRFTISRDNAKRMVYLQMNSLRAEDTAVYYCAAGNWTFHFNYWGQGTQVTVSS

SEQ ID NO: 357 - protein, 118 aa, Artificial Sequence
  FAP-FR2-E06 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGRYLWHYNYWGQGTQVTVSS

SEQ ID NO: 358 - protein, 118 aa, Artificial Sequence
  FAP-FR2-F06 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGLQEYHYNYWGQGTQVTVSS

SEQ ID NO: 359 - protein, 118 aa, Artificial Sequence
  FAP-FR2-A07 VH
EVQLVESGGGLVQPGGSLRLSCAASGFIFDYYFMGWFRQAPGKGRELVATINSFSSSTYYPDSVEGRFTISRDNAKRMVYLQMNSLRAEDTAVYYCAAGDYDRHYSYWGQGTQVTVSS

SEQ ID NO: 360 - protein, 117 aa, Artificial Sequence
  FAP-FR2-B07 VH
EVQLVESGGGLVQPGGSLRLSCAASGRTFSYNPMGWFRQAPGKGRELVATISFSGITYYPDSVEGRFTISRDNAKRMVYLQMNSLRAEDTAVYYCAAGVEQFHFNYWGQGTQVTVSS

SEQ ID NO: 361 - protein, 118 aa, Artificial Sequence
  FAP-FR2-C07 VH
EVQLVESGGGLVQPGGSLRLSCAASGFTFSFYFMGWFRQAPGKGRELVASINAYSDSTYYPDSVEGRFTISRDNAKRMVYLQMNSLRAEDTAVYYCAAGHWNHHYNYWGQGTQVTVSS

SEQ ID NO: 362 - protein, 118 aa, Artificial Sequence
  FAP-FR2-D07 VH
EVQLVESGGGLVQPGGSLRLSCAASGRIFSSYSMGWFRQAPGKGRELVATISSFSDITYYPDSVEGRFTISRDNAKRMVYLQMNSLRAEDTAVYYCAVGDWDWHFNYWGQGTQVTVSS

SEQ ID NO: 363 - protein, 118 aa, Artificial Sequence
  FAP-FR2-E07 VH
EVQLVESGGGLVQPGGSLRLSCAASGSTFGYDNMGWFRQAPGKGRELVATINSFGDITNYPDSVEGRFTISRDNAKRMVYLQMNSLRAEDTAVYYCAAGYYPMHYSYWGQGTQVTVSS

SEQ ID NO: 364 - protein, 118 aa, Artificial Sequence
  FAP-FR2-F07 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGDNWMHHNYWGQGTQVTVSS

SEQ ID NO: 365 - protein, 118 aa, Artificial Sequence
  FAP-FR2-G07 VH
EVQLVESGGGLVQPGGSLRLSCAASGGISDYYSMGWFRQAPGKGRELVAAISRTGGSTYYPDSVEGRFTISRDNAKRMVYLQMNSLRAEDTAVYYCAAGYWFAHFNYWGQGTQVTVSS

SEQ ID NO: 366 - protein, 118 aa, Artificial Sequence
  FAP-FR2-H07 VH
EVQLVESGGGLVQPGGSLRLSCAASGYIFSFYFMGWFRQAPGKGRELVAAISRTGGSTYYPDSVEGRFTISRDNAKRMVYLQMNSLRAEDTAVYYCAAGDHDLHYNYWGQGTQVTVSS

SEQ ID NO: 367 - protein, 118 aa, Artificial Sequence
  FAP-FR2-A08 VH
EVQLVESGGGLVQPGGSLRLSCAASGFIFGIYFMGWFRQAPGKGRELVAAISRTGGSTYYPDSVEGRFTISRDNAKRMVYLQMNSLRAEDTAVYYCAVGLYDRHYNYWGQGTQVTVSS

SEQ ID NO: 368 - protein, 119 aa, Artificial Sequence
  FAP-FR2-C08 VH
EVQLVESGGGLVQPGGSLRLSCAASGSIYGYDYMGWFRQAPGKGRELVAAISRTGGSTYYPDSVEGRFTISRDNAKRMVYLQMNSLRAEDTAVYYCAVGIWDFYHFNYWGQGTQVTVSS

SEQ ID NO: 369 - protein, 118 aa, Artificial Sequence
  FAP-FR2-D08 VH
EVQLVESGGGLVQPGGSLRLSCAASGSILGSYIMGWFRQAPGKGRELVATINSNDSITYYPDSVEGRFTISRDNAKRMVYLQMNSLRAEDTAVYYCAAGDIMYHYNYWGQGTQVTVSS

SEQ ID NO: 370 - protein, 121 aa, Artificial Sequence
  FAP-FR2-E08 VH
EVQLVESGGGLVQPGGSLRLSCAASGFIYSFYVMGWFRQAPGKGRELVATINSFDDTTYYPDSVEGRFTISRDNAKRMVYLQMNSLRAEDTAVYYCAVKERFTTPYAHSYWGQGTQVTVSS

SEQ ID NO: 371 - protein, 121 aa, Artificial Sequence
  FAP-FR2-G08 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGPLWQLYRSHGYWGQGTQVTVSS

SEQ ID NO: 372 - protein, 118 aa, Artificial Sequence
  FAP-FR2-A09 VH
EVQLVESGGGLVQPGGSLRLSCAASGFTFSFYSMGWFRQAPGKGRELVASINAIDSITYYPDSVEGRFTISRDNAKRMVYLQMNSLRAEDTAVYYCAVGPVYLHYNYWGQGTQVTVSS

SEQ ID NO: 373 - protein, 118 aa, Artificial Sequence
  FAP-FR2-B09 VH
EVQLVESGGGLVQPGGSLRLSCAASGGTFGVNPMGWFRQAPGKGRELVATITSTDDITYYPDSVEGRFTISRDNAKRMVYLQMNSLRAEDTAVYYCAAGRFPFHYDYWGQGTQVTVSS

SEQ ID NO: 374 - protein, 121 aa, Artificial Sequence
  FAP-FR2-C09 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAVSSKGSSAASLNYWGQGTQVTVSS

SEQ ID NO: 375 - protein, 118 aa, Artificial Sequence
  FAP-FR2-D09 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGDYYGHHNYWGQGTQVTVSS

SEQ ID NO: 376 - protein, 118 aa, Artificial Sequence
  FAP-FR2-E09 VH
EVQLVESGGGLVQPGGSLRLSCAASGFISGHYYMGWFRQAPGKGRELVAAISRTGGSTYYPDSVEGRFTISRDNAKRMVYLQMNSLRAEDTAVYYCAAGYVQWHYGYWGQGTQVTVSS

SEQ ID NO: 377 - protein, 118 aa, Artificial Sequence
  FAP-FR2-F09 VH
EVQLVESGGGLVQPGGSLRLSCAASGGILRIYFMGWFRQAPGKGRELVAAISRTGGSTYYPDSVEGRFTISRDNAKRMVYLQMNSLRAEDTAVYYCAAGTAHQHYDYWGQGTQVTVSS

SEQ ID NO: 378 - protein, 121 aa, Artificial Sequence
  FAP-FR2-G09 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAVTNHMERHASYGYWGQGTQVTVSS

SEQ ID NO: 379 - protein, 118 aa, Artificial Sequence
  FAP-FR2-H09 VH
EVQLVESGGGLVQPGGSLRLSCAASGFIFNVYYMGWFRQAPGKGRELVATISWYSDITYYPDSVEGRFTISRDNAKRMVYLQMNSLRAEDTAVYYCAATRTDWHFNYWGQGTQVTVSS

SEQ ID NO: 380 - protein, 118 aa, Artificial Sequence
  FAP-FR2-B10 VH
EVQLVESGGGLVQPGGSLRLSCAASGSIYSFYFMGWFRQAPGKGRELVATINSFDSITYYPDSVEGRFTISRDNAKRMVYLQMNSLRAEDTAVYYCAAGQYNFHYSYWGQGTQVTVSS

SEQ ID NO: 381 - protein, 118 aa, Artificial Sequence
  FAP-FR2-C10 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGTTGLHYNYWGQGTQVTVSS

SEQ ID NO: 382 - protein, 118 aa, Artificial Sequence
  FAP-FR2-D10 VH
EVQLVESGGGLVQPGGSLRLSCAASGSIFGAYYMGWFRQAPGKGRELVAAINPDSDITYYPDSVEGRFTISRDNAKRMVYLQMNSLRAEDTAVYYCAAGTILHHFNYWGQGTQVTVSS

SEQ ID NO: 383 - protein, 118 aa, Artificial Sequence
  FAP-FR2-E10 VH
EVQLVESGGGLVQPGGSLRLSCAASGSTFRFYSMGWFRQAPGKGRELVATISSFDDITYYPDSVEGRFTISRDNAKRMVYLQMNSLRAEDTAVYYCAAGPWHFHFGYWGQGTQVTVSS

SEQ ID NO: 384 - protein, 118 aa, Artificial Sequence
  FAP-FR2-F10 VH
EVQLVESGGGLVQPGGSLRLSCAASGFILGSDTMGWFRQAPGKGRELVASINSFDSITYYPDSVEGRFTISRDNAKRMVYLQMNSLRAEDTAVYYCAAGDYPWHYNYWGQGTQVTVSS

SEQ ID NO: 385 - protein, 118 aa, Artificial Sequence
  FAP-FR2-G10 VH
EVQLVESGGGLVQPGGSLRLSCAASGFIFSVYSMGWFRQAPGKGRELVAAINTVDSITYYPDSVEGRFTISRDNAKRMVYLQMNSLRAEDTAVYYCAAGPAMLHFSYWGQGTQVTVSS

SEQ ID NO: 386 - protein, 118 aa, Artificial Sequence
  FAP-FR2-H10 VH
EVQLVESGGGLVQPGGSLRLSCAASGRTFNFYAMGWFRQAPGKGRELVAAISRTGGSTYYPDSVEGRFTISRDNAKRMVYLQMNSLRAEDTAVYYCAAGMFKYHFNYWGQGTQVTVSS

SEQ ID NO: 387 - protein, 118 aa, Artificial Sequence
  FAP-FR2-A11 VH
EVQLVESGGGLVQPGGSLRLSCAASGRTLSSYTMGWFRQAPGKGRELVASISWVDSITYYPDSVEGRFTISRDNAKRMVYLQMNSLRAEDTAVYYCAVIFPEQVYDYWGQGTQVTVSS

SEQ ID NO: 388 - protein, 124 aa, Artificial Sequence
  FAP-FR2-C11 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAVATFRTTNDYYSSHGYWGQGTQVTVSS

SEQ ID NO: 389 - protein, 118 aa, Artificial Sequence
  FAP-FR2-D11 VH
EVQLVESGGGLVQPGGSLRLSCAASGYISSSYSMGWFRQAPGKGRELVAAISSFSDITYYPDSVEGRFTISRDNAKRMVYLQMNSLRAEDTAVYYCAAGGSMFHYNYWGQGTQVTVSS

SEQ ID NO: 390 - protein, 118 aa, Artificial Sequence
  FAP-FR2-F11 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGRIMMHFNYWGQGTQVTVSS

SEQ ID NO: 391 - protein, 118 aa, Artificial Sequence
  FAP-FR2-G11 VH
EVQLVESGGGLVQPGGSLRLSCAASGSIYSHNFMGWFRQAPGKGRELVAAISRTGGSTYYPDSVEGRFTISRDNAKRMVYLQMNSLRAEDTAVYYCAASYTAFHYNYWGQGTQVTVSS

SEQ ID NO: 392 - protein, 118 aa, Artificial Sequence
  FAP-FR2-H11 VH
EVQLVESGGGLVQPGGSLRLSCAASGFIFSFYSMGWFRQAPGKGRELVATINTFSDITYYPDSVEGRFTISRDNAKRMVYLQMNSLRAEDTAVYYCAAGPWAFHYNYWGQGTQVTVSS

SEQ ID NO: 393 - protein, 118 aa, Artificial Sequence
  FAP-FR2-B12 VH
EVQLVESGGGLVQPGGSLRLSCAASGFIFDFYFMGWFRQAPGKGRELVATINSISDITYYPDSVEGRFTISRDNAKRMVYLQMNSLRAEDTAVYYCAATDTADHYNYWGQGTQVTVSS

SEQ ID NO: 394 - protein, 118 aa, Artificial Sequence
  FAP-FR2-C12 VH
EVQLVESGGGLVQPGGSLRLSCAASGFIYSSNNMGWFRQAPGKGRELVASINSFDDSTYYPDSVEGRFTISRDNAKRMVYLQMNSLRAEDTAVYYCAAGMIQFHHNYWGQGTQVTVSS

SEQ ID NO: 395 - protein, 118 aa, Artificial Sequence
  FAP-FR2-D12 VH
EVQLVESGGGLVQPGGSLRLSCAASGRILRHYHMGWFRQAPGKGRELVATINTLDSITYYPDSVEGRFTISRDNAKRMVYLQMNSLRAEDTAVYYCAAGIWFRHYNYWGQGTQVTVSS

SEQ ID NO: 396 - protein, 118 aa, Artificial Sequence
  FAP-FR2-F12 VH
EVQLVESGGGLVQPGGSLRLSCAASGFIFGSYFMGWFRQAPGKGRELVAAISRTGGSTYYPDSVEGRFTISRDNAKRMVYLQMNSLRAEDTAVYYCAASPTWMHFNYWGQGTQVTVSS

SEQ ID NO: 397 - protein, 118 aa, Artificial Sequence
  FAP-FR2-G12 VH
EVQLVESGGGLVQPGGSLRLSCAASGYIFSSYAMGWFRQAPGKGRELVAAISRTGGSTYYPDSVEGRFTISRDNAKRMVYLQMNSLRAEDTAVYYCAVGATHLHYSYWGQGTQVTVSS

SEQ ID NO: 398 - protein, 118 aa, Artificial Sequence
  FAP-FR2-H12 VH
EVQLVESGGGLVQPGGSLRLSCAASGFTFGAYHMGWFRQAPGKGRELVATINWFGGITYYPDSVEGRFTISRDNAKRMVYLQMNSLRAEDTAVYYCAAGDFPHHFNYWGQGTQVTVSS

SEQ ID NO: 399 - protein, 118 aa, Artificial Sequence
  FAP-FR3-A01 VH
EVQLVESGGGLVQPGGSLRLSCAASGRTFGLDFMGWFRQAPGKGRELVASINSFDDITNYPDSVEGRFTISRDNAKRMVYLQMNSLRAEDTAVYYCAAGRWDRHYNYWGQGTQVTVSS

SEQ ID NO: 400 - protein, 118 aa, Artificial Sequence
  FAP-FR3-B01 VH
EVQLVESGGGLVQPGGSLRLSCAASGYILSFYYMGWFRQAPGKGRELVATINSFSDITYYPDSVEGRFTISRDNAKRMVYLQMNSLRAEDTAVYYCAAGLVAFHYNYWGQGTQVTVSS

SEQ ID NO: 401 - protein, 118 aa, Artificial Sequence
  FAP-FR3-E01 VH
EVQLVESGGGLVQPGGSLRLSCAASGFISSLYSMGWFRQAPGKGRELVATITGSSSITYYPDSVEGRFTISRDNAKRMVYLQMNSLRAEDTAVYYCAAGDFHRHYNYWGQGTQVTVSS

SEQ ID NO: 402 - protein, 118 aa, Artificial Sequence
  FAP-FR3-F01 VH
EVQLVESGGGLVQPGGSLRLSCAASGFIFNYYYMGWFRQAPGKGRELVATISAFDDSTYYPDSVEGRFTISRDNAKRMVYLQMNSLRAEDTAVYYCAADIFPLHFSYWGQGTQVTVSS

SEQ ID NO: 403 - protein, 118 aa, Artificial Sequence
  FAP-FR3-H01 VH
EVQLVESGGGLVQPGGSLRLSCAASGSTSGAYNMGWFRQAPGKGRELVATINWFDDITYYPDSVEGRFTISRDNAKRMVYLQMNSLRAEDTAVYYCAAGQYEWHYSYWGQGTQVTVSS

SEQ ID NO: 404 - protein, 118 aa, Artificial Sequence
  FAP-FR3-A02 VH
EVQLVESGGGLVQPGGSLRLSCAASGFIYNFNSMGWFRQAPGKGRELVAAINRFDDITYYPDSVEGRFTISRDNAKRMVYLQMNSLRAEDTAVYYCAAGHAWLHHNYWGQGTQVTVSS

SEQ ID NO: 405 - protein, 118 aa, Artificial Sequence
  FAP-FR3-C02 VH
EVQLVESGGGLVQPGGSLRLSCAASGSTSRDYSMGWFRQAPGKGRELVATINSNSDITYYPDSVEGRFTISRDNAKRMVYLQMNSLRAEDTAVYYCAAGPWNHHYNYWGQGTQVTVSS

SEQ ID NO: 406 - protein, 118 aa, Artificial Sequence
  FAP-FR3-D02 VH
EVQLVESGGGLVQPGGSLRLSCAASGSTYNVYPMGWFRQAPGKGRELVAAISRTGGSTYYPDSVEGRFTISRDNAKRMVYLQMNSLRAEDTAVYYCAAGDNFMHHNYWGQGTQVTVSS

SEQ ID NO: 407 - protein, 118 aa, Artificial Sequence
  FAP-FR3-F02 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAADFPMHFNYWGQGTQVTVSS

SEQ ID NO: 408 - protein, 118 aa, Artificial Sequence
  FAP-FR3-G02 VH
EVQLVESGGGLVQPGGSLRLSCAASGSTFNYYDMGWFRQAPGKGRELVATINRFSSITYYPDSVEGRFTISRDNAKRMVYLQMNSLRAEDTAVYYCAATVTPFHFSYWGQGTQVTVSS

SEQ ID NO: 409 - protein, 118 aa, Artificial Sequence
  FAP-FR3-A03 VH
EVQLVESGGGLVQPGGSLRLSCAASGFISGVYFMGWFRQAPGKGRELVATISSASDITYYPDSVEGRFTISRDNAKRMVYLQMNSLRAEDTAVYYCAAGIYMWHYNYWGQGTQVTVSS

SEQ ID NO: 410 - protein, 118 aa, Artificial Sequence
  FAP-FR3-B03 VH
EVQLVESGGGLVQPGGSLRLSCAASGFILGANPMGWFRQAPGKGRELVAAISRTGGSTYYPDSVEGRFTISRDNAKRMVYLQMNSLRAEDTAVYYCAAGDEDYHYNYWGQGTQVTVSS

SEQ ID NO: 411 - protein, 118 aa, Artificial Sequence
  FAP-FR3-C03 VH
EVQLVESGGGLVQPGGSLRLSCAASGFIYGYNHMGWFRQAPGKGRELVAAISRTGGSTYYPDSVEGRFTISRDNAKRMVYLQMNSLRAEDTAVYYCAAAEFQWHFGYWGQGTQVTVSS

SEQ ID NO: 412 - protein, 118 aa, Artificial Sequence
  FAP-FR3-D03 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGHSWLHFNYWGQGTQVTVSS

SEQ ID NO: 413 - protein, 118 aa, Artificial Sequence
  FAP-FR3-A04 VH
EVQLVESGGGLVQPGGSLRLSCAASGFIYNYNHMGWFRQAPGKGRELVAAISRTGGSTYYPDSVEGRFTISRDNAKRMVYLQMNSLRAEDTAVYYCAVGQEHLHYNYWGQGTQVTVSS

SEQ ID NO: 414 - protein, 121 aa, Artificial Sequence
  FAP-FR3-B04 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAATTYQGRDDGFNYWGQGTQVTVSS

SEQ ID NO: 415 - protein, 118 aa, Artificial Sequence
  FAP-FR3-E04 VH
EVQLVESGGGLVQPGGSLRLSCAASGSTYSFNTMGWFRQAPGKGRELVASINSSDSSTYYPDSVEGRFTISRDNAKRMVYLQMNSLRAEDTAVYYCAAGDYDRHFNYWGQGTQVTVSS

SEQ ID NO: 416 - protein, 118 aa, Artificial Sequence
  FAP-FR3-G04 VH
EVQLVESGGGLVQPGGSLRLSCAASGSTFSAYSMGWFRQAPGKGRELVAAINSFDDITYYPDSVEGRFTISRDNAKRMVYLQMNSLRAEDTAVYYCAAGPLYLHHDYWGQGTQVTVSS

SEQ ID NO: 417 - protein, 118 aa, Artificial Sequence
  FAP-FR3-H04 VH
EVQLVESGGGLVQPGGSLRLSCAASGSTFNPYFMGWFRQAPGKGRELVAAISRTGGSTYYPDSVEGRFTISRDNAKRMVYLQMNSLRAEDTAVYYCAAGWWQMHYNYWGQGTQVTVSS

SEQ ID NO: 418 - protein, 118 aa, Artificial Sequence
  FAP-FR3-D05 VH
EVQLVESGGGLVQPGGSLRLSCAASGFIFSLYSMGWFRQAPGKGRELVATINALDDITYYPDSVEGRFTISRDNAKRMVYLQMNSLRAEDTAVYYCAAGTAPFHYNYWGQGTQVTVSS

SEQ ID NO: 419 - protein, 130 aa, Artificial Sequence
  FAP-FR3-E05 VH
EVQLVESGGGLVQPGGSLRLSCAASGFIFGYYSMGWFRQAPGKGRELVAAISSFDDITYYPDSVEGRFTISRDNAKRMVYLQMNSLRAEDTAVYYCAAHYNRYAPPAADGNTALGTLGYWGQGTQVTVSS

SEQ ID NO: 420 - protein, 118 aa, Artificial Sequence
  FAP-FR3-G05 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGHHYHHYNYWGQGTQVTVSS

SEQ ID NO: 421 - protein, 124 aa, Artificial Sequence
  FAP-FR3-H05 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAASDALAADGVDWHYNYWGQGTQVTVSS

SEQ ID NO: 422 - protein, 118 aa, Artificial Sequence
  FAP-FR3-C06 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGDMHHHYNYWGQGTQVTVSS

SEQ ID NO: 423 - protein, 118 aa, Artificial Sequence
  FAP-FR3-E06 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGMHGLHYNYWGQGTQVTVSS

SEQ ID NO: 424 - protein, 118 aa, Artificial Sequence
  FAP-FR3-F06 VH
EVQLVESGGGLVQPGGSLRLSCAASGRIFSSYSMGWFRQAPGKGRELVATINSFSDITYYPDSVEGRFTISRDNAKRMVYLQMNSLRAEDTAVYYCAVGDWDWLFMYWGQGTKVTVSS

SEQ ID NO: 425 - protein, 118 aa, Artificial Sequence
  FAP-FR3-G06 VH
EVQLVESGGGLVQPGGSLRLSCAASGGISSVYAMGWFRQAPGKGRELVASINSIDAITYYPDSVEGRFTISRDNAKRMVYLQMNSLRAEDTAVYYCAAGYAPLHFNYWGQGTQVTVSS

SEQ ID NO: 426 - protein, 118 aa, Artificial Sequence
  FAP-FR3-H06 VH
EVQLVESGGGLVQPGGSLRLSCAASGRTLNVDHMGWFRQAPGKGRELVAAINSIDSITYYPDSVEGRFTISRDNAKRMVYLQMNSLRAEDTAVYYCAAGYSTFHYNYWGQGTQVTVSS

SEQ ID NO: 427 - protein, 118 aa, Artificial Sequence
  FAP-FR3-A07 VH
EVQLVESGGGLVQPGGSLRLSCAASGFISSYYYMGWFRQAPGKGRELVAAISRTGGSTYYPDSVEGRFTISRDNAKRMVYLQMNSLRAEDTAVYYCAAGDINYHYNYWGQGTQVTVSS

SEQ ID NO: 428 - protein, 118 aa, Artificial Sequence
  FAP-FR3-B07 VH
EVQLVESGGGLVQPGGSLRLSCAASGYIYSYNFMGWFRQAPGKGRELVASINSFDDITYYPDSVEGRFTISRDNAKRMVYLQMNSLRAEDTAVYYCAAGPWNLHFNYWGQGTQVTVSS

SEQ ID NO: 429 - protein, 118 aa, Artificial Sequence
  FAP-FR3-D07 VH
EVQLVESGGGLVQPGGSLRLSCAASGYTFRIYFMGWFRQAPGKGRELVAAINPYGDITNYPDSVEGRFTISRDNAKRMVYLQMNSLRAEDTAVYYCAATFTPGHYNYWGQGTQVTVSS

SEQ ID NO: 430 - protein, 118 aa, Artificial Sequence
  FAP-FR3-E07 VH
EVQLVESGGGLVQPGGSLRLSCAASGFIYRYYSMGWFRQAPGKGRELVASINAFDDITYYPDSVEGRFTISRDNAKRMVYLQMNSLRAEDTAVYYCAAGDYIFHFNYWGQGTQVTVSS

SEQ ID NO: 431 - protein, 118 aa, Artificial Sequence
  FAP-FR3-F07 VH
EVQLVESGGGLVQPGGSLRLSCAASGSIFDFNHMGWFRQAPGKGRELVASISSFDDITYYPDSVEGRFTISRDNAKRMVYLQMNSLRAEDTAVYYCAAIAHQQKLSYWGQGTQVTVSS

SEQ ID NO: 432 - protein, 118 aa, Artificial Sequence
  FAP-FR3-G07 VH
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAGETAYHFNYWGQGTQVTVSS